(19) United States
(12) Patent Application Publication
(10) Pub. No.: US 2025/0120374 A1
(43) Pub. Date: Apr. 17, 2025

(54) GENETICALLY MODIFIED NON-HUMAN ANIMAL WITH HUMAN OR CHIMERIC GENES

(71) Applicant: Biocytogen Pharmaceuticals (Beijing) Co., Ltd., Beijing (CN)

(72) Inventors: Xiaofei Zhou, Beijing (CN); Jiahui Chang, Beijing (CN); Chong Li, Beijing (CN); Zhifang Bai, Beijing (CN)

(21) Appl. No.: 18/702,039

(22) PCT Filed: Oct. 25, 2022
(86) PCT No.: PCT/CN2022/127313

§ 371 Date: Apr. 17, 2024

(30) Foreign Application Priority Data
Oct. 25, 2021 (CN) 202111240593.5
Mar. 2, 2022 (CN) 202210199517.2

(51) Int. Cl. A01K 67/0276; C07K 14/54; C07K 14/715

(52) U.S. Cl. CPC A01K 67/0276; C07K 14/54; C07K 14/7155; A01K 2217/075; A01K 2227/105; A01K 2267/0325

(57) ABSTRACT

Provided are genetically modified non-human animals that express a human or chimeric (e.g., humanized) IL36A and/or IL1RL2, and methods of use thereof.

### CLAIM OF PRIORITY

This application claims the benefit of Chinese Patent Application App. No. 202111240593.5, filed on Oct. 25, 2021 and Chinese Patent Application App. No. 202210199517.2, filed on Mar. 2, 2022. The entire contents of the foregoing applications are incorporated herein by reference.

### TECHNICAL FIELD

This disclosure relates to genetically modified animals expressing human or chimeric (e.g., humanized) IL1RL2 and/or IL36A, and methods of use thereof.

### BACKGROUND

The traditional drug research and development typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results.

Therefore, the development of humanized animal models that are suitable for human antibody screening and evaluation will significantly improve the efficiency of new drug development and reduce the cost for drug research and development.

### SUMMARY

This disclosure is related to an animal model with human or chimeric IL1RL2 and/or IL36A proteins. The animal model can express human or chimeric IL1RL2 (e.g., humanized IL1RL2) protein and/or human or chimeric IL36A (e.g., humanized IL36A) protein in its body. It can be used in the studies on the function of IL1RL2 and/or IL36A genes, and can be used in the screening and evaluation of IL1RL2 and/or IL36A signaling pathway modulators (e.g., anti-human IL1RL2 antibodies or anti-human IL36A antibodies). In addition, the animal models prepared by the methods described herein can be used in drug screening, pharmacodynamics studies, treatments for immune-related diseases, and cancer therapy for human IL1RL2 and/or IL36A target sites; they can also be used to facilitate the development and design of new drugs, and save time and cost. In summary, this disclosure provides a powerful tool for studying the function of IL1RL2 and/or IL36A proteins and a platform for screening cancer drugs.

In one aspect, the disclosure is related to a genetically-modified, non-human animal whose genome comprises at least one chromosome comprising a sequence encoding a human or chimeric Interleukin-36 alpha (IL36A).

In some embodiments, the sequence encoding the human or chimeric IL36A is operably linked to an endogenous regulatory element (e.g., endogenous 5′ UTR and/or 3′ UTR) at the endogenous IL36A gene locus in the at least one chromosome.

In some embodiments, the sequence encoding a human or chimeric IL36A comprises a sequence encoding an amino acid sequence that is at least 70%, 75%, 80%, 85%, 90%, 95%, 99%, or 100% identical to human IL36A (SEQ ID NO: 30).

In some embodiments, the animal is a mammal, e.g., a monkey, a rodent, a mouse, or a rat.

In some embodiments, the animal is a mouse.

In some embodiments, the animal does not express endogenous IL36A or expresses a decreased level of endogenous IL36A as compared to IL36A expression level in a wild-type animal.

In some embodiments, the animal has one or more cells expressing human or chimeric IL36A.

In some embodiments, the animal has one or more cells expressing human or chimeric IL36A, and the expressed human or chimeric IL36A can bind to endogenous IL36A receptor (IL1RL2), inducing downstream signaling pathways.

In some embodiments, the animal has one or more cells expressing human or chimeric IL36A, and the expressed human or chimeric IL36A can bind to human or humanized IL36A receptor (IL1RL2), inducing downstream signaling pathways.

In one aspect, the disclosure is related to a genetically-modified, non-human animal, wherein the genome of the animal comprises a replacement of a sequence encoding a region of endogenous IL36A with a sequence encoding a corresponding region of human IL36A at an endogenous IL36A gene locus.

In some embodiments, the sequence encoding the corresponding region of human IL36A is operably linked to an endogenous regulatory element at the endogenous IL36A locus, and one or more cells of the animal express a human or chimeric IL36A.

In some embodiments, the animal does not express endogenous IL36A or expresses a decreased level of endogenous IL36A as compared to IL36A expression level in a wild-type animal.

In some embodiments, the replaced sequence encodes the full-length protein of IL36A.

In some embodiments, the animal is a mouse, and the replaced endogenous IL36A region comprises a portion of exon 2, exon 3, exon 4, and/or a portion of exon 5 of the endogenous mouse IL36A gene.

In some embodiments, the animal is heterozygous or homozygous with respect to the replacement at the endogenous IL36A gene locus.

In one aspect, the disclosure is related to a non-human animal comprising at least one cell comprising a nucleotide sequence encoding a human or humanized IL36A polypeptide, wherein the human or humanized IL36A polypeptide comprises at least 50, 100, 110, 120, 130, 140, 150, 155, 156, 157, or 158 contiguous amino acid residues that are identical to the corresponding contiguous amino acid sequence of a human IL36A, wherein the animal expresses the human or humanized IL36A polypeptide.

In some embodiments, the nucleotide sequence is operably linked to an endogenous IL36A regulatory element of the animal.

In some embodiments, the nucleotide sequence is integrated to an endogenous IL36A gene locus of the animal.

In some embodiments, the human or humanized IL36A polypeptide has at least one mouse IL36A activity and/or at least one human IL36A activity.

In one aspect, the disclosure is related to a method for making a genetically-modified, non-human animal, comprising: replacing in at least one cell of the animal, at an endogenous IL36A gene locus, a sequence encoding a region of an endogenous IL36A with a sequence encoding a corresponding region of human IL36A.

In some embodiments, the sequence encoding the corresponding region of human IL36A comprises a portion of exon 1, exon 2, exon 3, and a portion of exon 4 of a human IL36A gene.

In some embodiments, the sequence encoding the corresponding region of human IL36A comprises at least 50, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 474, or 477 nucleotides of exon 1, exon 2, exon 3, and/or exon 4 of a human IL36A gene.

In some embodiments, the sequence encoding the corresponding region of human IL36A encodes a sequence that is at least 80%, 85%, 90%, 95%, 96%, 97%, 98%, 99%, or 100% identical to SEQ ID NO: 30.

In some embodiments, the animal is a mouse, and the locus is a portion of exon 2, exon 3, exons 4, and/or a portion of exon 5 of the mouse IL36A gene.

In one aspect, the disclosure is related to a method of making a genetically-modified non-human animal cell that expresses a human or chimeric IL36A, the method comprising: replacing, at an endogenous mouse IL36A gene locus, a nucleotide sequence encoding a region of endogenous IL36A with a nucleotide sequence encoding a corresponding region of human IL36A, thereby generating a genetically-modified non-human animal cell that includes a nucleotide sequence that encodes the human or chimeric IL36A, wherein the animal cell expresses the human or chimeric IL36A.

In some embodiments, the animal is a mouse.

In some embodiments, the nucleotide sequence encoding the human or chimeric IL36A is operably linked to an endogenous IL36A regulatory region, e.g., a promoter.

In some embodiments, the animal further comprises a sequence encoding an additional human or chimeric protein, e.g., IL36A receptor (IL1RL2), IL33, IL7R, IL6, IL12, IL23, or Tumor necrosis factor alpha (TNF-α).

In some embodiments, the additional human or chimeric protein is IL1RL2.

In some embodiments, the animal or mouse further comprises a sequence encoding an additional human or chimeric protein, e.g., IL1RL2, IL33, IL7R, IL6, IL12, IL23, or TNF-α.

In some embodiments, the additional human or chimeric protein is IL1RL2.

In one aspect, the disclosure is related to a genetically-modified, non-human animal whose genome comprises at least one chromosome comprising a sequence encoding a human or chimeric interleukin-1 receptor-like 2 (IL1RL2).

In some embodiments, the sequence encoding the human or chimeric IL1RL2 is operably linked to an endogenous regulatory element at the endogenous IL1RL2 gene locus in the at least one chromosome.

In some embodiments, the sequence encoding a human or chimeric IL1RL2 comprises a sequence encoding an amino acid sequence that is at least 70%, 75%, 80%, 85%, 90%, 95%, 99%, or 100% identical to human IL1RL2 (SEQ ID NO: 2).

In some embodiments, the sequence encoding a human or chimeric IL1RL2 comprises a sequence encoding an amino acid sequence that is at least 70%, 75%, 80%, 85%, 90%, 95%, 99%, or 100% identical to amino acids 1-356 of human IL1RL2 (SEQ ID NO: 2).

In some embodiments, the sequence encoding a human or chimeric IL1RL2 comprises a sequence encoding an amino acid sequence that is at least 70%, 75%, 80%, 85%, 90%, 95%, 99%, or 100% identical to SEQ ID NO: 10.

In some embodiments, the animal is a mammal, e.g., a monkey, a rodent, a mouse, or a rat.

In some embodiments, the animal is a mouse.

In some embodiments, the animal does not express endogenous IL1RL2 or expresses a decreased level of endogenous IL1RL2 as compared to IL1RL2 expression level in a wild-type animal.

In some embodiments, the animal has one or more cells expressing human or chimeric IL1RL2.

In some embodiments, the animal has one or more cells expressing human or chimeric IL1RL2 that can bind to endogenous IL36A to induce downstream signaling pathways.

In some embodiments, the animal has one or more cells expressing human or chimeric IL1RL2 that can bind to human IL36A to induce downstream signaling pathways.

In one aspect, the disclosure is related to a genetically-modified, non-human animal, wherein the genome of the animal comprises an insertion of a sequence encoding a human or chimeric IL1RL2 at an endogenous IL1RL2 gene locus.

In some embodiments, the sequence encoding a human or chimeric IL1RL2 is operably linked to an endogenous regulatory element at the endogenous IL1RL2 locus, and one or more cells of the animal express the human or chimeric IL1RL2.

In some embodiments, the animal does not express endogenous IL1RL2 or expresses a decreased level of endogenous IL1RL2 as compared to IL1RL2 expression level in a wild-type animal.

In some embodiments, the sequence encoding a human or chimeric IL1RL2 is inserted within exon 1, intron 1, exon 2, intron 2, exon 3, intron 3, exon 4, intron 4, exon 5, intron 5, exon 6, intron 6, exon 7, intron 7, exon 8, intron 8, exon 9, intron 9, exon 10, intron 10, or exon 11 of endogenous IL1RL2 gene.

In some embodiments, the sequence encoding a human or chimeric IL1RL2 is inserted within exon 2 of endogenous IL1RL2 gene.

In some embodiments, the sequence encoding a human or chimeric IL1RL2 is inserted immediately after a nucleotide corresponding to position 238 of NM_133193.4.

In some embodiments, the inserted sequence comprises, optionally from 5′ end to 3′ end:
a) a sequence encoding a signal peptide;
b) a first sequence comprising a sequence encoding a humanized IL1RL2 extracellular region and a humanized IL1RL2 transmembrane region;
c) a second sequence comprising a sequence encoding all or a portion of the cytoplasmic region of an endogenous IL1RL2;
d) a regulatory sequence of endogenous IL1RL2 gene (e.g., 3′ UTR); and
e) optionally one or more auxiliary sequences (e.g., WPRE, lox2, STOP, and/or polyA).

In some embodiments, the sequence encoding a signal peptide encodes an amino acid that is at least 70%, 75%, 80%, 85%, 90%, 95%, 99%, or 100% identical to amino acids 1-19 of SEQ ID NO: 2.

In some embodiments, the first sequence further comprises a sequence encoding at least 1, at least 2, at least 3, at least 4, at least 5, or at least 6 amino acids of the transmembrane region of a humanized IL1RL2.

In some embodiments, the first sequence is at least 70%, 75%, 80%, 85%, 90%, 95%, 99%, or 100% identical to SEQ ID NO: 5, and the second sequence is at least 70%, 75%, 80%, 85%, 90%, 95%, 99%, or 100% identical to SEQ ID NO: 6.

In some embodiments, the first sequence encodes an amino acid that is at least 70%, 75%, 80%, 85%, 90%, 95%, 99%, or 100% identical to amino acids 1-356 of SEQ ID NO: 2, and the second sequence encodes an amino acid sequence that is at least 70%, 75%, 80%, 85%, 90%, 95%, 99%, or 100% identical to amino acids 360-574 of SEQ ID NO: 1.

In some embodiments, the one or more auxiliary sequences comprise a PolyA sequence.

In some embodiments, the animal is heterozygous or homozygous with respect to the insertion at the endogenous IL1RL2 gene locus.

In one aspect, the disclosure is related to a non-human animal comprising at least one cell comprising a nucleotide sequence encoding a humanized IL1RL2 polypeptide, wherein the humanized IL1RL2 polypeptide comprises at least 50 contiguous amino acid residues that are identical to the corresponding contiguous amino acid sequence of a human IL1RL2, wherein the animal expresses the humanized IL1RL2 polypeptide.

In some embodiments, the humanized IL1RL2 polypeptide has at least 50, 100, 150, 160, 170, 180, 190, 200, 210, 220, 230, 240, 250, 260, 270, 280, 290, 300, 310, 320, 330, 340, 350, 351, 352, 353, 354, 355, or 356 contiguous amino acid residues that are identical to the corresponding contiguous amino acid sequence of human IL1RL2 extracellular and transmembrane regions.

In some embodiments, the humanized IL1RL2 polypeptide comprises a sequence that is at least 90%, 95%, or 99% identical to amino acids 1-356 of SEQ ID NO: 2.

In some embodiments, the nucleotide sequence is operably linked to an endogenous IL1RL2 regulatory element of the animal.

In some embodiments, the nucleotide sequence is integrated to an endogenous IL1RL2 gene locus of the animal.

In some embodiments, the humanized IL1RL2 polypeptide has at least one mouse IL1RL2 activity and/or at least one human IL1RL2 activity.

In one aspect, the disclosure is related to a method of making a genetically-modified non-human animal cell that expresses a chimeric IL1RL2, the method comprising: inserting at an endogenous IL1RL2 gene locus (e.g., exon 2 of endogenous IL1RL2 gene), a nucleotide sequence comprising, optionally from 5′ end to 3′ end:
a) a first sequence comprising a sequence encoding all or a portion of the extracellular region and transmembrane region of a human IL1RL2;
b) a second sequence comprising a sequence encoding all or a portion of the cytoplasmic region of an endogenous IL1RL2;
c) a regulatory sequence of endogenous IL1RL2 gene (e.g., 3′ UTR); and
d) optionally one or more auxiliary sequences (e.g., WPRE, lox2, STOP, and/or polyA);
thereby generating a genetically-modified non-human animal cell that includes a nucleotide sequence that encodes the chimeric IL1RL2, wherein the animal cell expresses the chimeric IL1RL2.

In some embodiments, the animal is a mouse.

In some embodiments, the nucleotide sequence encoding the chimeric IL1RL2 polypeptide is operably linked to an endogenous IL1RL2 regulatory region, e.g., promoter.

In some embodiments, the animal further comprises a sequence encoding an additional human or chimeric protein, e.g., IL36A, IL33, IL7R, IL6, IL12, IL23, or Tumor necrosis factor alpha (TNF-α).

In some embodiments, the additional human or chimeric protein is IL36A.

In some embodiments, the animal or mouse further comprises a sequence encoding an additional human or chimeric protein, e.g., IL36A, IL33, IL7R, IL6, IL12, IL23, or TNF-α.

In some embodiments, the additional human or chimeric protein is IL36A.

In one aspect, the disclosure is related to a method of determining effectiveness of a therapeutic agent for treating an immune disorder, comprising: administering the therapeutic agent to the animal described herein, wherein the animal has the immune disorder; and determining effects of the therapeutic agent in treating the immune disorder.

In some embodiments, the immune disorder is psoriasis.

In some embodiments, the psoriasis is induced by treating the animal with imiquimod (IMQ).

In some embodiments, the effects are evaluated by comparing skin damage parameters (e.g., rash and sesquamation) of the animal with an animal that is not treated with the therapeutic agent.

In some embodiments, the therapeutic agent is an anti-IL36A antibody, an anti-IL1RL2 antibody, and/or a corticosteroid (e.g., dexamethasone).

In one aspect, the disclosure is related to a method of determining effectiveness of a therapeutic agent for reducing an inflammation, comprising: administering the therapeutic agent to the animal described herein, wherein the animal has the inflammation; and determining effects of the therapeutic agent for reducing the inflammation.

In one aspect, the disclosure is related to a method of determining effectiveness of a therapeutic agent for treating an autoimmune disease, comprising: administering the therapeutic agent to the animal described herein, wherein the animal has the autoimmune disease; and determining effects of the therapeutic agent for treating the autoimmune disease.

In some embodiments, the autoimmune disease is inflammatory arthritis, eczema, eosinophilic esophagitis, rheumatoid arthritis, Crohn's disease, systemic lupus erythematosus, ankylosing spondylitis, inflammatory bowel diseases (IBD), ulcerative colitis, multiple sclerosis, systemic juvenile idiopathic arthritis (SJIA), and/or scleroderma.

In some embodiments, the therapeutic agent is an anti-IL36A antibody, an anti-IL1RL2 antibody, or a corticosteroid (e.g., dexamethasone).

In one aspect, the disclosure is related to a method of determining toxicity of a therapeutic agent comprising: a) administering the therapeutic agent to the animal described herein; and b) determining effects of the therapeutic agent to the animal.

In some embodiments, the therapeutic agent is an anti-IL36A antibody or an anti-IL1RL2 antibody.

In some embodiments, determining effects of the therapeutic agent to the animal involves measuring the body weight, red blood cell count, hematocrit, and/or hemoglobin of the animal.

In one aspect, the disclosure is related to a protein comprising an amino acid sequence, wherein the amino acid sequence is one of the following:
an amino acid sequence set forth in SEQ ID NO: 1, 2, 10, 29, or 30;
an amino acid sequence that is at least 90% identical to SEQ ID NO: 1, 2, 10, 29, or 30;
an amino acid sequence that is at least 91%, 92%, 93%, 94%, 95%, 96%, 97%, 98%, or 99% identical to SEQ ID NO: 1, 2, 10, 29, or 30;
an amino acid sequence that is different from the amino acid sequence set forth in SEQ ID NO: 1, 2, 10, 29, or 30 by no more than 10, 9, 8, 7, 6, 5, 4, 3, 2 or 1 amino acid; and
an amino acid sequence that comprises a substitution, a deletion and/or insertion of one, two, three, four, five or more amino acids to the amino acid sequence set forth in SEQ ID NO: 1, 2, 10, 29, or 30.

In one aspect, the disclosure is related to a nucleic acid comprising a nucleotide sequence, wherein the nucleotide sequence is one of the following:
a sequence that encodes the protein described herein;
SEQ ID NO: 3, 4, 5, 6, 7, 8, 9, 11, 12, 31, 32, 33, 34, 35, 36, 37, 38, or 39;
a sequence that is at least 90% identical to SEQ ID NO: 3, 4, 5, 6, 7, 8, 9, 11, 12, 31, 32, 33, 34, 35, 36, 37, 38, or 39; and
a sequence that is at least 91%, 92%, 93%, 94%, 95%, 96%, 97%, 98%, or 99% identical to SEQ ID NO: 3, 4, 5, 6, 7, 8, 9, 11, 12, 31, 32, 33, 34, 35, 36, 37, 38, or 39.

In one aspect, the disclosure is related to a cell comprising the protein described herein and/or the nucleic acid described herein.

In one aspect, the disclosure is related to an animal comprising the protein described herein and/or the nucleic acid described herein.

In one aspect, the disclosure is related to a method for making a genetically-modified, non-human animal, comprising:
(1) providing a plasmid comprising a human or chimeric IL1RL2 gene fragment, flanked by a 5′ homology arm and a 3′ homology arm, wherein the 5′ and 3′ homology arms target an endogenous IL1RL2 gene; and
(2) modifying the genome of a fertilized egg or an embryonic stem cell by inserting the human or chimeric IL1RL2 gene fragment into the genome.

In some embodiments, the 5′ homology arm comprises SEQ ID NO: 3 and the 3′ homology arm comprises SEQ ID NO: 4.

In one aspect, the disclosure is related to a method for making a genetically-modified, non-human animal, comprising:
(1) providing a plasmid comprising a human or chimeric IL36A gene fragment, flanked by a 5′ homology arm and a 3′ homology arm, wherein the 5′ and 3′ homology arms target an endogenous IL36A gene; and
(2) modifying the genome of a fertilized egg or an embryonic stem cell by inserting the human or chimeric IL36A gene fragment into the genome.

In some embodiments, the 5′ homology arm comprises SEQ ID NO: 31 and the 3′ homology arm comprises SEQ ID NO: 32.

In one aspect, the disclosure is related to a genetically modified animal or a progeny thereof, wherein the genetically modified animal is made by a method comprising the steps of: modifying genome of an embryo of the animal by CRISPR with sgRNAs that target any one of SEQ ID NOs: 40-44 and any one of SEQ ID NOs: 45-49 to introduce a fragment of human IL36A gene, wherein an endogenous IL36A gene locus in the genome of the embryo is modified; and transplanting the embryo to a recipient mouse to produce the genetically-modified mouse, wherein the genetically-modified mouse expresses a humanized IL36A.

In some embodiments, the sgRNAs target SEQ ID NO: 41 and SEQ ID NO: 47.

In one aspect, the disclosure is related to a method for making a genetically-modified, non-human animal, comprising:
(1) providing a plasmid comprising a human IL36A gene fragment, flanked by a 5′ homology arm and a 3′ homology arm, wherein the 5′ and 3′ homology arms target an endogenous IL36A gene;
(2) providing one or more small guide RNAs (sgRNAs) that target the endogenous IL36A gene;
(3) modifying genome of a fertilized egg or an embryonic stem cell by using the plasmid of step (1), the sgRNAs of step (2), and Cas9;
(4) transplanting the fertilized egg obtained in step (3) into the oviduct of a pseudopregnant female mouse or transplanting the embryonic stem cell obtained in step (3) into a blastocyst which is then transplanted into the oviduct of a pseudopregnant female mouse to produce a child mouse that functionally expresses a humanized IL36A protein.

In some embodiments, the 5′ homology arm comprises SEQ ID NO: 38 and the 3′ homology arm comprises SEQ ID NO: 39.

In one aspect, the disclosure is related to a method for making a genetically-modified, non-human animal, comprising:
(1) providing a plasmid comprising a human or chimeric IL36A gene fragment, flanked by a 5′ homology arm and a 3′ homology arm, wherein the 5′ and 3′ homology arms target an endogenous IL36A gene;
(2) providing two small guide RNAs (sgRNAs) that target the endogenous IL36A gene; and
(3) modifying the genome of a fertilized egg or an embryonic stem cell by inserting the human or chimeric IL36A gene fragment into the genome.

In some embodiments, the first sgRNA targets SEQ ID NO: 41 and the second sgRNA targets SEQ ID NO: 47.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing mouse and human IL1RL2 gene loci (not to scale).

FIG. 2 is a schematic diagram showing humanized IL1RL2 gene locus (not to scale).

FIG. 3 is a schematic diagram showing a IL1RL2 gene targeting strategy and design of targeting vector V1 (not to scale).

FIG. 4 shows Southern Blot results of cells after recombination using the 5′ Probe, 3′ Probe, and Neo Probe. WT is a wild-type control.

FIG. 5 is a schematic diagram showing FRT recombination process in IL1RL2 gene humanized mice (not to scale).

FIGS. 6A-6D show the identification results of IL1RL2 humanized F1 generation mice. M is Marker. WT is wild type control. H₂O is water control. PC is positive control. F1-01, F1-02, F1-03 are mouse numbers.

FIGS. 7A-7C show the RT-PCR detection results. +/+means C57BL/6 wild-type mice. H/H means IL1RL2 humanized homozygous mice. H₂O is water control.

FIG. 8 shows the IHC detection results of each tissue in mice.

FIG. 9 is a schematic diagram showing mouse and human IL36A gene loci (not to scale).

FIG. 10 is a schematic diagram showing humanized IL36A gene locus (not to scale).

FIG. 11 is a schematic diagram showing a IL36A gene targeting strategy and design of targeting vector V2 (not to scale).

FIG. 12 is a schematic diagram showing a IL36A gene targeting strategy and design of targeting vector V3 (not to scale).

FIGS. 13A-13B show sgRNA activity detection results. Con is a negative control and PC is a positive control.

FIGS. 14A-14B show the identification results of IL36A humanized F0 generation mice. M is Marker. WT is wild type control. H₂O is water control. PC is positive control. F0-01 and F0-02 are mouse numbers.

FIG. 15 shows the southern blot detection results of IL36A humanized F1 generation mice. WT is the wild-type control. F1-01, F1-02, F1-03, F1-04, F1-05, F1-06, F1-07, F1-08 are the mouse numbers.

FIGS. 16A-16C show the RT-PCR detection results of IL36A gene humanized homozygous mice. +/+means C57BL/6 wild-type mice. H/H means IL1RL2 gene humanized homozygous mice. H₂O is water control.

FIG. 17 shows the western Blot detection results. +/+means C57BL/6 wild-type mice. H/H means IL36A gene humanized homozygous mice. GAPDH is glyceraldehyde-3-phosphate dehydrogenase control.

FIGS. 18A-18B show percentages of CD45+ cell subtype (A) and T cell subtype (B) in the spleen of IL1RL2 humanized homozygous mice.

FIGS. 19A-19B show percentages of CD45+ cell subtypes (A) and T cell subtypes (B) in lymph nodes of IL1RL2 humanized homozygous mice.

FIGS. 20A-20B show percentages of CD45+ cell subtype (A) and T cell subtype (B) in the blood of IL1RL2 humanized homozygous mice.

FIG. 21 shows the body weights of mice in the control group (G1), model group (G2) and administration group (G3-G5) in the in vivo psoriasis efficacy study using IL1RL2 humanized homozygous mice.

FIG. 22 shows psoriasis-like rash scores of mice in control group (G1), model group (G2) and administration group (G3-G5) in the in vivo psoriasis efficacy study using IL1RL2 humanized homozygous mice.

FIG. 23 shows psoriasis-like desquamation scores of mice in control group (G1), model group (G2) and administration group (G3-G5) in the in vivo psoriasis efficacy study using IL1RL2 humanized homozygous mice.

FIG. 24 shows the PASI composite scores of mice in control group (G1), modeling group (G2) and anti-human IL1RL2 antibody administration groups (G3-G5) in the in vivo psoriasis efficacy study using IL1RL2 humanized homozygous mice.

FIG. 25 shows the alignment between human IL1RL2 amino acid sequence (NP_003845.2; SEQ ID NO: 2) and mouse IL1RL2 amino acid sequence (NP_573456.1; SEQ ID NO: 1).

FIG. 26 shows the alignment between human IL1RL2 amino acid sequence (NP_003845.2; SEQ ID NO: 2) and rat IL1RL2 amino acid sequence (NP_598259.2; SEQ ID NO: 80).

FIG. 27 shows the alignment between human IL36A amino acid sequence (NP_055255.1; SEQ ID NO: 30) and mouse IL36A amino acid sequence (NP_062323.1; SEQ ID NO: 29).

FIG. 28 shows the alignment between human IL36A amino acid sequence (NP_055255.1; SEQ ID NO: 30) and rat IL36A amino acid sequence (NP_001100024.1; SEQ ID NO: 81.

### DETAILED DESCRIPTION

This disclosure relates to transgenic non-human animal with human or chimeric (e.g., humanized) IL1RL2 and/or IL36A, and methods of use thereof.

### IL1RL2

The human and rat IL-36R gene, also known as IL-1R6, interleukin receptor related protein 2 (IL-1Rrp2) or interleukin 1 receptor like 2 (IL-1RL2), was the sixth IL-1 receptor identified. The gene is located on chromosome 2 and its sequence is conserved in monkey, dog, cow, mouse, rat, and zebrafish. Biochemical analysis has revealed that the IL-1R family members share a similar molecular structure that consists of three structural domains with distinct functions, which help these receptors facilitate their biological activity. The common domains shared by this group of receptors include an extracellular domain, a transmembrane region, and a Toll-IL-1 receptor domain (TIR). The extracellular region contains three Immunoglobulin (Ig) domains (D1, D2, and D3). This stretch of amino acid sequence is important for cytokine recognition. The TIR domain, which resides in the cytoplasm, is essential for co-receptor binding and signal transduction. This domain has a sequence identity similar to that found in toll-like receptors (TLRs), which are also important mediators of inflammation.

Biochemical studies have revealed that IL-36R functions as a heterodimer that associates with a co-receptor called interleukin-1 receptor 3 (IL-1R3), also known as interleukin 1 receptor accessory protein (IL-1RAcP). It has also been shown that IL-36R stimulates a signal transduction event through a mechanism that involves the actions of an adaptor protein myeloid differentiation primary response gene 88 (MyD88) and the interleukin-1 receptor associated kinase (IRAK). In addition, several known agonists (IL-36α, IL-36β, and IL-36γ) and antagonists (IL-36Ra, IL-38) have been shown to modulate its activity. Signal transduction through this pathway leads to mitogen-activated protein kinase (MAPK) induced Activated Protein-1 (AP) and nuclear factor kappa-light-chain-enhancer of activated B cells (NFKB) dependent upregulation of pro-inflammatory gene expression.

IL-36R is highly expressed in the skin and epithelial cells, which are the major cell types involved in skin psoriasis. According to the RNA-seq alignment reported by BioProject: (PRJEB4337), the human isoform of IL-36R is highly expressed in the esophagus, thyroid, kidney, skin, adrenal gland, and the gall bladder.

IL-36R signaling has been shown to influence the cellular inflammatory response in multiple ways. The multifaceted mechanism attributed to IL-36R includes inducing cytokine production in cells such as skin keratinocytes, endothelial cells, and lung fibroblasts. It also produces pro-inflammatory responses in immune cells. For example, microarray analysis showed that IL-36β treatment stimulated an induction of cytokines and chemokines (namely, granulocyte colony-stimulating factor (G-CSF), interleukin 17C (IL-17C), C-C motif chemokine ligand 20 (CCL20), and interleukin 8 (IL-8)) in normal primary keratinocytes isolated from humans. Similar pro-inflammatory effects were observed in endothelial cells, which exhibited elevated CCL20, C-C motif chemokine ligand 2 (CCL2), IL-8, and vascular cell adhesion molecule 1 (VCAM 1) levels when stimulated with IL-36γ. Likewise, in healthy lung fibroblasts, upregulation of pro-inflammatory markers such IL-8 and CCL20 was potentiated through the activation of the IL-36R pathway via IL-36γ. IL-36R associated pro-inflammatory effects has also been demonstrated in cell types such as dendritic cells, macrophages, human leukemic monocytes (THP-1 cells), neurons, and glial cells. In addition to aiding in the production of cytokines, IL-36R signaling also contributes to inflammation by modulating lymphocyte activity. This membrane receptor is expressed on a variety of leukocytes that have the capacity to respond to IL-36 cytokine stimulation. For example, IL-36 treatment can provoke an immune response in dendritic and T cells, as well as stimulate their differentiation.

A detailed description of IL1RL2 and its function can be found, e.g., in Melton, Elaina, and Hongyu Qiu. “Interleukin-36 cytokine/receptor signaling: a new target for tissue fibrosis.” International Journal of Molecular Sciences 21.18 (2020): 6458; Furue, Kazuhisa, et al. “Highlighting interleukin-36 signalling in plaque psoriasis and pustular psoriasis.” Acta dermato-venereologica 98.1 (2018): 5-13, each of which is incorporated by reference in its entirety.

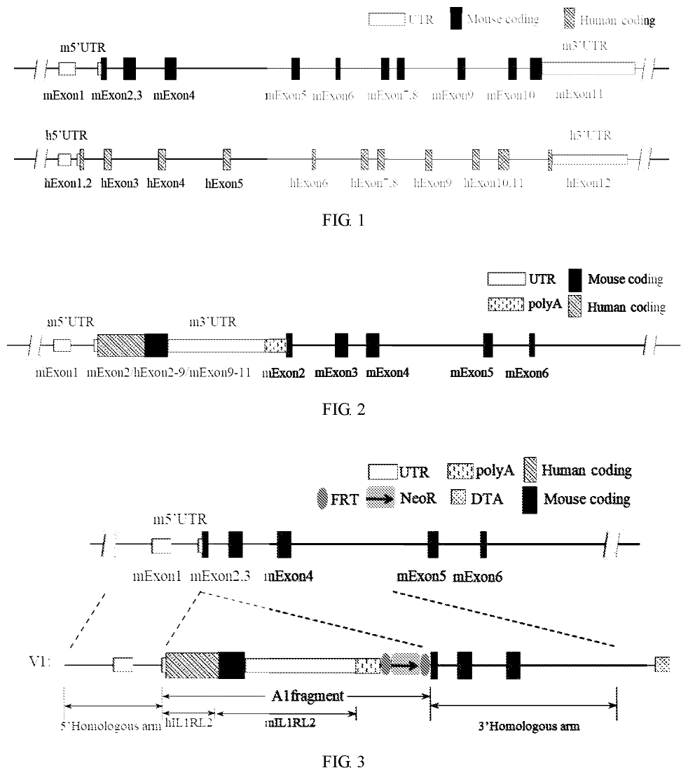

In human genomes, IL1RL2 gene (gene ID: 8808) locus has 12 exons, exon 1, exon 2, exon 3, exon 4, exon 5, exon 6, exon 7, exon 8, exon 9, exon 10, exon 11 and exon 12 (FIG. 1). The IL1RL2 protein also has a signal peptide. The nucleotide sequence for human IL1RL2 mRNA is NM_003854.4, and the amino acid sequence for human IL1RL2 is NP_003845.2 (SEQ ID NO: 2). The location for each exon and each region in human IL1RL2 nucleotide sequence and amino acid sequence is listed below:

TABLE 1

| | | NP_003845.2 |
|---|---|---|
| Human IL1RL2 | NM_003854.4 | 575aa (SEQ |
| (approximate location) | 2565 bp | ID NO: 2) |
| Exon 1 | 1-64 | — |
| Exon 2 | 65-134 | 1-19 |
| Exon 3 | 135-369 | 20-98 |
| Exon 4 | 370-565 | 99-163 |
| Exon 5 | 566-725 | 164-216 |
| Exon 6 | 726-800 | 217-241 |
| Exon 7 | 801-930 | 242-285 |
| Exon 8 | 931-1067 | 286-330 |
| Exon 9 | 1068-1211 | 331-378 |
| Exon 10 | 1212-1373 | 379-432 |
| Exon 11 | 1374-1754 | 433-559 |
| Exon 12 | 1755-2565 | 560-575 |
| Signal peptide | 77-133 | 1-19 |
| Extracellular region | 134-1081 | 20-335 |
| Transmembrane region | 1082-1144 | 336-356 |
| Cytoplasmic region | 1145-1801 | 357-575 |
| Donor region in Example | 77-1144 | 1-356 |

The human IL1RL2 gene (Gene ID: 8808) is located in Chromosome 2, which is located from 102186973 to 102242910 of NC_000002.12 (GRCh38.p13 (GCF_000001405.39)). The 5′-UTR is from 102,187,023 to 102,187,086 and 102,187,856 to 102,187,867, exon 1 is from 102,187,023 to 102,187,086, the first intron is from 102,187,087 to 102,187,855, exon 2 is from 102,187,856 to 102,187,925, the second intron is from 102,187,926 to 102,189,075, exon 3 is from 102,189,076 to 102,189,310, the third intron is from 102,189,311 to 102,191,924, exon 4 is from 102,191,925 to 102,192,120, the fourth intron is from 102,192,121 to 102,201,555, exon 5 is from 102,201,556 to 102,201,715, the fifth intron is from 102,201,716 to 102,212,099, exon 6 is from 102,212,100 to 102,212,174, the sixth intron is from 102,212,175 to 102,218,952, exon 7 is from 102,218,953 to 102,219,082, the seventh intron is from 102,219,083 to 102,219,880, exon 8 is from 102,219,881 to 102,220,017, the eighth intron is from 102,220,018 to 102,225,897, exon 9 is from 02,225,898 to 1,102,226,041, the ninth intron is from 102,226,042 to 102,232,962, exon 10 is from 102,232,963 to 102,233,124, the tenth intron is from 102,233,125 to 102,234,896, exon 11 is from 102,234,897 to 102,235,277, the 11thintron is from 102,235,278 to 102,239,191, exon 12 is from 102,239,192 to 102,240,002, and the 3′-UTR is from 102,240,241 to 102,240,002, based on transcriptNM_003854.4. All relevant information for human IL1RL2 locus can be found in the NCBI website with Gene ID: 8808, which is incorporated by reference herein in its entirety.

In mice, IL1RL2 gene locus has 11 exons, exon 1, exon 2, exon 3, exon 4, exon 5, exon 6, exon 7, exon 8, exon 9, exon 10 and exon 11 (FIG. 1). The mouse IL1RL2 protein also has a signal peptide. The nucleotide sequence for mouse IL1RL2 mRNA is NM_133193.4, the amino acid sequence for mouse IL1RL2 is NP_573456.1 (SEQ ID NO: 1). The location for each exon and each region in the mouse IL1RL2 nucleotide sequence and amino acid sequence is listed below:

TABLE 2

| Mouse IL1RL2 | NM_133193.4 | NP_573456.1 |
|---|---|---|
| (approximate location) | 4072 bp | 574aa (SEQ ID NO: 1) |
| Exon 1 | 1-231 | — |
| Exon 2 | 232-301 | 1-21 |
| Exon 3 | 302-536 | 22-100 |
| Exon 4 | 537-735 | 101-166 |
| Exon 5 | 736-898 | 167-220 |
| Exon 6 | 899-970 | 221-244 |
| Exon 7 | 971-1100 | 245-288 |
| Exon 8 | 1101-1237 | 289-333 |
| Exon 9 | 1238-1381 | 334-381 |
| Exon 10 | 1382-1543 | 382-435 |
| Exon 11 | 1544-4072 | 436-574 |
| Signal peptide | 238-300 | 1-21 |
| Extracellular region | 301-1251 | 22-338 |
| Transmembrane region | 1252-1314 | 339-359 |
| Cytoplasmic region | 1315-1959 | 360-574 |
| Replaced region in Example | 238-240 | 1 |

The mouse IL1RL2 gene (Gene ID: 107527) is located in Chromosome 1 of the mouse genome, which is located from 40324589 to 40367555 of NC_000067.6 (GRCm38.p6 (GCF_000001635.26)). The 5′-UTR is from 40,324,627 to U.S. Pat. Nos. 40,324,819 and 40,324,820 to 40,324,825, exon 1 is from 40,324,627 to 40,324,819, the first intron is from 40,324,820 to 40,326,488, exon 2 is from 40,326,489 to 40,326,558, the second intron is from 40,326,559 to 40,327,371, exon 3 is from 40,327,372 to 40,327,606, the third intron is from 40,327,607 to 40,328,970, exon 4 is from 40,328,971 to 40,329,169, the fourth intron is from 40,329,170 to 40,343,027, exon 5 is from 40,343,028 to 40,343,190, the fifth intron is from 40,343,191 to 40,345,805, exon 6 is from 40,345,806 to 40,345,877, the sixth intron is from 40,345,878 to 40,350,935, exon 7 is from 40,350,936 to 40,351,065, the seventh intron is from 40,351,066 to 40,351,761, exon 8 is from 40,351,762 to 40,351,898, the eighth intron is from 40,351,899 to 40,356,818, exon 9 is from 40,356,819 to 40,356,962, the ninth intron is from 40,356,963 to 40,363,191, exon 10 is from 40,363,192 to 40,363,353, the tenth intron is from 40,363,354 to 40,365,026, exon 11 is from 40,365,027 to 40,365,471, and the 3′-UTR is from 40,365,446 to 40,365,471, based on transcript NM_133193.4. All relevant information for mouse IL1RL2 locus can be found in the NCBI website with Gene ID: 107527, which is incorporated by reference herein in its entirety.

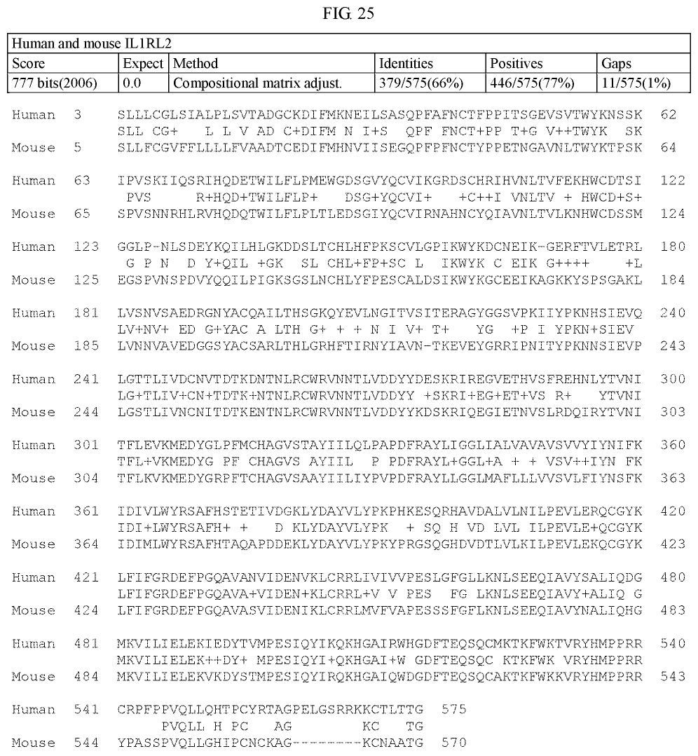

FIG. 25 shows the alignment between human IL1RL2 amino acid sequence (NP_003845.2; SEQ ID NO: 2) and mouse IL1RL2 amino acid sequence (NP_573456.1; SEQ ID NO: 1). Thus, the corresponding amino acid residue or region between human and mouse IL1RL2 can be found in FIG. 25.

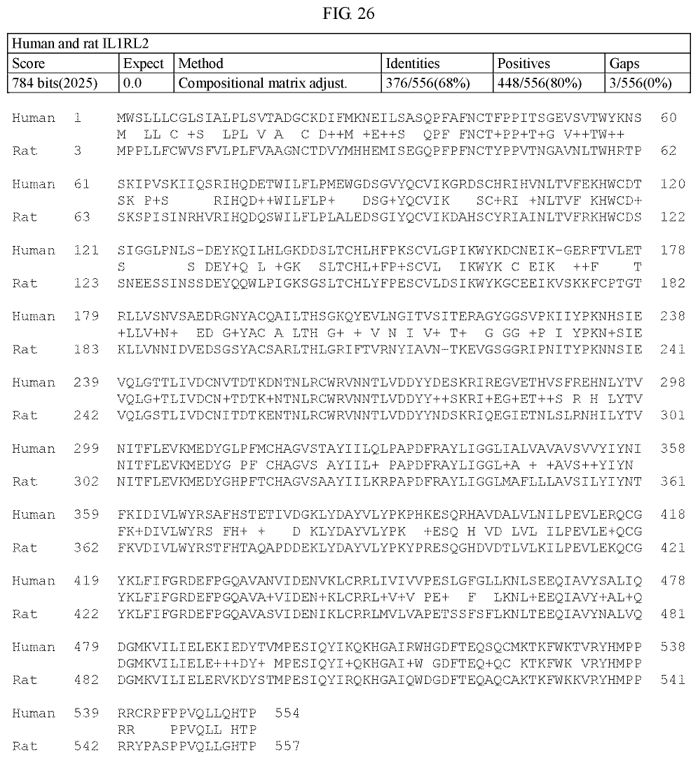

IL1RL2 genes, proteins, and locus of the other species are also known in the art. For example, the gene ID for IL1RL2 in Rattus norvegicus (rat) is 171106, the gene ID for IL1RL2 in Macaca mulatta (Rhesus monkey) is 711812, the gene ID for IL1RL2 in Canis lupus familiaris (dog) is 611453, and the gene ID for IL1RL2 in Sus scrofa (pig) is 106509756. The relevant information for these genes (e.g., intron sequences, exon sequences, amino acid residues of these proteins) can be found, e.g., in NCBI database, which is incorporated by reference herein in its entirety. FIG. 26 shows the alignment between human IL1RL2 amino acid sequence (NP_003845.2; SEQ ID NO: 2) and rat IL1RL2 amino acid sequence (NP_598259.2; SEQ ID NO: 80). Thus, the corresponding amino acid residue or region between human and rodent IL1RL2 can be found in FIG. 26.

The present disclosure provides human or chimeric (e.g., humanized) IL1RL2 nucleotide sequence and/or amino acid sequences. In some embodiments, the entire sequence of mouse exon 1, intron 1, exon 2, intron 2, exon 3, intron 3, exon 4, intron 4, exon 5, intron 5, exon 6, intron 6, exon 7, intron 7, exon 8, intron 8, exon 9, intron 9, exon 10, intron 10, exon 11, signal peptide, extracellular region, transmembrane region, and/or cytoplasmic region are replaced by human sequences. In some embodiments, a “region” or “portion” of mouse exon 1, intron 1, exon 2, intron 2, exon 3, intron 3, exon 4, intron 4, exon 5, intron 5, exon 6, intron 6, exon 7, intron 7, exon 8, exon 9, exon 10, exon 11, signal peptide, extracellular region, transmembrane regions, and/or cytoplasmic regions are replaced by human sequences. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1100, 1200, 1300, 1330, 1350, 1400, 1500, 1600, 1700, 1800, 1900, 2000, 2200, 2400, 2600, 2800, 3000, 3200, 3400, 3600, 3800, 4000, or 4072 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 310, 320, 330, 340, 350, 360, 370, 380, 390, 400, 410, 420, 430, 440, 450, 460, 470, 480, 490, 500, 510, 520, 530, 540, 550, 560, 570 or 574 amino acid residues. In some embodiments, the “region” or “portion” can be at least 50%, 55%, 60%, 65%, 70%, 75%, 80%, 85%, 90%, 95%, or 99% identical to exon 1, intron 1, exon 2, intron 2, exon 3, intron 3, exon 4, intron 4, exon 5, intron 5, exon 6, intron 6, exon 7, intron 7, exon 8, intron 8, exon 9, intron 9, exon 10, intron 10, exon 11, signal peptide, extracellular region, transmembrane region, or cytoplasmic region. In some embodiments, a region, a portion, or the entire sequence of mouse exon 1, intron 1, exon 2, intron 2, exon 3, intron 3, exon 4, intron 4, exon 5, intron 5, exon 6, intron 6, exon 7, intron 7, exon 8, intron 8, exon 9, intron 9, exon 10, intron 10, and/or exon 11 is replaced by a sequence including a region, a portion, or the entire sequence of the human exon 1, intron 1, exon 2, intron 2, exon 3, intron 3, exon 4, intron 4, exon 5, intron 5, exon 6, intron 6, exon 7, intron 7, exon 8, intron 8, exon 9, intron 9, exon 10, intron 10, exon 11, intron 11, and/or exon 12. In some embodiments, the extracellular region described herein includes the signal peptide. In some embodiments, the extracellular region described herein does not include the signal peptide.

In some embodiments, a “region” or “portion” of the signal peptide, extracellular region, transmembrane region, cytoplasmic region, exon 1, intron 1, exon 2, intron 2, exon 3, intron 3, exon 4, intron 4, exon 5, intron 5, exon 6, intron 6, exon 7, intron 7, exon 8, intron 8, exon 9, intron 9, exon 10, intron 10 and/or exon 11 of endogenous IL1RL2 protein or endogenous IL1RL2 gene is deleted.

In some embodiments, the present disclosure is related to a genetically-modified, non-human animal whose genome comprises a chimeric (e.g., humanized) IL1RL2 nucleotide sequence. In some embodiments, the chimeric (e.g., humanized) IL1RL2 nucleotide sequence encodes a IL1RL2 protein comprising an human or humanized IL1RL2 signal peptide, a human or humanized IL1RL2 extracellular region, a human or humanized IL1RL2 transmembrane regions, and an endogenous IL1RL2 cytoplasmic region. In some embodiments, the chimeric (e.g., humanized) IL1RL2 nucleotide sequence encodes a IL1RL2 protein comprising an human or humanized D1, an human or humanized D2, and/or an human or humanized D3.

In some embodiments, the encoded protein comprises a sequence that is at least 80%, 85%, 90%, 95%, or 100% identical to SEQ ID NO: 1, 2, or 10. In some embodiments, the genome of the animal comprises a sequence that is at least 80%, 85%, 90%, 95%, or 100% identical to SEQ ID NOs: 3, 4, 5, 6, 7, 8, 11, and/or 12.

In some embodiments, the genetically-modified non-human animal described herein comprises a sequence encoding a human or humanized IL1RL2 protein. In some embodiments, the IL1RL2 protein comprises a signal peptide, an extracellular region, a transmembrane region, and a cytoplasmic region. In some embodiments, the humanized IL1RL2 protein comprises a human or humanized IL1RL2 signal peptide. For example, the human or humanized IL1RL2 signal peptide comprises a sequence that is at least 70%, 80%, 85%, 90%, 95%, or 100% identical to amino acids 1-19 of SEQ ID NO: 2. In some embodiments, the humanized IL1RL2 protein comprises an endogenous IL1RL2 signal peptide. For example, the endogenous IL1RL2 signal peptide comprises a sequence that is at least 70%, 80%, 85%, 90%, 95%, or 100% identical to amino acids 1-21 of SEQ ID NO: 1. In some embodiments, the humanized IL1RL2 protein comprises a human or humanized IL1RL2 extracellular region. For example, the human or humanized IL1RL2 extracellular region comprises a sequence that is at least 70%, 80%, 85%, 90%, 95%, or 100% identical to amino acids 20-335 of SEQ ID NO: 2. In some embodiments, the humanized IL1RL2 protein comprises an endogenous IL1RL2 extracellular region. For example, the endogenous IL1RL2 extracellular region comprises a sequence that is at least 70%, 80%, 85%, 90%, 95%, or 100% identical to amino acids 22-338 of SEQ ID NO: 1. In some embodiments, the humanized IL1RL2 protein comprises a human or humanized IL1RL2 transmembrane region. For example, the human or humanized IL1RL2 transmembrane region comprises a sequence that is at least 70%, 80%, 85%, 90%, 95%, or 100% identical to amino acids 336-356 of SEQ ID NO: 2. In some embodiments, the humanized IL1RL2 protein comprises an endogenous IL1RL2 transmembrane region. For example, the endogenous IL1RL2 transmembrane region comprises a sequence that is at least 70%, 80%, 85%, 90%, 95%, or 100% identical to amino acids 339-359 of SEQ ID NO: 1. In some embodiments, the humanized IL1RL2 protein comprises a human or humanized IL1RL2 cytoplasmic region. For example, the human or humanized IL1RL2 cytoplasmic region comprises a sequence that is at least 70%, 80%, 85%, 90%, 95%, or 100% identical to amino acids 357-575 of SEQ ID NO: 2. In some embodiments, the humanized IL1RL2 protein comprises an endogenous IL1RL2 cytoplasmic region. For example, the endogenous IL1RL2 cytoplasmic region comprises a sequence that is at least 70%, 80%, 85%, 90%, 95%, or 100% identical to amino acids 360-574 of SEQ ID NO: 1.

In some embodiments, the genetically-modified non-human animal described herein comprises a human or humanized IL1RL2 gene. In some embodiments, the humanized IL1RL2 gene locus comprises from 5′ end to 3′ end: endogenous exons 1, 2, a portion of endogenous exon 3, a portion (e.g., nucleotides 77-134) of human exon 2, human exons 2-8, a portion (e.g., nucleotides 1068-1144) of human exon 9, a portion of endogenous exon 9, endogenous exons 10-11, a portion of endogenous exon 3, and/or endogenous exons 4-11. In some embodiments, the genetically-modified non-human animal described herein comprises a human or humanized IL1RL2 gene. In some embodiments, the humanized IL1RL2 gene further includes a polyA sequence. In some embodiments, the humanized IL1RL2 gene comprises human or humanized 5′ UTR. In some embodiments, the humanized IL1RL2 gene comprises human or humanized 3′ UTR. In some embodiments, the humanized IL1RL2 gene comprises endogenous 5′ UTR. In some embodiments, the humanized IL1RL2 gene comprises endogenous 3′ UTR.

In some embodiments, the genetically-modified non-human animal described herein comprises an insertion in its genome, at an endogenous IL1RL2 gene locus, of a sequence encoding a human or humanized IL1RL2 protein. In some embodiments, the inserted sequence comprises one or more sequences selected from: all or a portion of exon 1, exon 2, exon 3, exon 4, exon 5, exon 6, exon 7, exon 8, exon 9, exon 10, exon 11, and/or exon 12 of human IL1RL2 gene; and/or all or a portion of exon 1, exon 2, exon 3, exon 4, exon 5, exon 6, exon 7, exon 8, exon 9, exon 10, and/or exon 11 of endogenous IL1RL2 gene (e.g., mouse IL1RL2 gene). In some embodiments, the inserted sequence is a cDNA sequence. In some embodiments, the inserted sequence includes, a portion of human IL1RL2 exon 2, human IL1RL2 exons 3-8, a portion of human IL1RL2 exon 9, a portion of endogenous IL1RL2 exon 9, endogenous IL1RL2 exon 10-11, a mouse IL1RL2 3′ UTR sequence, and/or a PolyA sequence. In some embodiments, the inserted sequence does not encode a IL1RL2 signal peptide. In some embodiments, the inserted sequence encodes a human IL1RL2 extracellular region, a human IL1RL2 transmembrane region, and an endogenous IL1RL2 cytoplasmic region.

In some embodiments, the insertion described herein is between any two nucleotides within exon 1, intron 1, exon 2, intron 2, exon 3, intron 3, exon 4, intron 4, exon 5, intron 5, exon 6, intron 6, exon 7, intron 7, exon 8, intron 8, exon 9, intron 9, exon 10, intron 10, and exon 11 of endogenous IL1RL2 gene (e.g., mouse IL1RL2 gene). In some embodiments, the insertion is between any two nucleotides within exon 2 of endogenous IL1RL2 gene. For example, the insertion is between any two of the nucleotides selected from the group consisting of positions 238-240 of exon 2 of endogenous IL1RL2 gene.

In some embodiments, the genetically modified animals can express a chimeric (e.g., humanized) IL1RL2 from endogenous mouse loci, wherein a sequence encoding the extracellular region and transmembrane region of human IL1RL2, and the cytoplasmic region of endogenous IL1RL2 is inserted within exon 2 of endogenous IL1RL2 gene. In some embodiments, human portion of the chimeric (e.g., humanized) IL1RL2 comprises an amino acid sequence that is at least 80%, 90%, 95%, 96%, 97%, 98%, or 99% identical to amino acids 1-356 of SEQ ID NO: 2. In various embodiments, an endogenous non-human IL1RL2 locus is modified in whole or in part to comprise human nucleic acid sequence encoding at least the extracellular region and/or transmembrane region of human IL1RL2 protein.

In some embodiments, the genetically modified mice can express the chimeric IL1RL2 (e.g., humanized IL1RL2) from endogenous loci that are under control of mouse promoters and/or mouse regulatory elements. The insertion at the endogenous mouse loci provides non-human animals that express chimeric IL1RL2 (e.g., humanized IL1RL2) in appropriate cell types and in a manner that does not result in the potential pathologies observed in some other transgenic mice known in the art. The chimeric IL1RL2 (e.g., humanized IL1RL2) expressed in animal can maintain one or more functions of the wild-type mouse or human IL1RL2 in the animal. For example, the expressed IL1RL2 can bind to human or non-human IL36A. Furthermore, in some embodiments, the animal does not express endogenous IL1RL2. In some embodiments, the animal expresses a decreased level of endogenous IL1RL2 as compared to a wild-type animal. As used herein, the term “endogenous IL1RL2” refers to IL1RL2 protein that is expressed from an endogenous IL1RL2 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human IL1RL2 (SEQ ID NO: 2). In some embodiments, the genome comprises a sequence encoding an amino acid sequence that is at least 70%, 75%, 80%, 85%, 90%, 95%, 99%, or 100% identical to SEQ ID NO: 10.

The genome of the genetically modified animal can comprise an insertion at an endogenous IL1RL2 gene locus. In some embodiments, the sequence is inserted between two nucleotides within any sequence of the endogenous IL1RL2 gene locus, e.g., exon 1, exon 2, exon 3, exon 4, exon 5, exon 6, exon 7, exon 8, exon 9, exon 10, exon 11, 5′-UTR, 3′-UTR, intron 1, intron 2, intron 3, intron 4, intron 5, intron 6, intron 7, intron 8, intron 9, intron 10. In some embodiments, the sequence is inserted within the regulatory region of the endogenous IL1RL2 gene. In some embodiments, the sequence is inserted within exon 1 or exon 2 of an endogenous mouse IL1RL2 gene locus.

The genetically modified animal can have one or more cells expressing a human or chimeric IL1RL2 (e.g., humanized IL1RL2) having, from N-terminus to C-terminus, a signal peptide, an extracellular region, a transmembrane region, and a cytoplasmic region. In some embodiments, the extracellular region comprises a sequence that is at least 50%, 60%, 70%, 80%, 90%, 95%, 99% identical to the extracellular region of human IL1RL2 (e.g., amino acids 20-335 of SEQ ID NO: 2). In some embodiments, the extracellular region of the humanized IL1RL2 has a sequence that has at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 311, 312, 313, 314, 315 or 316 amino acids (e.g., contiguously or non-contiguously) that are identical to the extracellular region of human IL1RL2. Because human IL1RL2 and non-human IL1RL2 (e.g., mouse IL1RL2) sequences, in many cases, are different, antibodies that bind to human IL1RL2 will not necessarily have the same binding affinity with non-human IL1RL2 or have the same effects to non-human IL1RL2. Therefore, the genetically modified animal having a human or a humanized extracellular region can be used to better evaluate the effects of anti-human IL1RL2 antibodies in an animal model.

In some embodiments, the transmembrane region comprises a sequence that is at least 50%, 60%, 70%, 80%, 90%, 95%, 99% identical to the transmembrane region of human IL1RL2 (e.g., amino acids 336-356 of SEQ ID NO: 2). In some embodiments, the transmembrane region of the humanized IL1RL2 has a sequence that has at least 1, 2, 3, 4, 5, 6, 7, 8, 9, 10, 11, 12, 13, 14, 15, 16, 17, 18, 19, 20, or 21 amino acids (contiguously or non-contiguously) that are identical to the transmembrane region of human IL1RL2.

In some embodiments, the signal peptide comprises a sequence that is at least 50%, 60%, 70%, 80%, 90%, 95%, 99% identical to the signal peptide of human IL1RL2 (e.g., amino acids 1-19 of SEQ ID NO: 2). In some embodiments, the signal peptide of the humanized IL1RL2 has a sequence that has at least 1, 2, 3, 4, 5, 6, 7, 8, 9, 10, 11, 12, 13, 14, 15, 16, 17, 18, or 19 amino acids (contiguously or non-contiguously) that are identical to the signal peptide of human IL1RL2.

In some embodiments, the cytoplasmic comprises a sequence that is at least 50%, 60%, 70%, 80%, 90%, 95%, 99% identical to the cytoplasmic of endogenous IL1RL2 (e.g., amino acids 360-574 of SEQ ID NO: 1). In some embodiments, the cytoplasmic region of the humanized IL1RL2 has a sequence that has at least 10, 20, 30, 40, 50, 60, 70, 80, 90, 100, 110, 120, 130, 140, 150, 160, 170, 180, 190, 200, 210, 211, 212, 213, 214, or 215 amino acids (contiguously or non-contiguously) that are identical to the cytoplasmic region of endogenous IL1RL2. In some embodiments, the entire extracellular region (including the signal peptide) and transmembrane region of the humanized IL1RL2 described herein are derived from human sequence. In some embodiments, the entire cytoplasmic region of the humanized IL1RL2 described herein are derived from endogenous sequence (e.g., mouse sequence).

In some embodiments, the genome of the genetically modified animal comprises a sequence that corresponds to a portion or the entire sequence of exon 1, exon 2, exon 3, exon 4, exon 5, exon 6, exon 7, exon 8, and/or exon 9 of human IL1RL2 gene; a sequence encoding the extracellular region and the transmembrane region of human IL1RL2; or a portion or the entire sequence of SEQ ID NO: 5.

In some embodiments, the genome of the genetically modified animal comprises a portion of exon 2, exons 3-8, and a portion of exon 9 of human IL1RL2 gene. In some embodiments, the portion of exon 2 includes at least 5, 10, 15, 20, 25, 30, 35, 40, 45, 50, 55, 56, 57 or 58 nucleotides. In some embodiments, the portion of exon 2 includes 57 or 58 nucleotides. In some embodiments, the portion of exon 2 includes a nucleotide sequence of at least 20 bp. In some embodiments, the portion of exon 2 starts from any one of the nucleotides encoding the N-terminal 1-5 (e.g., 1, 2, 3, 4, or 5) amino acids of IL1RL2 extracellular region (including the signal peptide) and ends at the last nucleotide of exon 2. In some embodiments, the portion of exon 9 includes at least 10, 20, 30, 40, 50, 60, 65, 66, 67, 68, 69, 70, 71, 72, 73, 74, 75, 76, or 77 nucleotides. In some embodiments, the portion of exon 9 includes 67 nucleotides. In some embodiments, the portion of exon 9 includes a nucleotide sequence of at least 50 bp. In some embodiments, the human sequence encodes the entire extracellular region and transmembrane region of human IL1RL2. In some embodiments, the human sequence further encodes the N-terminal 1, 2, 3, or 4 amino acids of the extracellular region.

In some embodiments, the non-human animal can have, at an endogenous IL1RL2 gene locus, a nucleotide sequence encoding a chimeric human/non-human IL1RL2 polypeptide, wherein a human portion of the chimeric human/non-human IL1RL2 polypeptide comprises the entire human IL1RL2 extracellular domain and the entire human IL1RL2 transmembrane region, and wherein the animal expresses a functional IL1RL2 on a surface of a cell (e.g., dendritic cell, CD4+ and CD8+ T cell) of the animal. The human portion of the chimeric human/non-human IL1RL2 polypeptide can comprise an amino acid sequence encoded by a portion of exon 2, exons 3-8, and/or a portion of exon 9 of human IL1RL2 gene. In some embodiments, the human portion of the chimeric human/non-human IL1RL2 polypeptide comprises a sequence that is at least 80%, 85%, 90%, 95%, or 99% identical to amino acids 1-356 of SEQ ID NO: 2. In some embodiments, the cytoplasmic region includes a sequence corresponding to the entire or part of amino acids 360-574 of SEQ ID NO: 1. In some embodiments, the chimeric human/non-human IL1RL2 polypeptide comprises a signal peptide, which includes a sequence corresponding to the entire or part of amino acids 1-19 of SEQ ID NO: 2.

In some embodiments, the non-human portion of the chimeric human/non-human IL1RL2 polypeptide comprises the entire cytoplasmic region of an endogenous non-human IL1RL2 polypeptide.

Furthermore, the genetically modified animal can be heterozygous with respect to the insertion at the endogenous IL1RL2 locus, or homozygous with respect to the insertion at the endogenous IL1RL2 locus.

In some embodiments, the humanized IL1RL2 locus lacks a human IL1RL2 5′-UTR. In some embodiment, the humanized IL1RL2 locus comprises an endogenous (e.g., mouse) 5′-UTR. In some embodiments, the humanization comprises an endogenous (e.g., mouse) 3′-UTR. In appropriate cases, it may be reasonable to presume that the mouse and human IL1RL2 genes appear to be similarly regulated based on the similarity of their 5′-flanking sequence. As shown in the present disclosure, humanized IL1RL2 mice that comprise an insertion at an endogenous mouse IL1RL2 locus, which retain mouse regulatory elements but comprise a humanization of IL1RL2 encoding sequence, do not exhibit pathologies. Both genetically modified mice that are heterozygous or homozygous for humanized IL1RL2 are grossly normal.

In another aspect, the disclosure also provides a genetically-modified, non-human animal whose genome comprise a disruption in the animal's endogenous IL1RL2 gene, wherein the disruption of the endogenous IL1RL2 gene comprises deletion of exon 1, exon 2, exon 3, exon 4, exon 5, exon 6, exon 7, exon 8, exon 9, exon 10, and/or exon 11, or part thereof of the endogenous IL1RL2 gene.

In some embodiments, the disruption of the endogenous IL1RL2 gene comprises deletion of one or more exons or part of exons selected from the group consisting of exon 1, exon 2, exon 3, exon 4, exon 5, exon 6, exon 7, exon 8, exon 9, exon 10, and exon 11 the endogenous IL1RL2 gene.

In some embodiments, the disruption of the endogenous IL1RL2 gene further comprises deletion of one or more introns or part of introns selected from the group consisting of intron 1, intron 2, intron 3, intron 4, intron 5, intron 6, intron 7, intron 8, intron 9, and intron 10 of the endogenous IL1RL2 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 2000, 3000, 4000, or more nucleotides.

In some embodiments, the disruption of the endogenous IL1RL2 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, exon 7, exon 8, exon 9, exon 10, and exon 11.

In some embodiments, the disruption of the endogenous IL1RL2 gene is caused by insertion of a sequence including one or more auxiliary sequences (e.g., WPRE, lox2, STOP, and/or polyA). The insertion can cause frameshift, mutation, or truncation of the endogenous IL1RL2 coding sequence, such that the level of transcription and/or translation of endogenous IL1RL2 gene is decreased (e.g., by at least 50%, at least 60%, at least 70%, at least 80%, at least 90%, at least 95%, or at least 99%).

The disclosure further relates to a IL1RL2 genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

Thus, in some embodiments, the present disclosure also provides a chimeric (e.g., humanized) IL1RL2 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL1RL2 mRNA sequence, mouse IL1RL2 amino acid sequence (e.g., SEQ ID NO: 1), or a portion thereof (e.g., a portion of exon 9, and exons 10-11); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL1RL2 mRNA sequence, human IL1RL2 amino acid sequence (e.g., SEQ ID NO: 2), or a portion thereof (e.g., a portion of exon 2, exons 3-8, and a portion of exon 9).

In some embodiments, the nucleic acids as described herein are operably linked to a promotor or regulatory element, e.g., an endogenous mouse IL1RL2 promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse IL1RL2 nucleotide sequence (e.g., a portion of exon 9, and exons 10-11).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human IL1RL2 nucleotide sequence (e.g., a portion of exon 2, exons 3-8, and a portion of exon 9).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse IL1RL2 amino acid sequence (e.g., amino acids 360-574 of SEQ ID NO: 1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human IL1RL2 amino acid sequence (e.g., amino acids 1-356 of SEQ ID NO: 2).

The present disclosure also provides a humanized IL1RL2 mouse amino acid sequence, wherein the amino acid sequence is selected from the group consisting of:
a) an amino acid sequence shown in SEQ ID NO: 1, 2 or 10;
b) an amino acid sequence having a homology of at least 90% with or at least 90% identical to the amino acid sequence shown in SEQ ID NO: 1, 2, or 10;
c) an amino acid sequence encoded by a nucleic acid sequence, wherein the nucleic acid sequence is able to hybridize to a nucleotide sequence encoding the amino acid shown in SEQ ID NO: 1, 2, or 10 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 1, 2, or 10;
e) an amino acid sequence that is different from the amino acid sequence shown in SEQ ID NO: 1, 2, or 10 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 1, 2, or 10.

The present disclosure also provides a humanized IL1RL2 amino acid sequence, wherein the amino acid sequence is selected from the group consisting of:
a) all or part of amino acids 1-356 of SEQ ID NO: 2;
b) an amino acid sequence have a homology of at least 90%, 91%, 92%, 93%, 94%, 95%, 96%, 97%, 98%, or 99% to amino acids 1-356 of SEQ ID NO: 2;
c) an amino acid sequence that is different from amino acids 1-356 of SEQ ID NO: 2 by no more than 10, 9, 8, 7, 6, 5, 4, 3, 2 or no more than 1 amino acid; and
d) an amino acid sequence that comprises a substitution, a deletion and/or insertion of one or more amino acids to amino acids 1-356 of SEQ ID NO: 2.

The present disclosure also relates to a IL1RL2 nucleic acid (e.g., DNA or RNA) sequence, wherein the nucleic acid sequence can be selected from the group consisting of:
a) a nucleic acid sequence as shown in SEQ ID NO: 3, 4, 5, 6, 7, 8, 9, 11, or 12, or a nucleic acid sequence encoding a homologous IL1RL2 amino acid sequence of a humanized mouse IL1RL2;
b) a nucleic acid sequence that is able to hybridize to the nucleotide sequence as shown in SEQ ID NO: 3, 4, 5, 6, 7, 8, 9, 11, or 12, under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 3, 4, 5, 6, 7, 8, 9, 11, or 12;
d) a nucleic acid sequence that encodes an amino acid sequence, wherein the amino acid sequence has a homology of at least 90% with or at least 90% identical to the amino acid sequence shown in SEQ ID NO: 1, 2, or 10;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 1, 2, or 10;
f) a nucleic acid sequence that encodes an amino acid sequence, wherein the amino acid sequence is different from the amino acid sequence shown in SEQ ID NO: 1, 2, or 10 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 1, 2, or 10.

The present disclosure further relates to a IL1RL2 genomic DNA sequence of a humanized mouse. The DNA sequence is obtained by reverse transcription of the mRNA obtained by transcription thereof is consistent with or complementary to the DNA sequence homologous to the sequence shown in SEQ ID NO: 5 or 6.

### IL36A

The IL-36 family belongs to a larger IL-1 superfamily and consists of three agonists (IL-36α/β/γ), one antagonist (IL-36Ra), one cognate receptor (IL-36R) and one accessory protein (IL-1RAcP). The receptor activation follows a two-step mechanism in that the agonist first binds to IL-36R and the resulting binary complex recruits IL-1RAcP. Assembled ternary complex brings together intracellular TIR domains of receptors which activate downstream NF-κB and MAPK signaling. Members of IL-36 are normally expressed at low levels. Upon stimulation, they are inducted and act on a variety of cells including epithelial and immune cells. Protease mediated N-terminal processing is needed for cytokine activation. In the skin, the functional role of IL-36 is to contribute to host defense through inflammatory response. However, when dysregulated, IL-36 stimulates keratinocyte and immune cells to enhance the Th17/Th23 axis and induces psoriatic-like skin disorder. Genetic mutations of the antagonist IL-36Ra are associated with occurrence of generalized pustular psoriasis, a rare but life-threatening skin disease. Anti-IL-36 antibodies attenuate IMQ or IL-23 induced skin inflammation in mice, illustrating IL-36's involvement in mouse model of psoriasis. Other organs such as the lungs, the intestine, the joints and the brain also express IL-36 family members upon stimulation.

The expression of IL-36 cytokines and IL-36 receptor (IL-1R6) has been described in many different tissues. The underlying expression profile is more limited compared to that of the “traditional” IL-1 cytokines. IL-1R6 is mainly found on epithelial cells at the barrier sites of an organism. The IL-36 isoforms IL-36α, IL-36β, IL-36γ, and IL-36 receptor antagonist (IL-36 Ra) are predominantly produced in the skin by keratinocytes. Furthermore, the isoforms IL-36α and IL-36γ are expressed in the respiratory tract and IL-36β as well as IL-36γ are expressed in the intestines. The presence of IL-1R6 and the activity of the isoforms IL-36γ and IL-36 Ra was shown in murine glial cells, suggesting a role of IL-36 axis in brain physiology. It is also known that immune cells such as plasma cells, T-cells, macrophages and dendritic cells (DC) produce IL-36 under certain conditions, such as inflammation due to pathological changes. Compared to wild type mice, there was a stronger, no self-limiting skin inflammation in immunoregulator Regnase 1 (Reg-1) knockout mice with higher IL-36α levels.

The IL-36 receptor, also known as Interleukin-1 Receptor-Related Protein 2 (IL-1Rrp2) or Interleukin 1 Receptor Like 2 (IL1RL2) was finally assigned as IL-1R6. Its ligands include all members of the IL-36 family: IL-36α, IL-36β, IL-36γ, and IL-36 Ra.

Members of the IL-36 family are thought to have an important role in bridging the innate and adaptive immune systems. They do not only recruit and activate cells of the innate immune system, but they also have indirect and direct effects on the proliferation and plasticity of adaptive immune cells. It was shown, that IL-36 signaling appears to have a beneficial effect on T-cell proliferation. Furthermore, it helps polarizing naïve T-helper cells toward T-helper 1 cells in an IL-2 mediated fashion. Mice lacking IL-1R6 showed a decreased resolution of intestinal damage after treatment with dextran sodium sulfate. The pro-inflammatory function of three agonistic IL-36 isoforms is opposed by IL-36 Ra, which has anti-inflammatory properties as a “natural inhibitor.”

A detailed description of IL36A and its function can be found, e.g., in Buhl, Anna-Lena, and Joerg Wenzel. “Interleukin-36 in infectious and inflammatory skin diseases.” Frontiers in Immunology 10 (2019): 1162; Zhou, Li, and Viktor Todorovic. “Interleukin-36: structure, signaling and function.” Protein Reviews (2020): 191-210, each of which is incorporated by reference in its entirety.

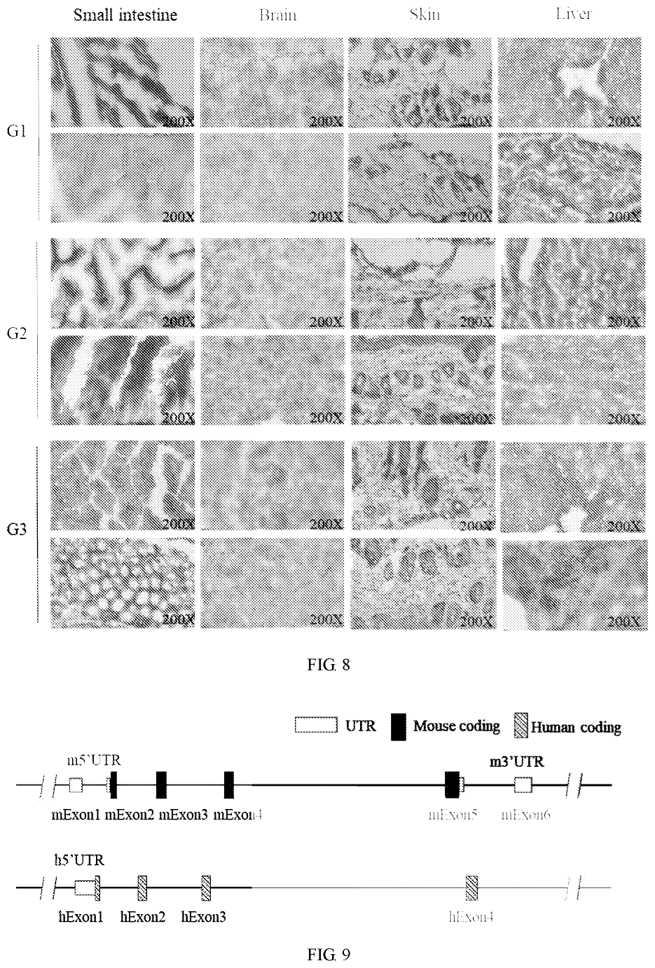

In human genomes, IL36A gene (Gene ID: 27179) locus has four exons, exon 1, exon 2, exon 3 and exon 4 (FIG. 9). The IL36A protein also has a propeptide, The nucleotide sequence for human IL36A mRNA is NM_014440.3, and the amino acid sequence for human IL36A is NP_055255.1 (SEQ ID NO: 30). The location for each exon and each region in human IL36A nucleotide sequence and amino acid sequence is listed below:

TABLE 3

| | | NP_055255.1 |
|---|---|---|
| Human IL36A | NM_014440.3 | 158aa |
| (approximate location) | 890 bp | (SEQ ID NO: 30) |
| Exon 1 | 1-423 | 1-3 |
| Exon 2 | 424-537 | 4-41 |
| Exon 3 | 538-677 | 42-88 |
| Exon 4 | 678-890 | 89-158 |
| propeptide | 414-428 | 1-5 |
| Chain | 429-887 | 6-158 |
| Donor region in Example | 414-890 | 1-158+ stop codon |

The humanIL36A gene (Gene ID: 27179) is located in Chromosome 2 of the human genome, which is located from 113005459 to 113011071 of NC_000002.12 (GRCh38.p13 (GCF_000001405.39)). The 5′-UTR is from 113,005,459 to 113,005,871, exon 1 is from 113,005,459 to 113,005,881, the 1st intron is from 113,005,882 to 113,005,973, exon 2 is from 113,005,974 to 113,006,087, the 2th intron is from 113,006,088 to 113,006,597, exon 3 is from 113,006,598 to 113,006,737, the 3th intron is from 113,006,738 to 113,007,831, and exon 4 is from 113,007,832 to 113,008,044, based on transcript NM_014440.3. All relevant information for humanIL36A locus can be found in the NCBI website with Gene ID: 27179, which is incorporated by reference herein in its entirety.

In mice, IL36A gene locus has six exons, exon 1, exon 2, exon 3, exon 4, exon 5, and exon 6 (FIG. 9). The mouse IL36A protein also has a propeptide. The nucleotide sequence for mouse IL36A mRNA is NM_019450.3, the amino acid sequence for mouse IL36A is NP_062323.1 (SEQ ID NO: 29). The location for each exon and each region in the mouse IL36A nucleotide sequence and amino acid sequence is listed below:

TABLE 4

| | | NP 062323.1 |
|---|---|---|
| Mouse IL36A | NM_019450.3 | 160 aa |
| (approximate location) | 883 bp | (SEQ ID NO: 29) |
| Exon 1 | 1-119 | non-coding |
| Exon 2 | 120-177 | 1-5 |
| Exon 3 | 178-291 | 6-43 |
| Exon 4 | 292-431 | 44-90 |
| Exon 5 | 432-645 | 91-160 |
| Exon 6 | 646-883 | non-coding |
| propeptide | 162-182 | 1-7 |
| chain | 183-641 | 8-160 |
| Replaced region in Example | 162-641 | 1-160 |

The mouse IL36A gene (Gene ID: 54448) is located in Chromosome 2 of the mouse genome, which is located from 24215417 to 24225701 of NC_000068.7 (GRCm38.p6 (GCF_000001635.26)). The 5′-UTR is from 24,215,418 to U.S. Pat. Nos. 24,215,535 and 24,215,805 to 24,215,846, exon 1 is from 24,215,418 to 24,215,535, the first intron is from 24,215,536 to 24,215,804, exon 2 is from 24,215,805 to 24,215,862, the second intron is from 24,215,863 to 24,215,950, exon 3 is from 24,215,951 to 24,216,064, the third intron is from 24,216,065 to 24,216,535, exon 4 is from 24,216,536 to 24,216,675, the forth intron is from 24,216,676 to 24,224,384, exon 5 is from 24,224,385 to 24,224,598, the fifth intron is from 24,224,599 to 24,225,463, exon 6 is from 24,225,464 to 24,225,702, the 3′-UTR is from U.S. Pat. Nos. 24,224,598 and 24,225,464 to 24,225,702, based on transcriptNM_019450.3. All relevant information for mouseIL36A locus can be found in the NCBI website with Gene ID: 54448, which is incorporated by reference herein in its entirety.

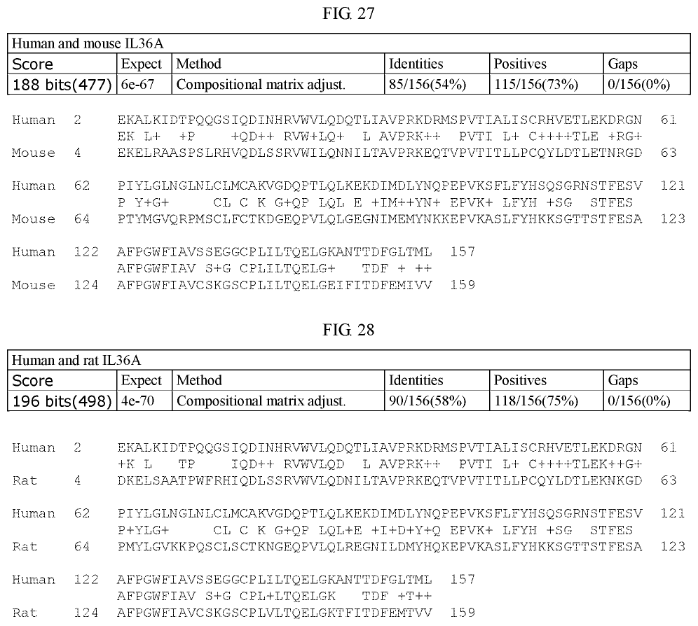

FIG. 27 shows the alignment between human IL36A amino acid sequence (NP_055255.1; SEQ ID NO: 30) and mouse IL36A amino acid sequence (NP_062323.1; SEQ ID NO: 29). Thus, the corresponding amino acid residue or region between human and mouse IL36A can be found in FIG. 27.

IL36A genes, proteins, and locus of the other species are also known in the art. For example, the gene ID for IL36A in Rattus norvegicus (rat) is 296541, the gene ID for IL36A in Macaca mulatta (Rhesus monkey) is 705136, and the gene ID for IL36A in Oryctolagus cuniculus (rabbit) is 100355520. The relevant information for these genes (e.g., intron sequences, exon sequences, amino acid residues of these proteins) can be found, e.g., in NCBI database, which is incorporated by reference herein in its entirety. FIG. 28 shows the alignment between human IL36A amino acid sequence (NP_055255.1; SEQ ID NO: 30) and rat IL36A amino acid sequence (NP_001100024.1; SEQ ID NO: 81). Thus, the corresponding amino acid residue or region between human and rodent IL36A can be found in FIG. 28.

The present disclosure provides human or chimeric (e.g., humanized) IL36A nucleotide sequence and/or amino acid sequences. In some embodiments, the entire sequence of mouse exon 1, exon 2, exon 3, exon 4, exon 5 and/or exon 6 are replaced by the corresponding human sequence. In some embodiments, a “region” or “portion” of mouse exon 1, exon 2, exon 3, exon 4, exon 5 and/or exon 6 are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450 460, 470, 480, 490, 500, 550, 600, 650, 700, 750, 760, 770, 780, 790, 800, 810, 820, 830, 840, 850, 860, 870, 880, or 883 nucleotides, or at least 1, 2, 3, 4, 5, 6, 7, 8, 9, 10, 20, 30, 40, 50, 60, 70, 80, 90, 100, 110, 120, 130, 140, 150, 155, 156, 157, 158, 159, or 160 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5 and/or exon 6. In some embodiments, a region, a portion, or the entire sequence of mouse exon 1, exon 2, exon 3, exon 4, exon 5 and/or exon 6 (e.g., a portion of exon 2, exons 3-4, and a portion of exon 5) are replaced by a region, a portion, or the entire sequence of the human exon 1, exon 2, exon 3, and/or exon 4 (e.g., a portion of exon 1, exons 2-4).

In some embodiments, a “region” or “portion” of endogenous exon 1, exon 2, exon 3, exon 4, exon 5 and/or exon 6 is deleted.

In some embodiments, the present disclosure is related to a genetically-modified, non-human animal whose genome comprises a human, chimeric, or humanized IL36A nucleotide sequence. In some embodiments, the human, chimeric, or humanized IL36A nucleotide sequence encodes a IL36A protein that is at least 80%, 85%, 90%, 95%, or 100% identical to SEQ ID NO: 30. In some embodiments, the genome of the animal comprises a sequence that is at least 80%, 85%, 90%, 95%, or 100% identical to SEQ ID NO: 31, 32, 33, 34, 36, 37, 38, or 39.

In some embodiments, the genetically-modified non-human animal described herein comprises a sequence encoding a human or humanized IL36A protein. In some embodiments, the humanized IL36A protein comprises a human or humanized propeptide. In some embodiments, the humanized IL36A protein comprises an endogenous propeptide.

In some embodiments, the genetically-modified non-human animal described herein comprises a human or humanized IL36A gene. In some embodiments, the humanized IL36A gene comprises 4 exons. In some embodiments, the humanized IL36A gene comprises humanized exon 1, human exon 2, human exon 3, and/or humanized exon 4. In some embodiments, the humanized IL36A gene comprises 3 introns. In some embodiments, the humanized IL36A gene comprises human intron 1, human intron 2, and/or human intron 3. In some embodiments, the humanized IL36A gene comprises human or humanized 5′ UTR. In some embodiments, the humanized IL36A gene comprises human or humanized 3′ UTR. In some embodiments, the humanized IL36A gene comprises endogenous 5′ UTR. In some embodiments, the humanized IL36A gene comprises endogenous 3′ UTR.

In some embodiments, the genetically modified animals can express a human IL36A and/or a chimeric (e.g., humanized) IL36A from endogenous mouse loci, wherein the endogenous mouse IL36A gene has been replaced with a human IL36A gene and/or a nucleotide sequence that encodes a region of human IL36A sequence or an amino acid sequence that is at least 10%, 20%, 30%, 40%, 50%, 60%, 70&, 80%, 90%, 95%, 96%, 97%, 98%, or 99% identical to the human IL36A sequence. In various embodiments, an endogenous non-human IL36A locus is modified in whole or in part to comprise human nucleic acid sequence encoding at least one protein-coding sequence of a mature IL36A protein.

In some embodiments, the genetically modified mice can express the human IL36A and/or chimeric IL36A (e.g., humanized IL36A) from endogenous loci that are under control of mouse promoters and/or mouse regulatory elements. The replacement(s) at the endogenous mouse loci provide non-human animals that express human IL36A or chimeric IL36A (e.g., humanized IL36A) in appropriate cell types and in a manner that does not result in the potential pathologies observed in some other transgenic mice known in the art. The human IL36A or the chimeric IL36A (e.g., humanized IL36A) expressed in animal can maintain one or more functions of the wild-type mouse or human IL36A in the animal. For example, the expressed IL36A can bind to human or non-human IL36R. Furthermore, in some embodiments, the animal does not express endogenous IL36A. In some embodiments, the animal expresses a decreased level of endogenous IL36A as compared to a wild-type animal. As used herein, the term “endogenous IL36A” refers to IL36A protein that is expressed from an endogenous IL36A nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human IL36A (SEQ ID NO: 30). In some embodiments, the genome comprises a sequence that is at least 70%, 75%, 80%, 85%, 90%, 95%, 99%, or 100% identical to SEQ ID NO: 33.

The genome of the genetically modified animal can comprise a replacement at an endogenous IL36A gene locus of a sequence encoding a region of endogenous IL36A with a sequence encoding a corresponding region of human IL36A. In some embodiments, the sequence that is replaced is any sequence within the endogenous IL36A gene locus, e.g., exon 1, exon 2, exon 3, exon 4, exon 5, exon 6, 5′-UTR, 3′-UTR, intron 1, intron 2, intron 3, intron 4, intron 5 or any combination thereof. In some embodiments, the sequence that is replaced is within the regulatory region of the endogenous IL36A gene. In some embodiments, the sequence that is replaced is exon 1, exon 2, exon 3, exon 4, exon 5 and/or exon 6, or a portion thereof, of an endogenous mouse IL36A gene locus.

The genetically modified animal can have one or more cells expressing a human or chimeric IL36A (e.g., humanized IL36A). In some embodiments, the human or chimeric IL36A has a sequence that has at least 10, 20, 30, 40, 50, 60, 70, 80, 90, 100, 110, 120, 130, 140, 150, 155, 156, 157, 158, or 159 amino acids (e.g., contiguously or non-contiguously) that are identical to human IL36A (e.g., SEQ ID NO: 30).

In some embodiments, the genome of the genetically modified animal comprises a sequence that corresponds to a portion or the entire sequence of exon 1, exon 2, exon 3, and/or exon 4 of human IL36A gene; a portion or the entire sequence of human IL36A gene; or a portion or the entire sequence of SEQ ID NO: 30.

In some embodiments, the genome of the genetically modified animal comprises a portion of exon 1, exons 2-3, and a portion or the entirety of exon 4 of human IL36A gene. In some embodiments, the portion of exon 1 includes at least 5, 6, 7, 8, 9 or 10 nucleotides. In some embodiments, the portion of exon 1 includes 9 or 10 nucleotides. In some embodiments, the portion of exon 1 includes a nucleotide of at least 2 bp. In some embodiments, the portion of exon 4 includes at least 10, 20, 30, 40, 50, 60, 70, 80, 90, 100, 110, 120, 121, 122, 123, 124, 125, 126, 127, 128, 129, 130, 150, 160, 170, 180, 190, 200, 210, 211, 212 or 213 nucleotides. In some embodiments, the portion of exon 4 includes 212 or 213 nucleotides. In some embodiments, the portion of exon 5 includes a nucleotide of at least 150 bp or at least 200 bp. In some embodiments, the replaced sequence encodes the coding sequence of human IL36A (e.g., positions 414-890 of NM_014440.3).

Furthermore, the genetically modified animal can be heterozygous with respect to the replacement at the endogenous IL36A locus, or homozygous with respect to the replacement at the endogenous IL36A locus.

In some embodiments, the humanized IL36A locus lacks a human IL36A 5′-UTR. In some embodiment, the humanized IL36A locus comprises an endogenous (e.g., mouse) 5′-UTR. In some embodiments, the humanization comprises an endogenous (e.g., mouse) 3′-UTR. In appropriate cases, it may be reasonable to presume that the mouse and human IL36A genes appear to be similarly regulated based on the similarity of their 5′-flanking sequence. As shown in the present disclosure, humanized IL36A mice that comprise a replacement at an endogenous mouse IL36A locus, which retain mouse regulatory elements but comprise a humanization of IL36A encoding sequence, do not exhibit pathologies. Both genetically modified mice that are heterozygous or homozygous for humanized IL36A are grossly normal.

In another aspect, the disclosure also provides a genetically-modified, non-human animal whose genome comprise a disruption in the animal's endogenous IL36A gene, wherein the disruption of the endogenous IL36A gene comprises deletion of exon 1, exon 2, exon 3, exon 4, exon 5 and/or exon 6, or part thereof of the endogenous IL36A gene.

In some embodiments, the disruption of the endogenous IL36A gene comprises deletion of one or more exons or part of exons selected from the group consisting of exon 1, exon 2, exon 3, exon 4, exon 5 and exon 6 of the endogenous IL36A gene.

In some embodiments, the disruption of the endogenous IL36A gene further comprises deletion of one or more introns or part of introns selected from the group consisting of exon 2, intron 2, exon 3, intron 3, intron 4, exon 4 and exon 5 of the endogenous IL36A gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, or more nucleotides.

In some embodiments, the disruption of the endogenous IL36A gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 650, 700, 750, 800, 850, 860, 870, 880, or 883 nucleotides of exon 1, exon 2, exon 3, exon 4, exon 5 and/or exon 6 (e.g., deletion of a portion of exon 2, exons 3-4, and a portion of exon 5).

The disclosure further relates to a IL36A genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

Thus, in some embodiments, the present disclosure also provides a chimeric (e.g., humanized) IL36A nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL36A mRNA sequence, mouse IL36A amino acid sequence (e.g., SEQ ID NO: 29), or a portion thereof (e.g., 5′ UTR, a portion of exon 1, a portion of exon 5, exon 6, and 3′ UTR); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL36A mRNA sequence, human IL36A amino acid sequence (e.g., SEQ ID NO: 30), or a portion thereof (e.g., a portion of exon 1, and exons 2-4).

In some embodiments, the sequence encoding amino acids 1-160 of mouse IL36A (SEQ ID NO: 29) is replaced. In some embodiments, the sequence is replaced by a sequence encoding a corresponding region of human IL36A (e.g., amino acids 1-158 of human IL36A (SEQ ID NO: 30))

In some embodiments, the nucleic acids as described herein are operably linked to a promotor or regulatory element, e.g., an endogenous mouse IL36A promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse IL36A nucleotide sequence (e.g., a portion of exon 2, exons 3-4, and a portion of exon 5).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse IL36A nucleotide sequence (e.g., 5′ UTR, exon 1, a portion of exon 2, a portion of exon 5, exon 6, and 3′ UTR).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human IL36A nucleotide sequence (e.g., 5′ UTR, a portion of exon 1, and 3′ UTR).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human IL36A nucleotide sequence (e.g., a portion of exon 1, and exons 2-4).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse IL36A amino acid sequence (e.g., SEQ ID NO: 29).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse IL36A amino acid sequence (e.g., SEQ ID NO: 29).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human IL36A amino acid sequence (e.g., SEQ ID NO: 30).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human IL36A amino acid sequence (e.g., SEQ ID NO: 30).

The present disclosure also provides a humanized IL36A mouse amino acid sequence, wherein the amino acid sequence is selected from the group consisting of:
a) an amino acid sequence shown in SEQ ID NO: 30;
b) an amino acid sequence having a homology of at least 90% with or at least 90% identical to the amino acid sequence shown in SEQ ID NO: 30;
c) an amino acid sequence encoded by a nucleic acid sequence, wherein the nucleic acid sequence is able to hybridize to a nucleotide sequence encoding the amino acid shown in SEQ ID NO: 30 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 30;
e) an amino acid sequence that is different from the amino acid sequence shown in SEQ ID NO: 30 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 30.

The present disclosure also relates to a IL36A nucleic acid (e.g., DNA or RNA) sequence, wherein the nucleic acid sequence can be selected from the group consisting of:
a) a nucleic acid sequence as shown in SEQ ID NO: 31, 32, 33, 34, 35, 36, 37, 38, or 39, or a nucleic acid sequence encoding a homologous IL36A amino acid sequence of a humanized mouse IL36A;
b) a nucleic acid sequence that is able to hybridize to the nucleotide sequence as shown in SEQ ID NO: 31, 32, 33, 34, 35, 36, 37, 38, or 39 under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 31, 32, 33, 34, 35, 36, 37, 38, or 39;
d) a nucleic acid sequence that encodes an amino acid sequence, wherein the amino acid sequence has a homology of at least 90% with or at least 90% identical to the amino acid sequence shown in SEQ ID NO: 30;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 30;
f) a nucleic acid sequence that encodes an amino acid sequence, wherein the amino acid sequence is different from the amino acid sequence shown in SEQ ID NO: 30 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 30.

The present disclosure further relates to a IL36A genomic DNA sequence of a humanized mouse. The DNA sequence is obtained by reverse transcription of the mRNA obtained by transcription thereof is consistent with or complementary to the DNA sequence homologous to the sequence shown in SEQ ID NO: 33.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, or 152 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) IL1RL2 from an endogenous non-human IL1RL2 locus, and/or human or chimeric (e.g., humanized) IL36A from an endogenous non-human IL36A locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an NK cell, an antigen presenting cell, a macrophage, a dendritic cell, a germ cell, a blastocyst, or an endogenous tumor cell. In some embodiments, genetically-modified non-human animals are provided that comprise a modified endogenous IL1RL2 and/or IL36A loci that comprise an exogenous sequence (e.g., a human sequence), e.g., a replacement of one or more non-human sequences with one or more human sequences, or an insertion of one or more human and/or non-human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be e.g., a human exon or a chimeric exon.

In some embodiments, the chimeric gene or the chimeric nucleic acid is a humanized IL1RL2 gene or a humanized IL1RL2 nucleic acid. In some embodiments, at least one or more portions of the gene or the nucleic acid is from the human IL1RL2 gene, at least one or more portions of the gene or the nucleic acid is from a non-human IL1RL2 gene. In some embodiments, the gene or the nucleic acid comprises a sequence that encodes an IL1RL2 protein. The encoded IL1RL2 protein is functional or has at least one activity of the human IL1RL2 protein or the non-human IL1RL2 protein, e.g., interacting with IL36A, to induce downstream signaling pathways.

In some embodiments, the chimeric gene or the chimeric nucleic acid is a humanized IL36A gene or a humanized IL36A nucleic acid. In some embodiments, at least one or more portions of the gene or the nucleic acid is from the human IL36A gene, at least one or more portions of the gene or the nucleic acid is from a non-human IL36A gene. In some embodiments, the gene or the nucleic acid comprises a sequence that encodes an IL36A protein. The encoded IL36A protein is functional or has at least one activity of the human IL36A protein or the non-human IL36A protein, e.g., interacting with IL1RL2 to induce downstream signaling pathways.

In some embodiments, the chimeric protein or the chimeric polypeptide is a humanized IL1RL2 protein or a humanized IL1RL2 polypeptide. In some embodiments, at least one or more portions of the amino acid sequence of the protein or the polypeptide is from a human IL1RL2 protein, and at least one or more portions of the amino acid sequence of the protein or the polypeptide is from a non-human IL1RL2 protein. The humanized IL1RL2 protein or the humanized IL1RL2 polypeptide is functional or has at least one activity of the human IL1RL2 protein or the non-human IL1RL2 protein.

In some embodiments, the chimeric protein or the chimeric polypeptide is a humanized IL36A protein or a humanized IL36A polypeptide. In some embodiments, at least one or more portions of the amino acid sequence of the protein or the polypeptide is from a human IL36A protein, and at least one or more portions of the amino acid sequence of the protein or the polypeptide is from a non-human IL36A protein. The humanized IL36A protein or the humanized IL36A polypeptide is functional or has at least one activity of the human IL36A protein or the non-human IL36A protein.

In some embodiments, the extracellular region described herein is human or humanized. In some embodiments, the transmembrane region described herein is human or humanized. In some embodiments, the cytoplasmic region described herein is human or humanized.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10:836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129). In some embodiments, the non-human animal is a rodent. In some embodiments, the non-human animal is a mouse having a BALB/c, A, A/He, A/J, A/WySN, AKR, AKR/A, AKR/J, AKR/N, TA1, TA2, RF, SWR, C3H, C57BR, SJL, C57L, DBA/2, KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL (C57BL/10Cr and C57BL/Ola), C58, CBA/Br, CBA/Ca, CBA/J, CBA/st, or CBA/H background.

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized IL1RL2 and/or IL36A animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γc^{null} mice (Ito, M. et al., NOD/SCID/γc^{null} mouse: an excellent recipient mouse model for engraftment of human cells, Blood 100 (9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human IL1RL2 and/or IL36A loci, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc^{null} mice, nude mice, Rag1 and/or Rag2 knockout mice, NOD-Prkdc^{scid} IL-2rγ^{null} mice, NOD-Rag 1−/−-IL2rg^{-/-} (NRG) mice, Rag 2^{-/-}-IL2rg^{-/-} (RG) mice, and a combination thereof. These genetically modified animals are described, e.g., in US20150106961, which is incorporated herein by reference in its entirety. In some embodiments, the mouse can include a replacement of all or part of mature IL1RL2 or IL36A coding sequence with human mature IL1RL2 or IL36A coding sequence. In some embodiments, the mouse can include an insertion of a chimeric (e.g., human/non-human) IL1RL2 or IL36A coding sequence at an endogenous IL1RL2 or IL36A locus.

Genetically modified non-human animals can comprise a modification at endogenous non-human IL1RL2 and/or IL36A loci. In some embodiments, the modification can comprise a human nucleic acid sequence encoding at least a portion of a mature IL1RL2 or IL36A protein (e.g., at least 10%, 20%, 30%, 40%, 50%, 60%, 70%, 80%, 90%, 95%, 96%, 97%, 98%, or 99% identical to the mature IL1RL2 or IL36A protein sequence). Although genetically modified cells are also provided that can comprise the modifications described herein (e.g., ES cells, somatic cells), in many embodiments, the genetically modified non-human animals comprise the modification of the endogenous IL1RL2 and/or IL36A loci in the germline of the animal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by humanized IL1RL2 and/or IL36A genes.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure also provides non-human mammals produced by any of the methods described herein. In some embodiments, a non-human mammal is provided; and the genetically modified animal contains the DNA encoding human or humanized IL1RL2 and/or IL36A genes in the genome of the animal.

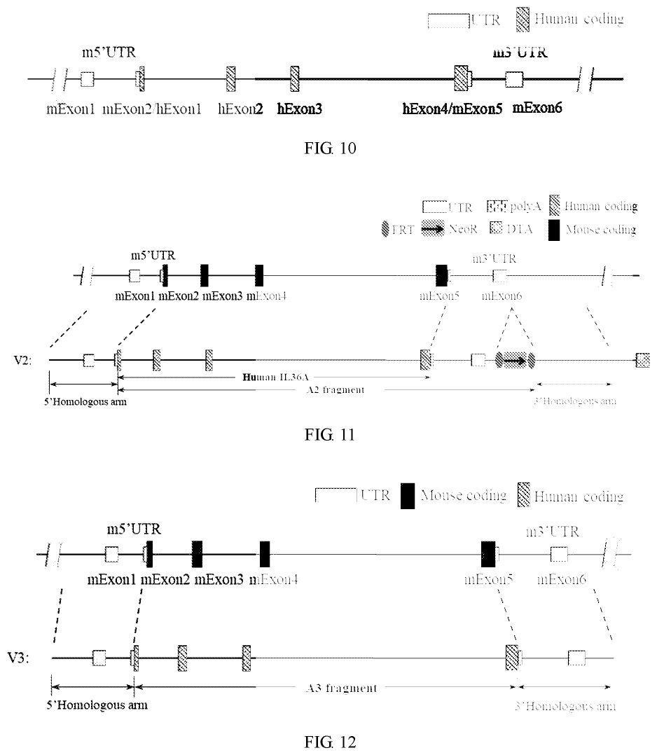

In some embodiments, the non-human mammal comprises a humanized IL1RL2 gene having the genetic construct as described herein (e.g., gene construct as shown in FIGS. 2, 3, and 5). In some embodiments, the non-human mammal comprises a humanized IL36A gene having the genetic construct as described herein (e.g., gene construct as shown in FIGS. 10-12). In some embodiments, a non-human mammal expressing human or humanized IL1RL2 and/or IL36A is provided. In some embodiments, the tissue-specific expression of human or humanized IL1RL2 and/or IL36A proteins is provided.

In some embodiments, the expression of human or humanized IL1RL2 and/or IL36A in a genetically modified animal is controllable, as by the addition of a specific inducer or repressor substance. In some embodiments, the specific inducer is selected from Tet-Off System/Tet-On System, or Tamoxifen System.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cells can be obtained from the cell culture established using the same constructs and the standard cell transfection techniques. The integration of genetic constructs containing DNA sequences encoding human IL1RL2 and/or IL36A proteins can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized IL1RL2 and/or IL36A proteins.

In some embodiments, provided herein is a genetically-modified non-human animal having two or more human or humanized genes selected from IL1RL2 and/or IL36A.

### Vectors

The present disclosure relates to a targeting vector, comprising: a) a DNA fragment homologous to the 5′ end of a region to be altered (5′ arm), which is selected from the IL1RL2 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL1RL2 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000067.6; c) the DNA fragment homologous to the 3′ end of the region to be altered (3′ arm) is selected from the nucleotide sequences that have at least 90% homology to the NCBI accession number NC_000067.6.

In some embodiments, a) the DNA fragment homologous to the 5′ end of a region to be altered (5′ arm) is selected from the nucleotides from the positions 40322546-40326494 of the NCBI accession number NC_000067.6; c) the DNA fragment homologous to the 3′ end of the region to be altered (3′ arm) is selected from the nucleotides from the positions 40326498-40330768 of the NCBI accession number NC_000067.6.

In some embodiments, the length of the selected genomic nucleotide sequence in the targeting vector can be more than about 2 kb, 2.5 kb, 3 kb, 3.5 kb, or 4 kb.

In some embodiments, the region to be altered is exon 1, exon 2, exon 3, exon 4, exon 5, exon 6, exon 7, exon 8, exon 9, exon 10 and/or exon 11 of IL1RL2 gene (e.g., a portion of exon 2 of mouse IL1RL2 gene).

The targeting vector can further include one or more selectable markers, e.g., positive or negative selectable markers. In some embodiments, the positive selectable marker is a Neo gene or Neo cassette. In some embodiments, the negative selectable marker is a DTA gene.

In some embodiments, the sequence of the 5′ arm is shown in SEQ ID NO: 3; and the sequence of the 3′ arm is shown in SEQ ID NO: 4.

In some embodiments, the desired/donor DNA sequence contains a part or entirety of exon 1, exon 2, exon 3, exon 4, exon 5, exon 6, exon 7, exon 8, exon 9, exon 10, exon 11 and/or exon 12 of the human IL1RL2 gene. In some embodiments, the desired/donor DNA sequence contains a portion of exon 2, exons 3-8 and a portion of exon 9 of the human IL1RL2 gene (e.g., nucleic acids 77-1144 of NM_003854.4).

In some embodiments, the desired/donor DNA sequence contains a part or entirety of exon 1, exon 2, exon 3, exon 4, exon 5, exon 6, exon 7, exon 8, exon 9, exon 10, and/or exon 11 of the mouse IL1RL2 gene. In some embodiments, the desired/donor DNA sequence contains a portion of exon 9, exons 10 and a portion of exon 11 of the mouse IL1RL2 gene (e.g., nucleic acids 1315-1962 of NM_133193.4).

The present disclosure relates to a targeting vector, comprising: a) a DNA fragment homologous to the 5′ end of a region to be altered (5′ arm), which is selected from the IL36A gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL36A gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000068.7; c) the DNA fragment homologous to the 3′ end of the region to be altered (3′ arm) is selected from the nucleotide sequences that have at least 90% homology to the NCBI accession number NC_000068.7.

In some embodiments, a) the DNA fragment homologous to the 5′ end of a region to be altered (5′ arm) is selected from the nucleotides from the positions 24212600-24215846 of the NCBI accession number NC_000068.7; c) the DNA fragment homologous to the 3′ end of the region to be altered (3′ arm) is selected from the nucleotides from the positions 24226002-24229603 of the NCBI accession number NC_000068.7. In some embodiments, the sequence of the 5′ arm is shown in SEQ ID NO: 31; and the sequence of the 3′ arm is shown in SEQ ID NO: 32.

In some embodiments, a) the DNA fragment homologous to the 5′ end of a region to be altered (5′ arm) is selected from the nucleotides from the positions 24213960-24215846 of the NCBI accession number NC_000068.7; c) the DNA fragment homologous to the 3′ end of the region to be altered (3′ arm) is selected from the nucleotides from the positions 24224598-24225577 of the NCBI accession number NC_000068.7. In some embodiments, the sequence of the 5′ arm is shown in SEQ ID NO: 38; and the sequence of the 3′ arm is shown in SEQ ID NO: 39.

In some embodiments, the length of the desired/donor DNA sequence in the targeting vector can be more than about 200 bp, 250 bp, 300 bp, 350 bp, 400 bp, 450 bp, 460 bp, 500 bp, 750 bp, 1000 bp, 1250 bp, 1500 bp, 1750 bp, or 2000 bp.

In some embodiments, the region to be altered is exons 2-5 of endogenous IL36A gene (e.g., nucleic acids 162-641 of mouse IL36A gene).

The targeting vector can further include one or more selectable markers, e.g., positive or negative selectable markers. In some embodiments, the positive selectable marker is a Neo gene or Neo cassette. In some embodiments, the negative selectable marker is a DTA gene.

In some embodiments, the sequence of the 5′ arm is shown in SEQ ID NO: 31; and the sequence of the 3′ arm is shown in SEQ ID NO: 32.

In some embodiments, the sequence of the 5′ arm is shown in SEQ ID NO: 38; and the sequence of the 3′ arm is shown in SEQ ID NO: 39.

In some embodiments, the desired/donor DNA sequence contains a part or entirety of exon 1, exon 2, exon 3, exon 4 of the human IL36A gene. In some embodiments, the desired/donor DNA sequence contains a portion of exon 1, and exons 2-4 of the human IL1RL2 gene (e.g., nucleic acids 414-890 of NM_014440.3).

In some embodiments, the nucleotide sequence of the humanized IL36A gene encodes a IL36A protein with amino acid sequence set forth in SEQ ID NO: 30. In some embodiments, the desired/donor DNA sequence is at least 80%, 90%, or 95% identical to SEQ ID NO: 33.

The disclosure also provides vectors for constructing a humanized animal model or a knock-out model. In some embodiments, the vectors comprise sgRNA sequence, wherein the sgRNA sequence target IL36A gene, and the sgRNA is unique on the target sequence of the gene to be altered, and meets the sequence arrangement rule of 5′-NNN (20)-NGG3′ or 5′-CCN—N (20)-3′; and in some embodiments, the targeting site of the sgRNA in the mouse IL36A gene is located on the exon 1, exon 2, exon 3, exon 4, exon 5, exon 6, intron 1, intron 2, intron 3, intron 4, intron 5, upstream of exon 1, or downstream of exon 6 of the mouse IL36A gene.

In some embodiments, the sgRNA targeting sequences are selected from SEQ ID NOs: 40-49. Thus, the disclosure provides sgRNA sequences for constructing a genetic modified animal model. In some embodiments, the oligonucleotide sgRNA sequences comprise any one of SEQ ID NOs: 50-57.

In some embodiments, the disclosure relates to a plasmid construct (e.g., pT7-sgRNA) including the sgRNA sequence, and/or a cell including the construct.

The targeting vector can further include one or more selectable markers, e.g., positive or negative selectable markers. In some embodiments, the positive selectable marker is a Neo gene or Neo cassette. In some embodiments, the negative selectable marker is a DTA gene.

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell. In some embodiments, the cell is an embryonic stem cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous IL1RL2 gene locus, a sequence encoding a region of an endogenous IL1RL2 with a sequence encoding a corresponding region of human or chimeric IL1RL2. In some embodiments, the replacement occurs in a germ cell, a somatic cell, a blastocyst, or a fibroblast, etc. The nucleus of a somatic cell or the fibroblast can be inserted into an enucleated oocyte.

FIGS. 10-12 show a humanization strategy for a mouse IL36A locus. In some embodiments, the targeting strategy involves a vector comprising the 5′ end homologous arm, human IL36A gene fragment, 3′ homologous arm. The process can involve replacing endogenous IL36A sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous IL36A sequence with human IL36A sequence.

Thus, in some embodiments, the methods for making a genetically modified, humanized animal, can include the step of replacing at an endogenous IL36A locus (or site), a nucleic acid encoding a region of endogenous IL36A with a sequence encoding a corresponding region of human IL36A. The sequence can include a region (e.g., a part or the entire region) of exon 1, exon 2, exon 3, and/or exon 4 of a human IL36A gene. In some embodiments, the sequence includes a portion of exon 1, and exons 2-4 of a human IL36A gene (e.g., nucleic acids 414-890 of NM_014440.3). In some embodiments, the region includes the entire coding sequence (CDS) of human IL36A (e.g., SEQ ID NO: 30). In some embodiments, the endogenous IL36A locus is exon 1, exon 2, exon 3, exon 4, exon 5, and/or exon 6 of mouse IL36A. In some embodiments, the sequence includes a portion of exon 2, exons 3-4, and a portion of exon 5 of mouse IL36A gene (e.g., nucleic acids 162-641 of NM_019450.3).

In some embodiments, the methods of modifying a IL36A locus of a mouse to express a chimeric human/mouse IL36A peptide can include the steps of replacing at the endogenous mouse IL36A locus a nucleotide sequence encoding a mouse IL36A with a nucleotide sequence encoding a human IL36A, thereby generating a sequence encoding a humanized IL36A.

In some embodiments, the nucleotide sequence encoding the humanized IL36A can include a first nucleotide sequence including the 5′ UTR of mouse IL36A gene; a second nucleotide sequence including the entire coding sequence of human IL36A gene; and/or a third nucleotide sequence including the 3′ UTR of mouse IL36A gene.

In some embodiments, the disclosure provides inserting in at least one cell of the animal, at an endogenous IL36A gene locus, a sequence encoding the extracellular and transmembrane regions of human IL36A, and the cytoplasmic region of endogenous IL36A. In some embodiments, the insertion occurs in a germ cell, a somatic cell, a blastocyst, or a fibroblast, etc. The nucleus of a somatic cell or the fibroblast can be inserted into an enucleated oocyte.

FIGS. 2-3 show humanization strategies for a mouse IL1RL2 locus. The targeting strategies involve a vector comprising the 5′ end homologous arm, a humanized IL1RL2 sequence, 3′ homologous arm. The process can involve inserting the humanized IL1RL2 sequence by homologous recombination. In some embodiments, the cleavage at the upstream and the downstream of the target site (e.g., by zinc finger nucleases, TALEN or CRISPR) can result in DNA double strands break, and the homologous recombination is used to insert the humanized IL1RL2 sequence within the endogenous IL1RL2 gene locus.

Thus, in some embodiments, the methods for making a genetically modified, humanized animal, can include the step of inserting at an endogenous IL1RL2 locus (or site), a nucleic acid encoding the extracellular and transmembrane regions of human IL1RL2, and the cytoplasmic region of endogenous IL1RL2. The sequence can include a portion of human exon 2, human exons 3-8, a portion of human exon 9, a portion of endogenous exon 9, and endogenous exon 10 and a portion of endogenous exon 11. In some embodiments, the sequence includes nucleic acids 77-1144 of NM_003854.4 and nucleic acids 1315-1959 of NM_133193.4.

In some embodiments, the methods of modifying a IL1RL2 locus of a mouse to express a humanized IL1RL2 peptide can include the steps of inserting at the endogenous mouse IL1RL2 locus a nucleotide sequence encoding a humanized IL1RL2 protein, thereby generating a sequence encoding a humanized IL1RL2.

In some embodiments, the nucleotide sequence encoding the humanized IL1RL2 can include: a first nucleotide sequence encoding the N-terminal 1-5 (e.g., 1, 2, 3, 4, or 5) amino acids of mouse IL1RL2 protein; a second nucleotide sequence encoding the extracellular and transmembrane regions of human IL1RL2 protein; and a third nucleotide sequence encoding the cytoplasmic region of mouse IL1RL2 protein. In some embodiments, the second nucleotide sequence does not include the N-terminal 1-5 (e.g., 1, 2, 3, 4, or 5) amino acids of the extracellular region (including the signal peptide) of human IL1RL2 protein. In some embodiments, the second nucleotide further includes a sequence encoding the N-terminal 1-6 (e.g., 1, 2, 3, 4, 5, or 6) amino acids of the cytoplasmic region of human IL1RL2 protein, and the third nucleotide does not include a corresponding sequence encoding the N-terminal 1-6 (e.g., 1, 2, 3, 4, 5, or 6) amino acids of the cytoplasmic region of mouse IL1RL2 protein. In some embodiments, the first nucleotide sequence is optional.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing IL36A and/or IL1RL2 gene humanized animal models, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudopregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

In some embodiments, methods of making the genetically modified animal comprises modifying the coding frame of the non-human animal's IL36A and/or IL1RL2 genes, e.g., by inserting a nucleotide sequence (e.g., DNA or cDNA sequence) encoding human or humanized IL36A and/or IL1RL2 proteins immediately after the endogenous regulatory element of the non-human animal's IL36A and/or IL1RL2 genes. For example, one or more functional region sequences of the non-human animal's IL36A and/or IL1RL2 genes can be knocked out, or inserted with a sequence, such that the non-human animal cannot express or expresses a decreased level of endogenous IL36A and/or IL1RL2 proteins. In some embodiments, the coding frame of the modified non-human animal's IL36A gene can be all or part of the nucleotide sequence from exon 1 to exon 6 of the non-human animal's IL36A gene. In some embodiments, the coding frame of the modified non-human animal's IL1RL2 gene can be all or part of the nucleotide sequence from exon 1 to exon 11 of the non-human animal's IL1RL2 gene.

In some embodiments, methods of making the genetically modified animal comprises inserting a nucleotide sequence encoding human or humanized IL36A and/or IL1RL2 proteins and/or an auxiliary sequence after the endogenous regulatory element of the non-human animal's IL36A and/or IL1RL2 genes. In some embodiments, the auxiliary sequence can be a stop codon, such that the IL36A and/or IL1RL2 gene humanized animal models can express human or humanized IL36A and/or IL1RL2 proteins in vivo, but does not express non-human animal's IL36A and/or IL1RL2 proteins. In some embodiments, the auxiliary sequence includes WPRE (WHP Posttranscriptional Response Element), lox2, and/or polyA.

In some embodiments, the method for making the genetically modified animal comprises:
(1) providing a plasmid comprising a human IL36A/IL1RL2 gene fragment, flanked by a 5′ homology arm and a 3′ homology arm, wherein the 5′ and 3′ homology arms target an endogenous IL36A/IL1RL2 gene;
(2) providing one or more small guide RNAs (sgRNAs) that target the endogenous IL36A/IL1RL2 gene;
(3) modifying genome of a fertilized egg or an embryonic stem cell by using the plasmid of step (1), the sgRNAs of step (2), and Cas9;
(4) transplanting the fertilized egg obtained in step (3) into the oviduct of a pseudopregnant female mouse or transplanting the embryonic stem cell obtained in step (3) into a blastocyst which is then transplanted into the oviduct of a pseudopregnant female mouse to produce a child mouse that functionally expresses a humanized IL36A/IL1RL2 protein; and
(5) mating the child mouse obtained in step (2) to obtain a homozygote mouse.

In some embodiments, the fertilized egg is modified by CRISPR with sgRNAs that target a 5′-terminal targeting site and a 3′-terminal targeting site.

In some embodiments, the sequence encoding the humanized IL36A/IL1RL2 protein is operably linked to an endogenous regulatory element at the endogenous IL36A/IL1RL2 gene locus.

In some embodiments, the genetically-modified animal does not express an endogenous IL36A/IL1RL2 protein.

In some embodiments, the method for making the genetically modified animal comprises:
(1) providing a plasmid comprising a human or chimeric IL36A/IL1RL2 gene fragment, flanked by a 5′ homology arm and a 3′ homology arm, wherein the 5′ and 3′ homology arms target an endogenous IL1RL2 gene;
(2) providing one small guide RNAs (sgRNAs) that target the endogenous IL36A/IL1RL2 gene; and
(3) modifying genome of a fertilized egg or an embryonic stem cell by inserting the human or chimeric IL36A/IL1RL2 gene fragment into the genome.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement or insertion with human sequence at an endogenous locus under control of endogenous regulatory elements provides a physiologically appropriate expression pattern and level that results in a useful humanized animal whose physiology with respect to the replaced or disrupted gene are meaningful and appropriate in the context of the humanized animal's physiology.

Genetically modified animals that express human or humanized IL36A and/or IL1RL2 proteins, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized IL36A and/or IL1RL2, which are useful for testing agents that can decrease or block the interaction between the interaction between IL36A (or variant thereof) and its receptor (e.g., IL1RL2), the interaction between IL36A and anti-human IL36A antibodies, and the interaction between IL1RL2 and anti-IL1RL2 antibodies, testing whether an agent can increase or decrease the immune response, and/or determining whether an agent is an IL36A/IL1RL2 agonist or antagonist. The genetically modified animals can be, e.g., an animal model of a human disease, e.g., the disease is induced genetically (a knock-in or knockout). In various embodiments, the genetically modified non-human animals further comprise an immune system disorder (e.g., psoriasis). In some embodiments, the anti-IL36A antibody or anti-IL1RL2 antibody blocks or inhibits the IL36A/IL1RL2-mediated signaling pathway.

In some embodiments, the anti-IL36A antibody described herein can block the interaction between IL36A and IL1RL2, thereby inhibiting IL36A/IL1RL2 signaling. In some embodiments, the anti-IL1RL2 antibody described herein can block the interaction between IL1RL2 and IL36A, thereby inhibiting IL36A/IL1RL2 signaling.

In some embodiments, the genetically modified animals can be used for determining effectiveness of a therapeutic agent (e.g., an anti-IL36A antibody or an anti-IL1RL2 antibody) for the treatment of various immune disorders, including psoriasis. Thus, the methods as described herein can be used to determine the effectiveness of an anti-IL36A or anti-IL1RL2 antibody in inhibiting immune response. In some embodiments, the immune disorders described herein is psoriasis, allergy, asthma, myocarditis, nephritis, hepatitis, systemic lupus erythematosus, rheumatoid arthritis, scleroderma, hyperthyroidism, idiopathic thrombocytopeni purpura, autoimmune hemolytic anemia, ulcerative colitis, autoimmune liver disease, diabetes, pain or neurological disorders.

In some embodiments, the immune disorder described herein is psoriasis, and the animal model is established by inducing the animal (e.g., any of the animals described herein) with imiquimod (IMQ). In some embodiments, the method involves administering the therapeutic agent (e.g., an anti-IL36A antibody or an anti-IL1RL2 antibody) to the animal as described herein (e.g., by intraperitoneal injection), wherein the animal has IMQ induced psoriasis; and determining effects of the therapeutic agent in treating psoriasis. In some embodiments, the effects are evaluated by comparing skin damage parameters (e.g., rash and sesquamation) of the animal with an animal induced by IMQ, but not treated with the therapeutic agent. For example, reduced rash and sesquamation indicate that the therapeutic agent can inhibit immune response thereby treating psoriasis.

In some embodiments, the immune disorder described herein is asthma, and the animal model is established by inducing the animal (e.g., any of the animals described herein) with ovalbumin (OVA) and aluminum hydroxide. In some embodiments, the method involves administering the therapeutic agent (e.g., an anti-IL36A antibody or an anti-IL1RL2 antibody) to the animal as described herein (e.g., by intraperitoneal injection), wherein the animal has asthma; and determining effects of the therapeutic agent in treating asthma. In some embodiments, the effects are evaluated by comparing serum IgE level; pathological lung histology features; number of inflammatory cells (e.g., eosinophil counts in infiltrating cells) in bronchoalveolar lavage fluid (BALF); and/or airway reactivity of the animal with an animal induced by OVA/aluminum hydroxide, but not treated with the therapeutic agent. For example, reduced serum IgE level and/or reduced number of inflammatory cells in BALF indicate that the therapeutic agent can inhibit immune response thereby treating asthma.

In some embodiments, the immune disorder described herein is atopic dermatitis, and the animal model is established by inducing the animal (e.g., any of the animals described herein) with oxazolone (OXA), e.g., by smearing 0.1%-1% OXA to an exposed skin of the animal (e.g., ears or back). In some embodiments, the animal's skin is smeared with about 0.1%, about 0.2%, about 0.3%, about 0.4%, about 0.5%, about 0.6%, about 0.7%, about 0.8%, about 0.9 or about 1% OXA for about 7-11 days, about 7-18 days, or about 7-26 days. In some embodiments, the method involves administering the therapeutic agent (e.g., an anti-IL36A antibody or an anti-IL1RL2 antibody) to the animal as described herein (e.g., by intraperitoneal injection), wherein the animal has atopic dermatitis; and determining effects of the therapeutic agent in treating atopic dermatitis. In some embodiments, the effects are evaluated by comparing epidermal stromal cell hyperplasia; erosion/scab; hyperkeratosis; dermal and subcutaneous mixed inflammatory cell infiltration; eosinophilic infiltration; serum IgE levels; and/or ear thickness of the animal with an animal that induced by OXA, but not treated with the therapeutic agent. For example, reduced ear thickness, reduced serum IgE level, and/or reduced eosinophil infiltration indicate that the therapeutic agent can inhibit immune response thereby treating atopic dermatitis.

In some embodiments, the genetically modified animals can be used for determining effectiveness of a therapeutic agent (e.g., an anti-IL36A antibody or an anti-IL1RL2 antibody) for the treatment of various autoimmune diseases, including inflammatory arthritis, eczema, eosinophilic esophagitis, rheumatoid arthritis, Crohn's disease, systemic lupus erythematosus, ankylosing spondylitis, inflammatory bowel diseases (IBD), ulcerative colitis, multiple sclerosis, systemic juvenile idiopathic arthritis (SJIA), and scleroderma.

In some embodiments, the genetically modified animals can be used for determining effectiveness of a therapeutic agent (e.g., an anti-IL36A antibody or an anti-IL1RL2 antibody) for the reducing an inflammation or infection (e.g., Staphylococcus aureus infection, helminth infection, or viral infection). In some embodiments, In some embodiments, the genetically modified animals can be used for determining effectiveness of a therapeutic agent (e.g., an anti-IL36A antibody or an anti-IL1RL2 antibody) for treating chronic inflammatory diseases (e.g., chronic obstructive pulmonary disease, idiopathic pulmonary fibrosis, rheumatoid arthritis, or ulcerative colitis).

In some embodiments, the genetically modified animals can be used for determining effectiveness of a therapeutic agent (e.g., an anti-IL36A antibody or an anti-IL1RL2 antibody) for the treatment of cancer. In some embodiments, the methods involve administering the therapeutic agent (e.g., an anti-IL36A antibody or an anti-IL1RL2 antibody) to the animal as described herein, wherein the animal has a cancer or tumor; and determining inhibitory effects of the therapeutic agent to the cancer or tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT. In addition, a delicate balance is required for these antibodies, as IL36A and IL1RL2 are also expressed on many other cells. Thus, it is important that the humanized IL36A and/or IL1RL2 functions in a largely similar way as compared to the endogenous IL36A and/or IL1RL2, so that the results in the humanized animals can be used to predict the efficacy or toxicity of these therapeutic agents in the human.

In some embodiments, the tumor comprises one or more cancer cells (e.g., human or mouse cancer cells) that are injected into the animal. In some embodiments, the therapeutic agent inhibits IL36A/IL1RL2 signaling pathways. In some embodiments, the therapeutic agent does not inhibit IL36A/IL1RL2 signaling pathways.

In some embodiments, the genetically modified animals can be used for determining whether an anti-IL36A or anti-IL1RL2 antibody is an agonist or antagonist. In some embodiments, the methods as described herein are also designed to determine the functional effects of the therapeutic agent (e.g., an anti-IL36A antibody or an anti-IL1RL2 antibody), e.g., whether the agent can upregulate the immune response or downregulate immune response, and/or whether the agent can induce complement mediated cytotoxicity (CMC) or antibody dependent cellular cytotoxicity (ADCC). In some embodiments, the genetically modified animals can be used for determining the effective dosage of a therapeutic agent for treating a disease in the subject, e.g., immune disorders.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the therapeutic agent (e.g., an anti-IL36A antibody or an anti-IL1RL2 antibody) is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genitourinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer described herein is lymphoma, non-small cell lung cancer, cervical cancer, leukemia, ovarian cancer, nasopharyngeal cancer, breast cancer, endometrial cancer, colon cancer, rectal cancer, gastric cancer, bladder cancer, glioma, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myeloproliferation abnormal syndromes, and sarcomas. In some embodiments, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin's lymphoma and non-Hodgkin's lymphoma, including B-cell lymphoma, diffuse large B-cell lymphoma, follicular lymphoma, mantle cell lymphoma, marginal zone B-cell lymphoma, T-cell lymphoma, and Waldenstrom macroglobulinemia. In some embodiments, the sarcoma is selected from the group consisting of osteosarcoma, Ewing sarcoma, leiomyosarcoma, synovial sarcoma, soft tissue sarcoma, angiosarcoma, liposarcoma, fibrosarcoma, rhabdomyosarcoma, and chondrosarcoma. In a specific embodiment, the tumor is breast cancer, ovarian cancer, endometrial cancer, melanoma, kidney cancer, lung cancer, or liver cancer. In some embodiments, the cancer described herein is acute lymphocytic leukaemia or a solid tumor.

The present disclosure also provides methods of determining toxicity of a therapeutic agent (e.g., an anti-IL36A antibody or an anti-IL1RL2 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the IL36A and/or IL1RL2 gene functions, human IL36A and/or IL1RL2 antibodies, drugs or efficacies for human IL36A and/or IL1RL2 targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the IL36A and/or IL1RL2 gene humanized non-human animal prepared by the methods described herein, the IL36A and/or IL1RL2 gene humanized non-human animal described herein, the double- or multi-humanized non-human animal generated by the methods described herein (or progeny thereof), a non-human animal expressing the human or humanized IL36A and/or IL1RL2 proteins, or the tumor-bearing or inflammatory animal models described herein. In some embodiments, the TCR-T, CAR-T, and/or other immunotherapies can treat the IL36A/IL1RL2-associated diseases described herein. In some embodiments, the TCA-T, CAR-T, and/or other immunotherapies provides an evaluation method for treating the IL36A/IL1RL2-associated diseases described herein.

Genetically Modified Animal Model with Two or More Human or Chimeric Genes

The present disclosure further relates to methods for generating genetically modified animal model with two or more human or chimeric genes. The animal can comprise a human or chimeric IL36A and/or IL1RL2 genes and a sequence encoding an additional human or chimeric protein.

In some embodiments, the additional human or chimeric protein can be interleukin 33 (IL33), interleukin 7 receptor (IL7R), interleukin 6 (IL6), interleukin 12 (IL12), interleukin 23 (IL23), Tumor Necrosis Factor alpha (TNF-α), interleukin 4 (IL4), programmed cell death protein 1 (PD1), Tumor Necrosis Factor Receptor Superfamily, Member 7 (CD27), tumor necrosis factor receptor superfamily member 9 (4-1BB), cytotoxic T-lymphocyte-associated protein 4 (CTLA4), prostate-specific membrane antigen (PSMA), Tumor necrosis factor receptor superfamily, member 4 (OX40), T cell immunoreceptor with Ig and ITIM domains (TIGIT), and/or lymphocyte-activation gene 3 (LAG3).

The methods of generating genetically modified animal model with two or more human or chimeric genes (e.g., humanized genes) can include the following steps:
(a) using the methods of introducing human IL36A and/or IL1RL2 genes or chimeric IL36A and/or IL1RL2 genes as described herein to obtain a genetically modified non-human animal;
(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric, IL33, IL7R, IL6, IL12, IL23, TNF-α, IL4, CD47, PD1, CD27, 4-1BB, CTLA4, PSMA, OX40, TIGIT, and/or LAG3. Some of these genetically modified non-human animals are described, e.g., in PCT/CN2020/105529, PCT/CN2017/090320, PCT/CN2017/099577, PCT/CN2017/099575, PCT/CN2017/099576, PCT/CN2017/099574, PCT/CN2017/110435, PCT/CN2017/120388, PCT/CN2018/081628, PCT/CN2019/110819, PCT/CN/2019/126045; each of which is incorporated herein by reference in its entirety.

In some embodiments, the IL36A and/or IL1RL2 humanizations are directly performed on a genetically modified animal having a human or chimeric IL33, IL7R, IL6, IL12, IL23, TNF-α, IL4, CD47, PD1, CD27, 4-1BB, CTLA4, PSMA, OX40, TIGIT, and/or LAG3 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-IL36A antibody or an anti-IL1RL2 antibody, and an additional therapeutic agent for the treatment of cancer or immune disorder (e.g., psoriasis). The methods include administering the an anti-IL36A antibody or an anti-IL1RL2 antibody and the additional therapeutic agent to the animal, wherein the animal has a tumor or immune disorder; and determining effects of the combined treatment to the tumor or immune disorder. In some embodiments, the additional therapeutic agent is an antibody that specifically binds to, IL33, IL7R, IL6, IL12, IL23, TNF-α, IL4, CD47, PD1, CD27, 4-1BB, CTLA4, PSMA, OX40, TIGIT, and/or LAG3. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancers as described herein, e.g., bladder cancer, melanoma, leukemia, hepatocellular carcinoma, breast cancer, sarcoma, head and neck cancer, colon cancer, lung cancer, liver cancer, or brain cancer.

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

The following materials were used in the following examples.

C57BL/6 mice and Flp transgenic mice were purchased from the China Food and Drugs Research Institute National Rodent Experimental Animal Center.

Lipopolysaccharides from Escherichia coli O111: B4 were purchased from Sigma, catalog number: L2630;

Attune Nxt Acoustic Focusing Cytometer was purchased from Thermo Fisher, model Attune Nxt;

PrimeScript 1st Strand cDNA Synthesis Kit was purchased from TAKARA, model 6110A;

Heraeus™ Fresco™ 21 Microcentrifuge was purchased from Thermo Fisher, model Fresco 21;

SpelI, EcoRV, StuI, BglII, AseI, BbsI enzymes were purchased from NEB, and the product numbers are R0133M, R0195M, R0187M, R0144M, R0526, R0539L;

UCA kit was obtained from Biocytogen Pharmaceuticals (Beijing) Co., Ltd., product number BCG-DX-001;

Ambion In Vitro Transcription Kit was purchased from Ambion, Cat. No. AM1354;

Cas9mRNA was purchased from SIGMA, Cat. No. CAS9MRNA-1EA;

Recombinant Anti-IL36 alpha/IL-1F6 antibody [EPR23152-241] was purchased from Abcam, Cat. No. ab269274;

Recombinant Anti-IL36 alpha/IL-1F6 antibody [EPR23089-87] was purchased from Abcam, Cat. No. ab269271;

GAPDH Mouse Monoclonal Antibody was purchased from Biyuntian, Cat. No. AF5009;

Anti-IL-36R antibody was purchased from Abcam, catalog number ab180894;

Purified anti-mouse CD16/32 Antibody was purchased from Biolegend, Cat. No. 101302;

Zombie NIR™ Fixable Viability Kit from Biolegend, Cat. No. 423106;

Brilliant Violet 510™ anti-mouse CD45 Antibody was purchased from Biolegend, Cat. No. 103138;

PerCP anti-mouse Ly-6G/Ly-6C (Gr-1) Antibody was purchased from Biolegend, Cat. No. 108426;

Brilliant Violet 421™ anti-mouse CD4 Antibody was purchased from Biolegend, Cat. No. 100438;

FITC anti-mouse F4/80 Antibody was purchased from Biolegend, Cat. No. 123108;

PE anti-mouse CD8a Antibody was purchased from Biolegend, Cat. No. 100708;

PE/Cy™ 7 Mouse anti-mouse NK1.1 Antibody was purchased from BD Pharmingen, Cat. No. 552878;

APC anti-mouse/rat Foxp3 Antibody was purchased from eBioscience, Cat. No. 17-5773-82;

FITC anti-Mouse CD19 Antibody was purchased from Biolegend, Cat. No. 115506;

PerCP/Cy5.5 anti-mouse TCR β chain was purchased from Biolegend, Cat. No. 109228;

Brilliant Violet 605™ anti-mouse CD11c Antibody was purchased from Biolegend, Cat. No. 117334;

PE anti-mouse/human CD11b Antibody was purchased from Biolegend, Cat. No. 101208.

### Example 1 Preparation of IL1RL2 Gene Humanized Mice

Mouse IL1RL2 gene (NCBI Gene ID: 107527, Primary source: MGI: 1913107, UniProt ID: Q9ERS7, located at positions 40324589 to 40367555 of chromosome 1 (NC_000067.6), based on transcript NM_133193.4 and its encoded protein NP_573456.1 (SEQ ID NO: 1)) and human IL1RL2 gene (NCBI Gene ID: 8808, Primary source: HGNC: 5999, UniProt ID: Q9HB29, located at positions 102186973 to 102242910 of chromosome 2 (NC_000002.12), based on transcript NM_003854. 4 and its encoded protein NP_003845.2 (SEQ ID NO: 2)) are shown in FIG. 1.

The human IL1RL2 protein coding sequence can be inserted into the mouse endogenous IL1RL2 locus, or the human IL1RL2 coding sequence can be used to replace the mouse corresponding sequence, so that the mouse expresses a human or humanized IL1RL2 protein. Taking the insertion of the partial coding sequence of human IL1RL2 protein as an example, through gene editing technologies, under the control of mouse endogenous IL1RL2 regulatory elements, the coding sequences of human IL1RL2 protein signal peptide, transmembrane region and extracellular region, and the mouse cytoplasmic region were inserted into mouse exon 2 (at the start codon). Further, the endogenous 3′UTR and polyA were inserted after the mouse cytoplasmic region coding sequence to obtain the mouse humanized IL1RL2 locus. A schematic diagram is shown in FIG. 2.

The targeting strategy is shown in FIG. 3. The targeting vector V1 in the figure contains a 5′ homology arm (SEQ ID NO: 3), a 3′ homology arm (SEQ ID NO: 4) and an A1 fragment that contains human IL1RL2 (hIL1RL2) sequence (SEQ ID NO: 5) and mouse IL1RL2 (mIL1RL2) sequence (SEQ ID NO: 6). The 5′ homology arm contains the nucleotide sequence of positions 40322546-40326494 of NCBI accession number NC_000067.6. The 3′ homology arm contains the nucleotide sequence of positions 40326498-40330768 of NCBI Accession No. NC_000067.6. On the A1 fragment, human IL1RL2 sequence fragment, mouse IL1RL2 sequence fragment, and polyA are sequentially connected from 5′ to 3′. The human IL1RL2 sequence contains the nucleotide sequence of positions 77-1144 of the NCBI accession number NM_003854.4. The mouse IL1RL2 sequence contains the nucleotide sequence of positions 1315-4072 of the NCBI accession number NM_133193.4. The polyA sequence is shown in SEQ ID NO: 69. The connection between the upstream of the human IL1RL2 sequence and the mouse sequence is designed as 5′-

(SEQ ID NO: 7)
TACTTCTACCATTGTCCTCTCTTTTCCTTTGTAGCCTGAGATGTGGTCC
TTGCTGCTCTGCGGGTTGTCCATCGCCCTTC-3′,

where the last “G” of the sequence “CTGAG” is the last nucleotide of mouse sequence and the “A” of sequence “ATGTG” is the first nucleoside of human sequence. The connection downstream of the mouse IL1RL2 sequence to polyA was designed as 5′-

(SEQ ID NO: 8)
CAGTACATTGACATTTTAAATAAAGTATATTTTAATAAAAATCGATACC
GTCGACCTCGACTGTGCCTTCTAGTTGCCAG-3′,

where the last “A” of the sequence “TAAAA” is the last nucleotide of the mouse sequence, and the first “A” of “ATCGA” is the first nucleotide of polyA. The mRNA sequence of the modified humanized mouse IL1RL2 and its encoded protein sequence are shown in SEQ ID NO: 9 and SEQ ID NO: 10, respectively.

The targeting vector V1 also includes a resistance gene for positive clone screening, namely the coding sequence of neomycin phosphotransferase (Neo), and two site-specific recombination sites arranged in the same direction (Frt recombination sites) on both sides of the resistance gene, forming a Neo cassette. The connection between the 5′ end of Neo cassette and polyA is designed as 5′-

(SEQ ID NO: 11)
AGGGGGAGGATTGGGAAGACAATAGCAGGCATGCTGGGGAGATATCGAA
TTCCGAAGTTCCTATTCTCTAGAAAGTATAG-3′,

wherein the “A” of sequence “GGGGA” is the last nucleotide of polyA sequence, and the “G” in the sequence “GATAT” is the first nucleotide of the Neo cassette. The connection between the 3′ end of the Neo cassette and the mouse sequence is designed as 5′-

(SEQ ID NO: 12)
ATAGGAACTTCATCAGTCAGGTACATAATTAGGTGGATCCGGGGTTACA
TCTTTGCTCTTCTGTGGGGTGTTTTTCCTGC-3′,

where the last “C” in the sequence “GATCC” is the last nucleotide of the Neo cassette, and the first “G” of the sequence “GGGGT” is the first nucleotide of the mouse sequence. In addition, a negative selection marker (the coding sequence for the A subunit of diphtheria toxin (DTA)) was also constructed downstream of the 3′ homology arm of the recombinant vector.

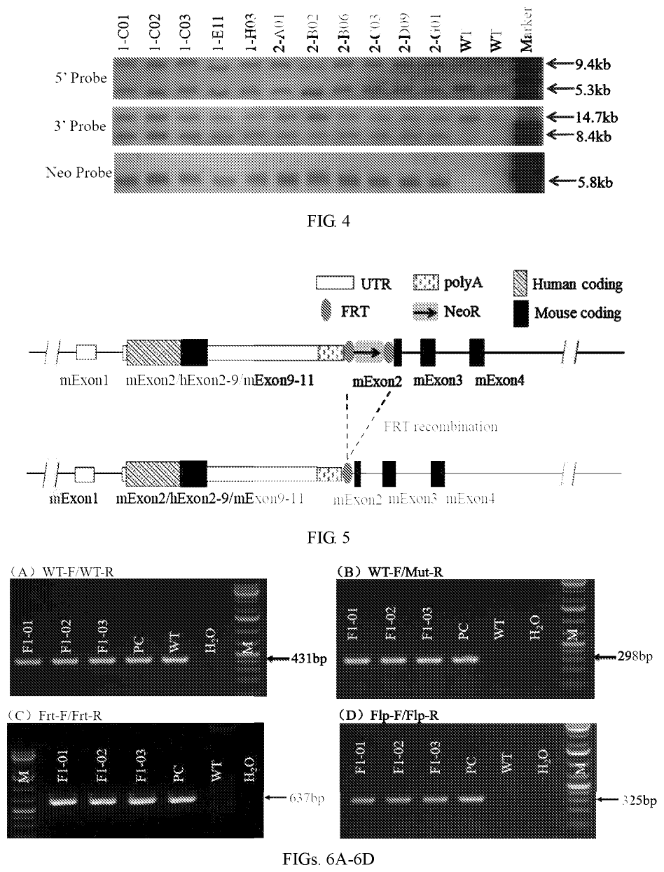

The construction of the targeting vector can be carried out by conventional methods, such as restriction enzyme cleavage/ligation, direct synthesis and the like. The constructed recombinant vector is preliminarily verified by enzyme digestion, and then sent to a sequencing company for sequencing verification. The recombinant vector verified by sequencing was electroporated into embryonic stem cells of C57BL/6 mice, and the obtained cells were screened by positive clone selection marker genes, and the integration of foreign genes was confirmed by PCR and Southern Blot. Specifically, Southern Blot was performed on the clones identified as positive by PCR (the cellular DNA was digested with Spel or EcoRV or StuI, respectively, and 3 probes were used for hybridization. The specific probes and the lengths of the target fragments are shown in the table below). Southern Blot results are shown in FIG. 4. The test results showed that among the 11 clones identified as positive by PCR, and further verified by sequencing, except for 2-B02, the other 10 were positive heterozygous without random insertion. These 10 positive clones were 1-C01, 1-C02, 1-C03, 1-E11, 1-H03, 2-A01, 2-B06, 2-C03, 2-D09, and 2-G01.

TABLE 5
Specific probes and target fragment lengths

| Restriction | | Wildtype fragment | Recombinant |
|---|---|---|---|
| endonuclease | Probe | size | fragment size |
| Spel | 5′Probe | 5.3 kb | 9.4 kb |
| EcoRV | 3′Probe | 14.7 kb | 8.4 kb |
| StuI | Neo Probe | — | 5.8 kb |

The PCR assay includes the following primers:

CL-F1:
(SEQ ID NO: 13)
5′-CTGAGGCTCTGTGGTGCTGGTTATC-3′,
CL-R1:
(SEQ ID NO: 14)
5′-TGTCCTTGCATCCATCTGCTGTGACAG-3′;
CL-F2:
(SEQ ID NO: 15)
5′-GCTCGACTAGAGCTTGCGGA-3′,
CL-R2:
(SEQ ID NO: 16)
5′-GTCAAGCAAGATGCGGCTTAGAAGG-3′

Southern Blot detection includes the following probe primers:

5′ Probe:
5′Probe-F:
(SEQ ID NO: 17)
5′-CTAACCATCTTCTTTCTGCAGATGAC-3′,
5′Probe-R:
(SEQ ID NO: 18)
5′-GCTAGTTTTCCAGACGGCTGTCCAT-3′;
3′ Probe:
3′Probe-F:
(SEQ ID NO: 19)
5′-CTGGTTCTCCTAATCCCTTTGTTTG-3′,
3′Probe-R:
(SEQ ID NO: 20)
5′-CTTTCAGTATTTACATTCTCTTCAATACAC-3′;
Neo Probe:
Neo Probe-F:
(SEQ ID NO: 21)
5′-GGATCGGCCATTGAACAAGAT-3′,
Neo Probe-R:
(SEQ ID NO: 22)
5′-CAGAAGAACTCGTCAAGAAGGC-3′.

The screened correct positive cloned cells (black mice) were introduced into the isolated blastocysts (white mice), and the obtained chimeric blastocysts were placed in a cell culture medium for a short-term culture and then transplanted to the fallopian tubes of female recipient mice (white mice) to produce F0 generation chimeric mice (black and white). The F0 generation chimeric mice were backcrossed with the wild-type mice to obtain the F1 generation mice. Next, the F1 generation heterozygous mice were bred to each other to obtain the F2 generation homozygous mice. The positive cloned selection marker gene can also be removed by mating the positive mice with the Flp tool mice (see FIG. 5 for the schematic diagram of the process). The IL1RL2 humanized homozygous mouse can then be obtained by mating with each other. The genotype of the offspring mouse somatic cells can be identified by PCR (primers are shown in the table below), and the identification results of an exemplary F1 generation mouse (the Neo marker gene has been removed) are shown in FIGS. 6A-6D, where F1-01, F1-02 and F1-03 were all positive heterozygous mice. The results showed that IL1RL2 humanized genes that can be stably passed on to offspring without random insertion.

TABLE 6
Primer names and sequences

| Primer | SEQ ID NO | Sequence (5′-3′) | Fragment size |
|---|---|---|---|
| WT-F | SEQ ID NO: 23 | GGCGTTTACTGGCTTCTGAAGGTCT | WT: 431 bp |
| WT-R | SEQ ID NO: 24 | AAAGAGTGACTAGCAAGGTTGGGGC | Mut: 6352 bp |
| WT-F | SEQ ID NO: 23 | GGCGTTTACTGGCTTCTGAAGGTCT | Mut: 298 bp |
| Mut-R | SEQ ID NO: 14 | TGTCCTTGCATCCATCTGCTGTGACAG | |
| Frt-F | SEQ ID NO: 25 | AGCTTTCCTCTTGCCAACATGTCTT | Mut: 2431 bp |
| Frt-R | SEQ ID NO: 26 | AAAGAGTGACTAGCAAGGTTGGGGC | Mut: 637 bp |
| | | | (after Neo |
| | | | removal) |
| Flp-F | SEQ ID NO: 27 | GACAAGCGTTAGTAGGCACATATAC | Mut: 325 bp |
| Flp-R | SEQ ID NO: 28 | GCTCCAATTTCCCACAACATTAGT | (after Neo |
| | | | removal) |

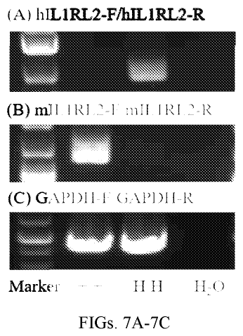

The expression of humanized IL1RL2 mRNA in mice can be confirmed by conventional detection methods, such as RT-PCR. Specifically, 8-week-old male C57BL/6 wild-type mice and one IL1RL2 gene humanized homozygous mouse prepared in this example were selected. After euthanasia, lung tissues were collected and ground to prepare cell suspensions. After extracting the cellular RNA according to the TRIzol kit instructions, the cellular RNA was reverse transcribed into cDNA. The primers shown in the table below were used for RT-PCR detection, and the results are shown in FIGS. 7A-7C. As can be seen from the figure, only murine IL1RL2 mRNA was detected in C57BL/6 wild-type mice (+/+) (FIG. 7B); and only humanized IL1RL2 mRNA was detected in mice homozygous for humanized IL1RL2 gene (H/H) (FIG. 7A).

TABLE 7
RT-PCR primer sequences and target fragment sizes

| | | Target |
|---|---|---|
| Primer and SEQ ID NO | Primer sequence (5′-3′) | fragment size |
| hIL1RL2-F (SEQ ID NO: 70) | GGACTGTAACGAGATTAAAGGGGAG | Mut: 910 bp |
| hIL1RL2-R (SEQ ID NO: 71) | TGACAAGTTCTTTAGAAAGCCGAAG | |
| mIL1RL2-F (SEQ ID NO: 72) | GTGAACACCAAGGAAGTTGAGTATG | WT: 617 bp |
| mIL1RL2-R (SEQ ID NO:73) | TTATATCCACACTGTTTCTCCAGCA | |
| GAPDH-F (SEQ ID NO: 74) | TCACCATCTTCCAGGAGCGAGA | WT: 479 bp |
| GAPDH-R (SEQ ID NO: 75) | GAAGGCCATGCCAGTGAGCTT | |

Further, 9-week-old female C57BL/6 wild-type mice and IL1RL2 gene humanized homozygous mice were used to model psoriasis using imiquimod (IMQ). IHC staining was used to detect human IL1RL2 protein expression in vivo in the mice. Specifically, four 9-week-old C57BL/6 wild-type mice and two IL1RL2 gene humanized homozygous mice prepared in this example were selected and divided into a control group (G1) and two modeling groups (G2, G3). The back hair of the mice was removed with a shaver 3 days before the experiment, exposing a skin area of 2 cm×4 cm. After 3 days (DO), 10 mg/cm² IMQ cream was used to smear the back skin area of the modeling group mice every day for 5 consecutive days, while Vaseline was used for the control group mice. The specific grouping and modeling methods are shown in the table below.

TABLE 8
Grouping of mice and modeling method

| Group | | Mice | Treatment |
|---|---|---|---|
| Control | G1 | C57BL/6(n = 2) | Vaseline |
| Modeling | G2 | C57BL/6(n = 2) | IMQ |
| | G3 | IL1RL2 humanized mice | IMQ |

The mice in each group were euthanized on the 6th day after modeling. The dorsal skin, small intestine, liver, and brain tissues of mice were taken for OCT-embedded frozen sections, and the human-mouse IL1RL2 protein cross-recognizing antibody Anti-IL-36R (ab180894) was used to detect the expression of humanized IL1RL2 protein in each tissue. The staining results are shown in FIG. 8, and the IHC scoring results are shown in the table below. Scoring items included staining intensity and percentage of positive cells. The staining intensity score was as follows: colorless 0 points, pale yellow 1 point, brownish yellow 2 points, tan 3 points. The percentage of positive cells was scored as 0 points for negative cells, 1 point for ≤10%, and 2 point for 11%-50%, 3 points for 51%-75%, 4 points for >75%. The total score is the score of staining intensity multiplied by the score of percentage of positive cells. A total score of 0 means no positive staining. A total score of 1-3 means suspiciously positive. A total score of 4-6 means weakly positive. A total score of 7-9 means moderately positive. A total score of 10-12 means strongly positive.

TABLE 9
Total score of IHC score in each tissue of mice

| | small intestine | Brain | Skin | liver |
|---|---|---|---|---|
| G1 | 0 | 0 | 0 | 0 |
| | 0 | 0 | 0 | 0 |
| G2 | 4.5 | 0 | 3 | 0 |
| | 0 | 0 | 2 | 0 |
| G3 | 4.5 | 0 | 3 | 0 |
| | 6 | 0 | 3 | 3 |

In the G1 group, the results of IHC staining showed that all tissues of the 2 mice were negative, and the expression of IL1RL2 protein was not detected. In the G2 group, IL1RL2 protein was detected in the small intestine and skin tissue of the mice. In the G3 group, IL1RL2 protein was detected in both the small intestine and skin tissue of both two mice, and the expression of IL1RL2 protein was also detected in liver tissue of one mouse. Since the detection antibody Anti-IL-36R antibody (ab180894) can cross-recognize human and mouse IL1RL2 protein, combined with the RT-PCR detection results in FIGS. 7A-7C, only mouse IL1RL2 protein was detected in G2, and only humanized IL1RL2 protein was detected in G3. The results showed that the IL1RL2 humanized mice prepared by this method can successfully express the humanized IL1RL2 protein.

Further, the in vivo immunophenotyping of IL1RL2 humanized homozygous mice was determined by flow cytometry. Specifically, 7-week-old female C57BL/6 wild-type mice (+/+) and three IL1RL2 humanized homozygous mice (H/H) prepared in this example were selected, and euthanized by de-cervical euthanasia. The spleen, lymph nodes and blood tissues were collected and stained with anti-mouse CD16/32 antibody, Brilliant Violet 510™ anti-mouse CD45 antibody, PerCP anti-mouse Ly-6G/Ly-6C (Gr-1) antibody, Brilliant Violet 421™ anti-mouse CD4 antibody, FITC anti-mouse F4/80 antibody, PE anti-mouse CD8a antibody, PE/Cy™ 7 mouse anti-mouse NK1.1 antibody, APC anti-mouse/rat Foxp3 antibody, FITC anti-mouse CD19 antibody, PerCP/Cy5.5 anti-mouse TCR β chain, APC hamster anti-mouse TCR β Chain, Brilliant Violet 605™ anti-mouse CD11c antibody or PE anti-mouse/human CD11b antibody for flow cytometry analysis.

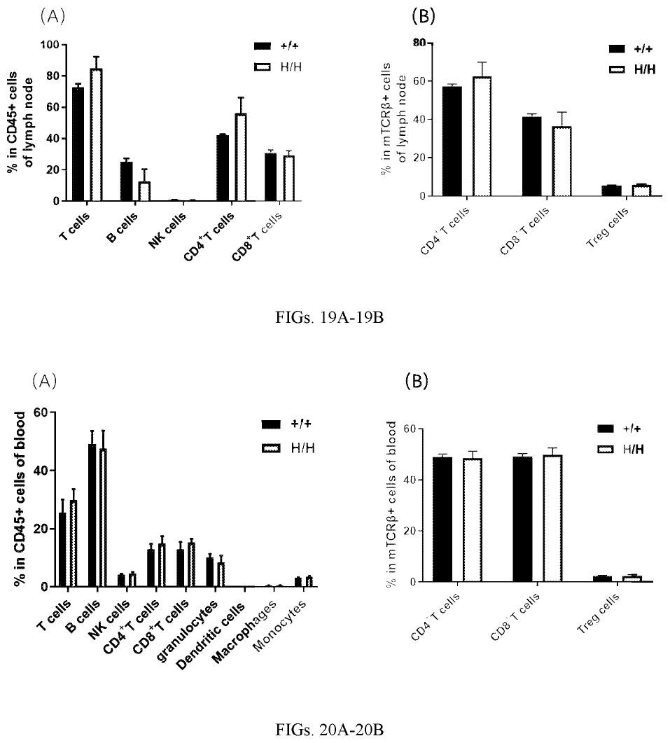

The detection results of lymphocyte (characterized as CD45+) subtype and T cell subtype in spleen, lymph nodes and blood are shown in FIGS. 18A-18B, FIGS. 19A-19B and FIGS. 20A-20B, respectively. The data showed that the percentages of lymphocyte subtypes in the spleen, lymph nodes and blood of IL1RL2 humanized mice, including T cells (T cells), B cells (B cells), NK cells (NK cells), CD4+ T cells (CD4+ cells) T cells), CD8+ T cells (CD8+ T cells), granulocytes (granulocytes), dendritic cells (Dendritic cells), macrophages (Macrophages), and monocytes (Monocytes) percentages are basically the same as the percentages of lymphocyte subtypes in C57BL/6 wild type mice. The percentages of T cell subtypes in the spleen, lymph nodes and blood of IL1RL2 humanized mice, including CD4+ T cells, CD8+ T cells and Treg cells percentages are also basically the same as those of C57BL/6 wild-type mice. The results showed that humanization of IL1RL2 gene did not affect the differentiation, development and distribution of leukocytes in lymphoid tissues.

In addition, 20 8-week-old female wild-type C57BL/6 mice (+/+) and 20 IL1RL2 humanized homozygous mice (H/H) were selected, and peripheral blood was collected for complete blood cell count and blood biochemical tests. Complete blood cell count variables included: white blood cell count (WBC), red blood cell count (RBC), hematocrit (HCT), hemoglobin (HGB), mean corpuscular volume (MCV), mean corpuscular hemoglobin (MCH), mean corpuscular hemoglobin concentration (MCHC), platelet count (PLT), lymphocytes (LYMPH), monocytes (MONO), neutrophils (NEUT). Blood biochemical test variables included: alanine aminotransferase (ALT), aspartate aminotransferase (AST), albumin (ALB), blood glucose (GLU), urea (UREA), serum creatinine (CREA), cholesterol (CHOL), triglyceride (TG). The complete blood cell count results (mean value) and the blood biochemical test results are shown in the tables below.

TABLE 10
Complete blood count test results

| Variable | +/+ | H/H | Variable | +/+ | H/H |
|---|---|---|---|---|---|
| WBC (10⁹/L) | 5.23 | 6.63 | PLT (10⁹/L) | 654.55 | 716.70 |
| RBC (10¹²/L) | 9.05 | 9.97 | LYMPH (10⁹/L) | 4.36 | 5.52 |
| HGB (g/L) | 182.15 | 206.30 | LYMPH (%) | 84.42 | 83.47 |
| HCT (%) | 54.83 | 60.71 | MONO (10⁹/L) | 0.20 | 0.23 |
| MCV (fL) | 60.44 | 60.86 | MONO (%) | 3.89 | 3.62 |
| MCH (pg) | 20.08 | 20.70 | NEUT (10⁹/L) | 0.65 | 0.87 |
| MCHC (g/L) | 332.30 | 340.35 | NEUT (%) | 11.40 | 12.81 |

TABLE 11
Blood biochemical test results

| | ALT | AST | ALB | GLU | UREA | CREA | CHOL | TG |
|---|---|---|---|---|---|---|---|---|
| Group | (U/L) | (U/L) | (g/L) | (mmol/L) | (mmol/L) | (μmol/L) | (mmol/L) | (mmol/L) |
| +/+ | 38.48 | 89.44 | 47.16 | 8.79 | 7.67 | 74.79 | 2.40 | 1.06 |
| H/H | 26.97 | 72.99 | 44.33 | 8.25 | 6.40 | 78.17 | 2.33 | 1.12 |

It can be seen from the tables above that the humanization of IL1RL2 gene did not affect the composition of blood cells in mice, and the liver function of the modified mice was basically the same as that of wild-type mice.

### Example 2. Preparation of IL36A Gene Humanized Mice

Mouse IL36A gene (NCBI Gene ID: 54448, Primary source: MGI: 1859324, UniProt: Q9JLA2, located at positions 24215417 to 24225701 of chromosome 2 (NC_000068.7), based on transcript NM_019450.3 and its encoded protein NP_062323.1 (SEQ ID NO: 29)) and human IL36A gene (NCBI Gene ID: 27179, Primary source: HGNC: 15562, UniProt ID: Q9UHA7, located at positions 113005459 to 113011071 of chromosome 2 (NC_000002.12), based on transcript NM_014440.3 and its encoded protein NP_055255.1 (SEQ ID NO: 30)) are used. FIG. 9 shows a schematic comparison between mouse IL36A gene and human IL36A gene.

A nucleotide sequence encoding human IL36A protein can be introduced into the mouse endogenous IL36A locus, so that the mouse expresses human IL36A protein. Specifically, using gene editing technologies, under the control of the mouse endogenous IL36A regulatory element, the mouse humanized IL36A locus was obtained by replacing a sequence of about 8.8 kb (from mouse exon 2 to exon 5) with the corresponding human DNA sequence. The schematic diagram is shown in FIG. 10.

The targeting strategy is shown in FIG. 11. The figure shows that the targeting vector V2 contains the homology arm sequences upstream and downstream of the mouse IL36A gene, and the A2 fragment containing the human IL36A sequence. The upstream homology arm sequence (5′ homology arm, SEQ ID NO: 31) is identical to the nucleotide sequence of positions 24212600-24215846 of the NCBI accession number NC_000068.7, and the downstream homology arm sequence (3′ homology arm, SEQ ID NO: 32) is identical to the nucleotide sequence of positions 24226002-24229603 of the NCBI accession number NC_000068.7. The human IL36A sequence (SEQ ID NO: 33) in the A2 fragment is the same as the nucleotide sequences of positions 113005872-113008044 of NCBI accession number NC_000002.12. The connection between the downstream of human IL36A sequence and the mouse sequence is designed as 5′-

(SEQ ID NO: 34)
AGCCAACACTACTGACTTTGGGTTAACTATGCTGTTTTAAGGTCAGTCT
TGTAATGGGTGCTGCTTCCCAAGCAGAGAAC-3′,

where the last “A” in the sequence “TTTAA” is the last human nucleotide, and the first “G” in the sequence “GGTCA” is the first mouse nucleotide. The mRNA sequence of the modified humanized mouse IL36A is shown in SEQ ID NO:35, and the encoded protein sequence is shown in SEQ ID NO:30.

A resistance gene for positive clone selection (Neo cassette) was also included on targeting vector V2. The connection between the 5′ end of the Neo cassette and the mouse sequence is designed as 5′-

(SEQ ID NO: 36)
AGCTGGTGTAGCTAGCCAGCTTGCTCATAGAGAACTCTGTGAAGTTCCT
ATTCTCTAGAAAGTATAGGAACTTCAGGTCT-3′,

wherein the last “T” of the sequence “TCTGT” is the last mouse nucleotide, and the first “G” of the sequence “GAAGT” is the first Neo cassette nucleotide. The connection between the 3′ end of the Neo cassette and the mouse sequence is designed as 5′-

(SEQ ID NO: 37)
CCCTTCGAAGTTCCTATTCTCTAGAAAGTATAGGAACTTCCTCTACCTT
CAGAGGTATGGGATTACAAATGGGACGCACG-3′,

wherein the last “C” of the sequence “ACTTC” is the last Neo cassette nucleotide, and the first “C” of the sequence “CTCTA” is the first mouse nucleotide. In addition, a negative selection marker (the encoding gene for the A subunit of diphtheria toxin (DTA)) was also constructed downstream of the 3′ homology arm of the recombinant vector.

The construction of the targeting vector can be carried out by conventional methods, such as restriction enzyme cleavage/ligation, direct synthesis and the like. The constructed targeting vector is preliminarily verified by enzyme digestion, and then sent to a sequencing company for sequencing verification. The target vector verified by sequencing was electroporated into embryonic stem cells of wild-type mice, and the obtained cells were screened with positive clone screening marker genes. The integration of foreign genes was detected by PCR, and the correct ones were screened out. After the positive cloned cells (black mice) were selected, they were introduced into the isolated blastocysts (white mice) according to techniques known in the art, and the obtained chimeric blastocysts were transferred to the culture medium for short-term culture and then transplanted to recipient mother mice (The fallopian tubes of white mice) to produce F0 generation chimeric mice (black and white). The F0 generation chimeric mice and wild-type mice were backcrossed to obtain the F1 generation mice, and then the F1 generation heterozygous mice were bred to each other to obtain the F2 generation homozygous mice. The positive clone can also be mated with the Flp tool mouse to remove the positive clone selection marker gene, and then mated with each other to obtain the IL36A humanized homozygous mouse.

In addition, the CRISPR/Cas system can also be employed for gene editing to realize the humanization of mouse IL36A gene. FIG. 12 shows the targeting strategy. The figure shows that the targeting vector V3 contains the homology arm sequences upstream and downstream of the mouse IL36A gene, and the A3 fragment containing the human IL36A sequence. The upstream homology arm sequence (5′ homology arm, SEQ ID NO: 38) is the same as the nucleotide sequence at positions 24213960-24215846 of NCBI accession number NC_000068.7, and the downstream homology arm sequence (3′ homology arm, SEQ ID NO: 39) is the same as the nucleotide sequence at positions 24224598-24225577 of NCBI accession number NC_000068.7. The human IL36A sequence in the A3 fragment is identical to the human IL36A sequence in the A2 fragment in FIG. 11.

The construction of the targeting vector can be carried out by conventional methods, such as enzyme cleavage and ligation. The constructed targeting vector is preliminarily verified by enzyme digestion, and then sent to a sequencing company for sequencing verification. Sequencing-validated targeting vectors were used for subsequent experiments.

The target sequence determines the targeting specificity of the sgRNA and the efficiency in inducing Cas9 to cleave the target gene. Therefore, target sequence design is the key for constructing sgRNA expression vectors. sgRNA sequences that recognize the 5′ target site (sgRNA1-sgRNA5) and the 3′ target site (sgRNA9-sgRNA13) were designed and synthesized. The 5′-end target site and the 3′-end target site are located on the sequences of exons 2-3 and 5-6 of the IL36A gene, respectively. The target site sequences of each sgRNA on the IL136A gene are as follows:

sgRNA1 target site sequence (SEQ ID NO: 40):
5′-TCCTGAACATGTCTAAGCGAAGG-3′
sgRNA2 target site sequence (SEQ ID NO: 41):
5′-AGGATCTTAGTAGTCGTGTGTGG-3′
sgRNA3 target site sequence (SEQ ID NO: 42):
5′-ACCTTCGCTTAGACATGTTCAGG-3′
sgRNA4 target site sequence (SEQ ID NO: 43):
5′-CTTTCCTTGGGACTGCAGTGAGG-3′
sgRNA5 target site sequence (SEQ ID NO: 44):
5′-ACCATAATGAATAAGGAGAAAGG-3′
sgRNA9 target site sequence (SEQ ID NO: 45):
5′-CACTGACTTCGAGATGATTGTGG-3′
sgRNA10 target site sequence (SEQ ID NO: 46):
5′-GGCTGCAGACTCAAATGTAGAGG-3′
sgRNA11 target site sequence (SEQ ID NO: 47):
5′-GTTCTTGGGTCAGAATGAGTGGG-3′
sgRNA12 target site sequence (SEQ ID NO: 48):
5′-CATTAAGGTCAGTCTTGTAATGG-3′
sgRNA13 target site sequence (SEQ ID NO: 49):
5′-GAGATGATTGTGGTACATTAAGG-3′

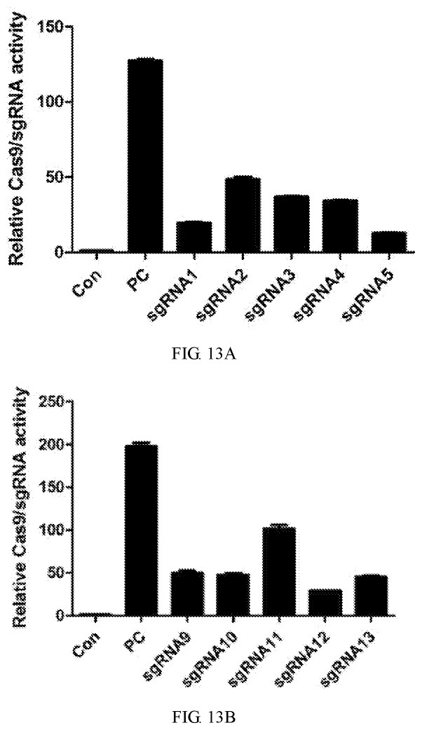

The UCA kit was used to detect the activities of the sgRNAs. The detection results are shown in the table below and FIGS. 13A-13B. From the results, it can be seen that each sgRNA has a different activity. sgRNA2 and sgRNA11 were selected for subsequent experiments.

TABLE 12
UCA test results

| 5′ end target site detection results | | 3′ end target site detection results | |
|---|---|---|---|
| Con | 1.00 ± 0.08 | Con | 1.00 ± 0.08 |
| PC | 127.37 ± 0.80 | PC | 197.38 ± 4.74 |
| sgRNA1 | 19.61 ± 0.50 | sgRNA9 | 49.81 ± 2.08 |
| sgRNA2 | 48.44 ± 1.58 | sgRNA10 | 47.42 ± 2.20 |
| sgRNA3 | 36.86 ± 0.40 | sgRNA11 | 101.21 ± 4.72 |
| sgRNA4 | 34.03 ± 0.49 | sgRNA12 | 29.04 ± 0.25 |
| sgRNA5 | 12.63 ± 0.32 | sgRNA13 | 45.14 ± 1.53 |

The forward oligonucleotide and reverse oligonucleotide (as shown in the table below) were obtained by adding enzyme cleavage sites to the 5′ end and the complementary strand, respectively. After annealing, the annealed products were ligated to the pT7-sgRNA plasmid (the plasmid was first linearized with BbsI), and the expression vectors pT7-IL36A-2 and pT7-10 IL36A-11 were obtained.

TABLE 13
List of sgRNA2 and sgRNA11 sequences
sgRNA2 sequence

| | |
|---|---|
| SEQ ID NO: 50 | Upstream: 5′-AGGATCTTAGTAGTCGTGTG-3′ |
| SEQ ID NO: 51 | Upstream: 5′-TAGGAGGATCTTAGTAGTCGTGTG-3′ |
| (forward oligonucleotide) | |
| SEQ ID NO: 52 | Downstream: 5′-CACACGACTACTAAGATCCT-3′ |
| SEQ ID NO: 53 | Downstream: 5′-AAACCACACGACTACTAAGATCCT-3′ |
| (reverse oligonucleotide) | |

sgRNA11 sequence

| | |
|---|---|
| SEQ ID NO: 54 | Upstream: 5′-GTTCTTGGGTCAGAATGAGT-3′ |
| SEQ ID NO: 55 | Upstream: 5′-TAGGGTTCTTGGGTCAGAATGAGT-3′ |
| (forward oligonucleotide) | |
| SEQ ID NO: 56 | Downstream: 5′-ACTCATTCTGACCCAAGAAC-3′ |
| SEQ ID NO: 57 | Downstream: 5′-AAACACTCATTCTGACCCAAGAAC-3′ |
| (reverse oligonucleotide) | |

The fragment DNA (SEQ ID NO: 58) containing the T7 promoter and sgRNA scaffold was synthesized by Plasmid Synthesis Company, and sequentially ligated to the scaffold vector (Takara, Cat. No. 3299) by enzyme digestion/ligation (EcoRI and BamHI). Sequencing and verification by a professional sequencing company showed that the correct pT7-sgRNA plasmid was obtained.

Fertilized eggs of mice, such as C57BL/6 mice, were taken. A microinjector was used to transfer the mixture of (1) in vitro transcription products of pT7-IL36A-2 and pT7-IL36A-11 plasmids (using Ambion in vitro transcription kits, according to the instructions), (2) the targeting vector and (3) Cas9 mRNA into the cytoplasm or nucleus of mouse fertilized eggs. The embryo microinjection was carried out according to the method described, e.g., in A. Nagy, et al., “Manipulating the Mouse Embryo: A Laboratory Manual (Third Edition),” Cold Spring Harbor Laboratory Press. The injected fertilized eggs were then transferred to a culture medium to culture for a short time and then was transplanted into the oviduct of the recipient mouse to produce the genetically modified mice (F0 generation). The mouse population was further expanded by cross-breeding and self-breeding to establish a stable IL36A humanized mouse strain.

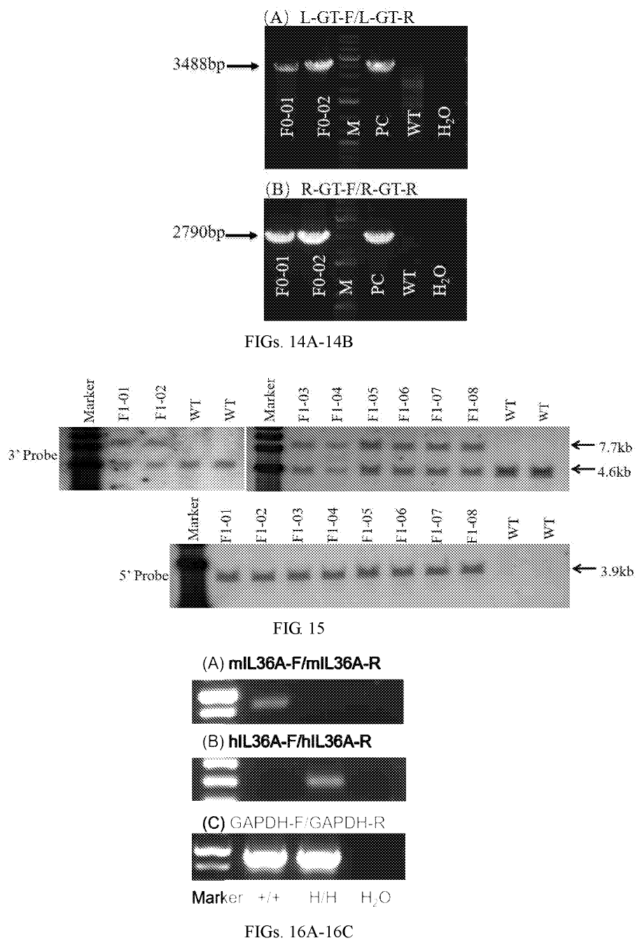

The genotype of the somatic cells of the F0 generation mice can be identified by conventional detection methods such as PCR analysis. The exemplary identification results of some of the F0 generation mice are shown in FIGS. 14A-14B. In the figure, two positive mice numbered F0-01 and F0-02 are shown. It was further verified by sequencing that these two mice were positive mice without random insertion. The primers for PCR analysis are shown in the table below.

TABLE 14
Primer names and sequences

| | | | Target |
|---|---|---|---|
| Primer | SEQ ID NO | Sequence (5′-3′) | fragment size |
| L-GT-F | SEQ ID NO: 59 | CCCTGACCACTTGCTTTTGGTGTTG | Mut: 3488 bp |
| L-GT-R | SEQ ID NO: 60 | TGAAAAGGGTCAAGAGAGTTCCTGT | |
| R-GT-F | SEQ ID NO: 61 | ATCTATGGGAGGACCAGTTTTTGAA | Mut: 2790 bp |
| R-GT-R | SEQ ID NO: 62 | TCATTGTATTCAAAAATGCAAATGAGA | |
| L-GT-F | SEQ ID NO: 59 | CCCTGACCACTTGCTTTTGGTGTTG | Mut: 2395 bp |
| Mut-R | SEQ ID NO: 63 | CTTTCACAGACCGTGGCTACTCACC | |
| Mut-F | SEQ ID NO: 64 | TCCTTGGACGTGGAATATCAGTGTGT | Mut: 1421 bp |
| R-GT-R | SEQ ID NO: 62 | TCATTGTATTCAAAAATGCAAATGAGA | |

The primer L-GT-F is located on the left side of the 5′ homology arm of IL36A, R-GT-R is located on the right side of the 3′ homology arm of IL36A, L-GT-R, R-GT-F, Mut-F and Mut-R are located on human sequences.

The IL36A humanized mice identified as positive for F0 were mated with wild-type mice to obtain F1 generation mice. F1 generation mice can be genotyped using the same PCR method, and the positive mice can be tested by Southern blot to determine the presence of random insertions. The mouse tail was cut to extract genomic DNA, and the genome was digested with BglII enzyme or AseI enzyme, transferred to a membrane, and hybridized. The 5′ Probe and 3′ Probe are located on the right side of the human sequence and the 3′ homology arm, respectively. The lengths of the specific probes and target fragments are shown in the table below.

TABLE 15
Probes and target fragment sizes

| Restriction | | Wild-type | Recombinant sequence |
|---|---|---|---|
| endonuclease | Probe | fragment size | fragment size |
| BglII | 3′Probe | 4.6 kb | 7.7 kb |
| AseI | 5′Probe | — | 3.9 kb |

Probe synthesis primers are as follows:

3′Probe-F (SEQ ID NO: 65):
5′-ACTCTCGTTTTCATACTTGAGCAGCA-3′,
3′Probe-R (SEQ ID NO: 66):
5′-GAAGAGCCTCTCCATATAAGAGAGTGAA-3′;
5′Probe-F (SEQ ID NO: 67):
5′-GCTTAAGGGAGACTGAAGGGTCAGC-3′,
5′Probe-R (SEQ ID NO: 68):
5′-CCGACTTTAGCACACATCAGGCAGA-3′.

The results of Southern blot detection are shown in FIG. 15. Based on the results of 5′ Probe and 3′ Probe, and further verification by sequencing, none of the 8 mice had random insertion. Thus, these 8 mice were positive heterozygous mice with no random insertions. Thus, IL36A humanized mice that can be stably passaged without random insertion can be constructed using this method.

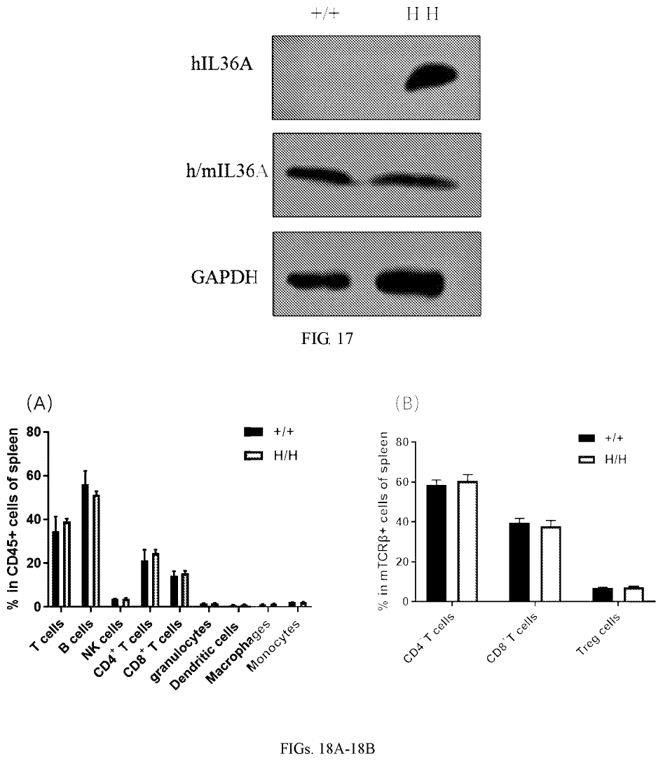

The expression of IL36A mRNA and protein in IL36A humanized mice can be detected by conventional methods. Specifically, one 8-week-old male C57BL/6 wild-type mouse and one IL36A humanized homozygous mouse prepared in this example were taken. Gastric tissues were collected after de-cervical euthanasia, and ground to prepare cell suspensions. Cellular RNA was extracted according to the TRIzol kit instructions, reverse transcribed into cDNA. RT-PCR detection was performed using the primers shown in the table below. The results are shown in FIGS. 16A-16C. As can be seen from the figure, only murine IL36A mRNA was detected in C57BL/6 wild-type mice (+/+); and only humanized IL36A mRNA was detected in IL36A humanized homozygous mice (H/H).

TABLE 16
RT-PCR primer sequences and target fragment sizes

| | | Target fragment |
|---|---|---|
| Primer and SEQ ID NO | Sequence (5′-3′) | size |
| hIL36A-F (SEQ ID NO: 76) | GACCCTTGAGAAAGACAGAGGG | Mut: 296 bp |
| hIL36A-R (SEQ ID NO: 77) | AGTCAGTAGTGTTGGCTTTCCC | |
| mIL36A-F (SEQ ID NO: 78) | AGCATCACCTTCGCTTAGACAT | WT: 409 bp |
| mIL36A-R (SEQ ID NO: 79) | TTCTTGGGTCAGAATGAGTGGG | |
| GAPDH-F (SEQ ID NO: 74) | TCACCATCTTCCAGGAGCGAGA | WT: 479 bp |
| GAPDH-R (SEQ ID NO: 75) | GAAGGCCATGCCAGTGAGCTT | |

The expression of IL36A protein in IL36A gene humanized mice was detected by Western Blot. Specifically, one 8-week-old male C57BL/6 wild-type mouse and one IL36A gene humanized homozygous mouse prepared in this example were taken. Skin tissues were collected after de-cervical euthanasia. Cross-recognizing antibody anti-IL36 alpha/IL-1F6 [EPR23152-241] (ab269274) or anti-human IL36A antibody anti-IL36 alpha/IL-1F6 [EPR23089-87] (ab269271) was used for Western Blot detection. The results are shown in the FIG. 17. As can be seen from the figure, the expression of murine IL36A protein (h/mIL36A) was detected in both C57BL/6 wild-type mice (+/+) and IL36A humanized mice (H/H). The expression of human IL36A protein (hIL36A) was detected only in IL36A humanized mice (H/H). The results showed that the IL36A humanized mice prepared by this method can successfully express human IL36A protein.

### Example 3 Drug Efficacy Verification

Thirty 9-week-old female IL1RL2 humanized homozygous mice were selected and randomly divided into control group G1, modeling group G2 and administration group G3-G5 (n=6). 2 days before the start of the experiment, the dorsal hair of the mice was removed with a shaver, exposing a skin area of 2 cm×4 cm. On the 0-5th day (at the beginning of the experiment), 5% imiquimod (IMQ) cream (10 mg/cm2) was applied to the exposed skin area of the mice in the modeling group and the administration group for psoriasis modeling. Vaseline (10 mg/cm2) was applied to the back skin area of the control group mice for 6 days. During the experiment, the G1 group was not given any drug treatment, the G2 group was intraperitoneally injected with commercially available IgG1-kappa (purchased from CrownBio, product number C0001), and the G3-G5 group was intraperitoneally injected with anti-human IL1RL2 antibody (Ab1, prepared by conventional methods). The mice in the G2-G5 group were administered twice on the Oth and 3rd day of the experiment, and the whole experimental period was 9 days. The specific dosage and administration scheme are shown in the table below.

TABLE 17
Administration scheme

| | Animal | Modeling | | | Administration | Administration | Total |
|---|---|---|---|---|---|---|---|
| Group | No. | agent | Treatment | Dosage | route | frequency | administrations |
| G1 | 6 | Vaseline | — | — | — | — | — |
| G2 | 6 | IMQ | IgG1- | 10 mg/kg | i.p. | Q3D | 2 |
| | | | kappa | | | | |
| G3 | 6 | IMQ | Ab1 | 1 mg/kg | i.p. | Q3D | 2 |
| G4 | 6 | IMQ | Ab1 | 3 mg/kg | i.p. | Q3D | 2 |
| G5 | 6 | IMQ | Ab1 | 10 mg/kg | i.p. | Q3D | 2 |

After grouping, the weights of the mice were weighed every day. The mice were photographed and the back conditions of the mice were observed and scored based on two symptoms at the skin lesions of the mice: (1) rash and (2) sesquamation. Each symptom is divided into 0-4 points according to the severity, and the PASI scoring standard is as follows: 0—none; 1—mild; 2—moderate; 3—severe; 4—extremely severe. The average of each score and the two total scores of the mice in each group were compared.

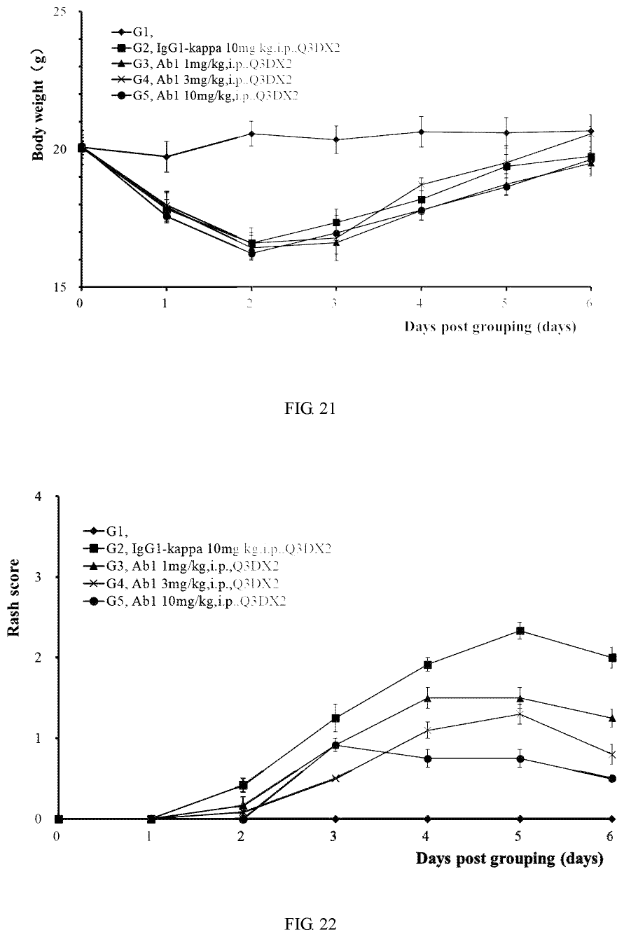

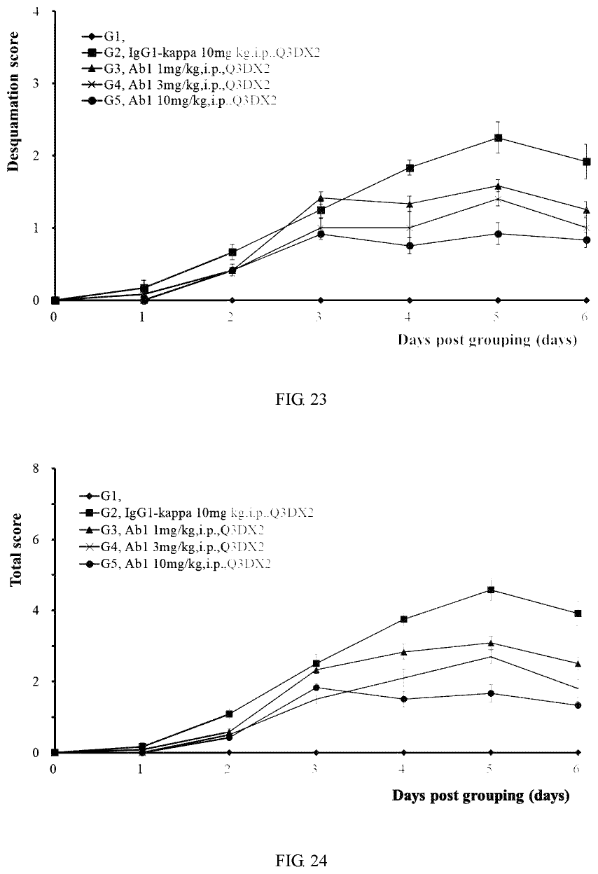

From the changes in the body weights of mice over time (FIG. 21), it can be seen that the body weights of the control group (G1) were stable throughout the experimental period. The modeling group (G2) and the treatment groups (G3-G5) had similar trends in body weight changes: the body weights first decreased to a minimum in about 2 days, and then slowly increased. During the experiment, there was little difference in body weights between the modeling group and the treatment groups. At the end of the experiment, the body weights of mice in all groups were close and there was no significant differences. The results of back skin rash, desquamation and comprehensive PASI score are shown in FIGS. 22-24. None of the mice in the control group developed any illness, while the modeling group and the administration group developed different degrees of illness. Between the model group and the administration group, the PASI scores in the administration group were significantly lower than those in the model group, indicating that the treatment of psoriasis model mice with anti-human IL1RL2 antibody had a therapeutic effect on psoriasis. The G5 group (anti-human IL1RL2 antibody Ab1 at a dose of 10 mg/kg) showed the best therapeutic effect.

The above results showed that the IL1RL2 humanized mice of the present invention can be used to establish a psoriasis model to evaluate the in vivo efficacy and dose screening of drugs targeting the human IL1RL2/IL36A signaling pathway.

### Example 4 Preparation of IL1RL2/IL36A Double Humanized Mice

The IL1RL2 humanized mice prepared in Example 1 were crossed with the IL36A humanized mice prepared in Example 2, and the positive offspring mice were screened to obtain the IL1RL2/IL36A gene double humanization mice.

### Example 5 Preparation of IL1RL2 and/or IL36A Multiple Humanized Mice

Using the above described IL1RL2 and/or IL36A humanized mice, a double or multiple humanized mouse model can also be prepared. For example, in Example 1, the embryonic stem cells used for blastocyst microinjection can be derived from mice with other genetic modifications such as PD-1, PD-L1, CD40, OX40, IL6, IL12, IL23, TNF-α, etc. Alternatively, on the basis of humanized IL1RL2 and/or IL36A mice, mouse embryonic stem cells can be isolated and gene recombination targeting technology can be used to obtain IL1RL2 and/or IL36A and other genetically modified double-gene or multi-gene modifications mouse models. The IL1RL2 and/or IL36A mouse homozygote or heterozygote obtained by this method can also be mated with other gene-modified homozygous or heterozygous mice, and the offspring are screened. According to Mendelian inheritance, there is a certain probability to obtain IL1RL2 and/or IL36A humanized and other genetically modified double-gene or multi-gene modified heterozygous mice, which can be then mated with each other to obtain double-gene or multi-gene modified homozygotes.

Using the above-mentioned mice, various human disease models, including psoriasis and multiple sclerosis can be induced and prepared. These model mice can be used to test the in vivo efficacy of human-specific antibodies. For example, IL1RL2 and/or IL36A gene humanized mice can be used to evaluate the pharmacodynamics, pharmacokinetics, and in vivo therapeutic efficacy of human-specific IL36 signaling pathway drugs in various disease models known in the art.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric Interleukin-36 alpha (IL36A).

2. The animal of claim 1, wherein the sequence encoding the human or chimeric IL36A is operably linked to an endogenous regulatory element (e.g., endogenous 5′ UTR and/or 3′ UTR) at the endogenous IL36A gene locus in the at least one chromosome.

3. The animal of claim 1 or 2, wherein the sequence encoding a human or chimeric IL36A comprises a sequence encoding an amino acid sequence that is at least 70%, 75%, 80%, 85%, 90%, 95%, 99%, or 100% identical to human IL36A (SEQ ID NO: 30).

4. The animal of any one of claims 1-3, wherein the animal is a mammal, e.g., a monkey, a rodent, a mouse, or a rat.

5. The animal of any one of claims 1-4, wherein the animal is a mouse.

6. The animal of any one of claims 1-5, wherein the animal does not express endogenous IL36A or expresses a decreased level of endogenous IL36A as compared to IL36A expression level in a wild-type animal.

7. The animal of any one of claims 1-6, wherein the animal has one or more cells expressing human or chimeric IL36A.

8. The animal of any one of claims 1-7, wherein the animal has one or more cells expressing human or chimeric IL36A, and the expressed human or chimeric IL36A can bind to endogenous IL36A receptor (IL1RL2), inducing downstream signaling pathways.

9. The animal of any one of claims 1-7, wherein the animal has one or more cells expressing human or chimeric IL36A, and the expressed human or chimeric IL36A can bind to human or humanized IL36A receptor (IL1RL2), inducing downstream signaling pathways.

10. A genetically-modified, non-human animal, wherein the genome of the animal comprises a replacement of a sequence encoding a region of endogenous IL36A with a sequence encoding a corresponding region of human IL36A at an endogenous IL36A gene locus.

11. The animal of claim 10, wherein the sequence encoding the corresponding region of human IL36A is operably linked to an endogenous regulatory element at the endogenous IL36A locus, and one or more cells of the animal express a human or chimeric IL36A.

12. The animal of claim 10 or 11, wherein the animal does not express endogenous IL36A or expresses a decreased level of endogenous IL36A as compared to IL36A expression level in a wild-type animal.

13. The animal of any one of claims 10-12, wherein the replaced sequence encodes the full-length protein of IL36A.

14. The animal of any one of claims 10-13, wherein the animal is a mouse, and the replaced endogenous IL36A region comprises a portion of exon 2, exon 3, exon 4, and/or a portion of exon 5 of the endogenous mouse IL36A gene.

15. The animal of any one of claims 10-14, wherein the animal is heterozygous or homozygous with respect to the replacement at the endogenous IL36A gene locus.

16. A non-human animal comprising at least one cell comprising a nucleotide sequence encoding a human or humanized IL36A polypeptide, wherein the human or humanized IL36A polypeptide comprises at least 50, 100, 110, 120, 130, 140, 150, 155, 156, 157, or 158 contiguous amino acid residues that are identical to the corresponding contiguous amino acid sequence of a human IL36A, wherein the animal expresses the human or humanized IL36A polypeptide.

17. The animal of claim 16, wherein the nucleotide sequence is operably linked to an endogenous IL36A regulatory element of the animal.

18. The animal of claim 16 or 17, wherein the nucleotide sequence is integrated to an endogenous IL36A gene locus of the animal.

19. The animal of any one of claims 16-18, wherein the human or humanized IL36A polypeptide has at least one mouse IL36A activity and/or at least one human IL36A activity.

20. A method for making a genetically-modified, non-human animal, comprising:
replacing in at least one cell of the animal, at an endogenous IL36A gene locus, a sequence encoding a region of an endogenous IL36A with a sequence encoding a corresponding region of human IL36A.

21. The method of claim 20, wherein the sequence encoding the corresponding region of human IL36A comprises a portion of exon 1, exon 2, exon 3, and a portion of exon 4 of a human IL36A gene.

22. The method of claim 20 or 21, wherein the sequence encoding the corresponding region of human IL36A comprises at least 50, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 474, or 477 nucleotides of exon 1, exon 2, exon 3, and/or exon 4 of a human IL36A gene.

23. The method of any one of claims 20-22, wherein the sequence encoding the corresponding region of human IL36A encodes a sequence that is at least 80%, 85%, 90%, 95%, 96%, 97%, 98%, 99%, or 100% identical to SEQ ID NO: 30.

24. The method of any one of claims 20-23, wherein the animal is a mouse, and the locus is a portion of exon 2, exon 3, exons 4, and/or a portion of exon 5 of the mouse IL36A gene.

25. A method of making a genetically-modified non-human animal cell that expresses a human or chimeric IL36A, the method comprising:
replacing, at an endogenous mouse IL36A gene locus, a nucleotide sequence encoding a region of endogenous IL36A with a nucleotide sequence encoding a corresponding region of human IL36A, thereby generating a genetically-modified non-human animal cell that includes a nucleotide sequence that encodes the human or chimeric IL36A, wherein the animal cell expresses the human or chimeric IL36A.

26. The method of claim 25, wherein the animal is a mouse.

27. The method of claim 25 or 26, wherein the nucleotide sequence encoding the human or chimeric IL36A is operably linked to an endogenous IL36A regulatory region, e.g., a promoter.

28. The animal of any one of claims 1-19, wherein the animal further comprises a sequence encoding an additional human or chimeric protein, e.g., IL36A receptor (IL1RL2), IL33, IL7R, IL6, IL12, IL23, or Tumor necrosis factor alpha (TNF-α).

29. The animal of claim 28, wherein the additional human or chimeric protein is IL1RL2.

30. The method of any one of claims 20-27, wherein the animal or mouse further comprises a sequence encoding an additional human or chimeric protein, e.g., IL1RL2, IL33, IL7R, IL6, IL12, IL23, or TNF-α.

31. The method of claim 30, wherein the additional human or chimeric protein is IL1RL2.

32. A genetically-modified, non-human animal whose genome comprises at least one chromosome comprising a sequence encoding a human or chimeric interleukin-1 receptor-like 2 (IL1RL2).

33. The animal of claim 32, wherein the sequence encoding the human or chimeric IL1RL2 is operably linked to an endogenous regulatory element at the endogenous IL1RL2 gene locus in the at least one chromosome.

34. The animal of claim 32 or 33, wherein the sequence encoding a human or chimeric IL1RL2 comprises a sequence encoding an amino acid sequence that is at least 70%, 75%, 80%, 85%, 90%, 95%, 99%, or 100% identical to human IL1RL2 (SEQ ID NO: 2).

35. The animal of claim 32 or 33, wherein the sequence encoding a human or chimeric IL1RL2 comprises a sequence encoding an amino acid sequence that is at least 70%, 75%, 80%, 85%, 90%, 95%, 99%, or 100% identical to amino acids 1-356 of human IL1RL2 (SEQ ID NO: 2).

36. The animal of claim 32 or 33, wherein the sequence encoding a human or chimeric IL1RL2 comprises a sequence encoding an amino acid sequence that is at least 70%, 75%, 80%, 85%, 90%, 95%, 99%, or 100% identical to SEQ ID NO: 10.

37. The animal of any one of claims 32-36, wherein the animal is a mammal, e.g., a monkey, a rodent, a mouse, or a rat.

38. The animal of any one of claims 32-37, wherein the animal is a mouse.

39. The animal of any one of claims 32-38, wherein the animal does not express endogenous IL1RL2 or expresses a decreased level of endogenous IL1RL2 as compared to IL1RL2 expression level in a wild-type animal.

40. The animal of any one of claims 32-39, wherein the animal has one or more cells expressing human or chimeric IL1RL2.

41. The animal of any one of claims 32-40, wherein the animal has one or more cells expressing human or chimeric IL1RL2 that can bind to endogenous IL36A to induce downstream signaling pathways.

42. The animal of any one of claims 32-40, wherein the animal has one or more cells expressing human or chimeric IL1RL2 that can bind to human IL36A to induce downstream signaling pathways.

43. A genetically-modified, non-human animal, wherein the genome of the animal comprises an insertion of a sequence encoding a human or chimeric IL1RL2 at an endogenous IL1RL2 gene locus.

44. The animal of claim 43, wherein the sequence encoding a human or chimeric IL1RL2 is operably linked to an endogenous regulatory element at the endogenous IL1RL2 locus, and one or more cells of the animal express the human or chimeric IL1RL2.

45. The animal of claim 43 or 44, wherein the animal does not express endogenous IL1RL2 or expresses a decreased level of endogenous IL1RL2 as compared to IL1RL2 expression level in a wild-type animal.

46. The animal of any one of claims 43-45, wherein the sequence encoding a human or chimeric IL1RL2 is inserted within exon 1, intron 1, exon 2, intron 2, exon 3, intron 3, exon 4, intron 4, exon 5, intron 5, exon 6, intron 6, exon 7, intron 7, exon 8, intron 8, exon 9, intron 9, exon 10, intron 10, or exon 11 of endogenous IL1RL2 gene.

47. The animal of any one of claims 43-46, wherein the sequence encoding a human or chimeric IL1RL2 is inserted within exon 2 of endogenous IL1RL2 gene.

48. The animal of any one of claims 43-47, wherein the sequence encoding a human or chimeric IL1RL2 is inserted immediately after a nucleotide corresponding to position 238 of NM 133193.4.

49. The animal of any one of claims 43-48, wherein the inserted sequence comprises, optionally from 5′ end to 3′ end:
a) a sequence encoding a signal peptide;
b) a first sequence comprising a sequence encoding a humanized IL1RL2 extracellular region and a humanized IL1RL2 transmembrane region;
c) a second sequence comprising a sequence encoding all or a portion of the cytoplasmic region of an endogenous IL1RL2;
d) a regulatory sequence of endogenous IL1RL2 gene (e.g., 3′ UTR); and
e) optionally one or more auxiliary sequences (e.g., WPRE, lox2, STOP, and/or polyA).

50. The animal of claim 49, wherein the sequence encoding a signal peptide encodes an amino acid that is at least 70%, 75%, 80%, 85%, 90%, 95%, 99%, or 100% identical to amino acids 1-19 of SEQ ID NO: 2.

51. The animal of claim 49 or 50, wherein the first sequence further comprises a sequence encoding at least 1, at least 2, at least 3, at least 4, at least 5, or at least 6 amino acids of the transmembrane region of a humanized IL1RL2.

52. The animal of any one of claims 49-51, wherein the first sequence is at least 70%, 75%, 80%, 85%, 90%, 95%, 99%, or 100% identical to SEQ ID NO: 5, and the second sequence is at least 70%, 75%, 80%, 85%, 90%, 95%, 99%, or 100% identical to SEQ ID NO: 6.

53. The animal of any one of claims 49-52, wherein the first sequence encodes an amino acid that is at least 70%, 75%, 80%, 85%, 90%, 95%, 99%, or 100% identical to amino acids 1-356 of SEQ ID NO: 2, and the second sequence encodes an amino acid sequence that is at least 70%, 75%, 80%, 85%, 90%, 95%, 99%, or 100% identical to amino acids 360-574 of SEQ ID NO: 1.

54. The animal of any one of claims 49-53, wherein the one or more auxiliary sequences comprise a PolyA sequence.

55. The animal of any one of claims 43-54, wherein the animal is heterozygous or homozygous with respect to the insertion at the endogenous IL1RL2 gene locus.

56. A non-human animal comprising at least one cell comprising a nucleotide sequence encoding a humanized IL1RL2 polypeptide, wherein the humanized IL1RL2 polypeptide comprises at least 50 contiguous amino acid residues that are identical to the corresponding contiguous amino acid sequence of a human IL1RL2, wherein the animal expresses the humanized IL1RL2 polypeptide.

57. The animal of claim 56, wherein the humanized IL1RL2 polypeptide has at least 50, 100, 150, 160, 170, 180, 190, 200, 210, 220, 230, 240, 250, 260, 270, 280, 290, 300, 310, 320, 330, 340, 350, 351, 352, 353, 354, 355, or 356 contiguous amino acid residues that are identical to the corresponding contiguous amino acid sequence of human IL1RL2 extracellular and transmembrane regions.

58. The animal of claim 56 or 57, wherein the humanized IL1RL2 polypeptide comprises a sequence that is at least 90%, 95%, or 99% identical to amino acids 1-356 of SEQ ID NO: 2.

59. The animal of any one of claims 56-58, wherein the nucleotide sequence is operably linked to an endogenous IL1RL2 regulatory element of the animal.

60. The animal of any one of claims 56-59, wherein the nucleotide sequence is integrated to an endogenous IL1RL2 gene locus of the animal.

61. The animal of any one of claims 56-60, wherein the humanized IL1RL2 polypeptide has at least one mouse IL1RL2 activity and/or at least one human IL1RL2 activity.

62. A method of making a genetically-modified non-human animal cell that expresses a chimeric IL1RL2, the method comprising:
inserting at an endogenous IL1RL2 gene locus (e.g., exon 2 of endogenous IL1RL2 gene), a nucleotide sequence comprising, optionally from 5′ end to 3′ end:
a) a first sequence comprising a sequence encoding all or a portion of the extracellular region and transmembrane region of a human IL1RL2;
b) a second sequence comprising a sequence encoding all or a portion of the cytoplasmic region of an endogenous IL1RL2;
c) a regulatory sequence of endogenous IL1RL2 gene (e.g., 3′ UTR); and
d) optionally one or more auxiliary sequences (e.g., WPRE, lox2, STOP, and/or polyA);
thereby generating a genetically-modified non-human animal cell that includes a nucleotide sequence that encodes the chimeric IL1RL2, wherein the animal cell expresses the chimeric IL1RL2.

63. The method of claim 62, wherein the animal is a mouse.

64. The method of claim 62 or claim 63, wherein the nucleotide sequence encoding the chimeric IL1RL2 polypeptide is operably linked to an endogenous IL1RL2 regulatory region, e.g., promoter.

65. The animal of any one of claims 32-61, wherein the animal further comprises a sequence encoding an additional human or chimeric protein, e.g., IL36A, IL33, IL7R, IL6, IL12, IL23, or Tumor necrosis factor alpha (TNF-α).

66. The animal of claim 65, wherein the additional human or chimeric protein is IL36A.

67. The method of any one of claims 62-64, wherein the animal or mouse further comprises a sequence encoding an additional human or chimeric protein, e.g., IL36A, IL33, IL7R, IL6, IL12, IL23, or TNF-α.

68. The method of claim 67, wherein the additional human or chimeric protein is IL36A.

69. A method of determining effectiveness of a therapeutic agent for treating an immune disorder, comprising:
a) administering the therapeutic agent to the animal of any one of claims 1-19, 28, 29, 32-61, 65 and 66, wherein the animal has the immune disorder; and
b) determining effects of the therapeutic agent in treating the immune disorder.

70. The method of claim 69, wherein the immune disorder is psoriasis.

71. The method of claim 70, wherein the psoriasis is induced by treating the animal with imiquimod (IMQ).

72. The method of claim 70 or 71, wherein the effects are evaluated by comparing skin damage parameters (e.g., rash and sesquamation) of the animal with an animal that is not treated with the therapeutic agent.

73. The method of any one of claims 69-72, wherein the therapeutic agent is an anti-IL36A antibody, an anti-IL1RL2 antibody, and/or a corticosteroid (e.g., dexamethasone).

74. A method of determining effectiveness of a therapeutic agent for reducing an inflammation, comprising:
a) administering the therapeutic agent to the animal of any one of claims 1-19, 28, 29, 32-61, 65 and 66, wherein the animal has the inflammation; and
b) determining effects of the therapeutic agent for reducing the inflammation.

75. A method of determining effectiveness of a therapeutic agent for treating an autoimmune disease, comprising:
a) administering the therapeutic agent to the animal of any one of claims 1-19, 28, 29, 32-61, 65 and 66, wherein the animal has the autoimmune disease; and
b) determining effects of the therapeutic agent for treating the autoimmune disease.

76. The method of claim 75, wherein the autoimmune disease is inflammatory arthritis, eczema, eosinophilic esophagitis, rheumatoid arthritis, Crohn's disease, systemic lupus erythematosus, ankylosing spondylitis, inflammatory bowel diseases (IBD), ulcerative colitis, multiple sclerosis, systemic juvenile idiopathic arthritis (SJIA), and/or scleroderma.

77. The method of any one of claims 74-76, wherein the therapeutic agent is an anti-IL36A antibody, an anti-IL1RL2 antibody, or a corticosteroid (e.g., dexamethasone).

78. A method of determining toxicity of a therapeutic agent comprising:
a) administering the therapeutic agent to the animal of any one of claims 1-19, 28, 29, 32-61, 65 and 66; and
b) determining effects of the therapeutic agent to the animal.

79. The method of claim 78, wherein the therapeutic agent is an anti-IL36A antibody or an anti-IL1RL2 antibody.

80. The method of claim 78 or 79, wherein determining effects of the therapeutic agent to the animal involves measuring the body weight, red blood cell count, hematocrit, and/or hemoglobin of the animal.

81. A protein comprising an amino acid sequence, wherein the amino acid sequence is one of the following:
(a) an amino acid sequence set forth in SEQ ID NO: 1, 2, 10, 29, or 30;
(b) an amino acid sequence that is at least 90% identical to SEQ ID NO: 1, 2, 10, 29, or 30;
(c) an amino acid sequence that is at least 91%, 92%, 93%, 94%, 95%, 96%, 97%, 98%, or 99% identical to SEQ ID NO: 1, 2, 10, 29, or 30;
(d) an amino acid sequence that is different from the amino acid sequence set forth in SEQ ID NO: 1, 2, 10, 29, or 30 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 1, 2, 10, 29, or 30.

82. A nucleic acid comprising a nucleotide sequence, wherein the nucleotide sequence is one of the following:
(a) a sequence that encodes the protein of claim 81;
(b) SEQ ID NO: 3, 4, 5, 6, 7, 8, 9, 11, 12, 31, 32, 33, 34, 35, 36, 37, 38, or 39;
(c) a sequence that is at least 90% identical to SEQ ID NO: 3, 4, 5, 6, 7, 8, 9, 11, 12, 31, 32, 33, 34, 35, 36, 37, 38, or 39; and
(d) a sequence that is at least 91%, 92%, 93%, 94%, 95%, 96%, 97%, 98%, or 99% identical to SEQ ID NO: 3, 4, 5, 6, 7, 8, 9, 11, 12, 31, 32, 33, 34, 35, 36, 37, 38, or 39.

83. A cell comprising the protein of claim 81 and/or the nucleic acid of claim 82.

84. An animal comprising the protein of claim 81 and/or the nucleic acid of claim 82.

85. A method for making a genetically-modified, non-human animal, comprising:
(1) providing a plasmid comprising a human or chimeric IL1RL2 gene fragment, flanked by a 5′ homology arm and a 3′ homology arm, wherein the 5′ and 3′ homology arms target an endogenous IL1RL2 gene; and
(2) modifying the genome of a fertilized egg or an embryonic stem cell by inserting the human or chimeric IL1RL2 gene fragment into the genome.

86. The method of claim 85, wherein the 5′ homology arm comprises SEQ ID NO: 3 and the 3′ homology arm comprises SEQ ID NO: 4.

87. A method for making a genetically-modified, non-human animal, comprising:
(1) providing a plasmid comprising a human or chimeric IL36A gene fragment, flanked by a 5′ homology arm and a 3′ homology arm, wherein the 5′ and 3′ homology arms target an endogenous IL36A gene; and
(2) modifying the genome of a fertilized egg or an embryonic stem cell by inserting the human or chimeric IL36A gene fragment into the genome.

88. The method of claim 87, wherein the 5′ homology arm comprises SEQ ID NO: 31 and the 3′ homology arm comprises SEQ ID NO: 32.


SEQUENCE LISTING (81 sequences)

SEQ ID NO: 1 - protein, 574 aa, Mus musculus
MGVTSLLFCGVFFLLLLFVAADTCEDIFMHNVIISEGQPFPFNCTYPPETNGAVNLTWYKTPSKSPVSNNRHLRVHQDQTWILFLPLTLEDSGIYQCVIRNAHNCYQIAVNLTVLKNHWCDSSMEGSPVNSPDVYQQILPIGKSGSLNCHLYFPESCALDSIKWYKGCEEIKAGKKYSPSGAKLLVNNVAVEDGGSYACSARLTHLGRHFTIRNYIAVNTKEVEYGRRIPNITYPKNNSIEVPLGSTLIVNCNITDTKENTNLRCWRVNNTLVDDYYKDSKRIQEGIETNVSLRDQIRYTVNITFLKVKMEDYGRPFTCHAGVSAAYIILIYPVPDFRAYLLGGLMAFLLLVVSVLFIYNSFKIDIMLWYRSAFHTAQAPDDEKLYDAYVLYPKYPRGSQGHDVDTLVLKILPEVLEKQCGYKLFIFGRDEFPGQAVASVIDENIKLCRRLMVFVAPESSSFGFLKNLSEEQIAVYNALIQHGMKVILIELEKVKDYSTMPESIQYIRQKHGAIQWDGDFTEQSQCAKTKFWKKVRYHMPPRRYPASSPVQLLGHIPCNCKAGKCNAATGLITP

SEQ ID NO: 2 - protein, 575 aa, Homo sapiens
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 3 - protein, 3949 aa, synthetic construct
CCCTAGAATATGGAGACGGACAGAGCTTCCTCTCCAAATCAGAAATACCTTGTCAGAGCTGGTAATGACCTGGGGCCAGGGCCTTGGGGGGAGCTGTGACCTCAGATCCTGAGATCTCAACTCTTCTGACTGTATGGAGTTATTGGTTATCAGTCTCAAGGCCATTGTATGCATGGCCTATATATAGTATTCTCCAGAAACCCTGTGTTCCTCTCTTGCAACACTGTTTGATTAGCTTCCTGCTGACTTTGTCCCGTTGTCGGGTAGCTTCTTTGGACACCCCCATTTCTCCCCTGGAAGTTGTACATCAAATAATGCTGTACTTTTTAAATAAGCTTGCTTGGCTCAAGCCATAGCTTGTACAAGACCTCCAGATCCCAAACTTTTTTTTTAAATCTTCTCTCTCTTCTATCCTCTTTATCTTCTGATCTCCTGGTTCTCTCTCTCTCTCTCTCTCTCTCTCTCTCTCTCTCTCTCTCCCTCTCTCCCTCTCTCCCTCTCTCTCTCTCTCTCTCTCTCTCTCTCTCCCCTGTTACTGAAGAAGGGCTTGCTGGTCTAGGCCCTAACCCCCACTCTCTGTGTCTCAGGGGAACACAGCAGCCATGAGCTCAGTAGCACACACCGACTTGCCCTGCCTTTGTGTATTATTAGATCTACTCTTTCCCCATGCAACTCCCCCGCCCCCATGCCTTCACCCTGCCATTCCTCTTCAGCTCTATGTGCACCTGGTTTGAAAACCTGTGCTGTTCCCTTAGTGCTTTGCGCTTTAGGCTGTCTTTAGTGGTAAGGGTACCAATTCCGGGGTGAGGAGGAATTAGTGTGAGATTATATTTTTCCTGATTTAAACATTGTAATATCTTTTAATTTTGCCTATAATAATGGCAGCCATTGTATTGTCGTTCCCAGGCCCCATGTGATCTAGCCACACCTACTTCCATACTTCAGTTTACCCAGCTCTCCCCTCTGCTGTGTGGCAATCACTCTCCCTTCATTTCAGTTTTTTTTTTTGGAGTGTCCCAAACTCGGTCCTTCAGAGACTTTGTACTGACGGTTCATTCTGTAGGGATGTAAATGGCTGTTGCAGAAGATCAGACTTTGGTCCCCAGAACCCATATCAGGTAACTCACAAACACCTGTAATTCCAGCTCCAAGGGAGCCGACACCCTCTTCTGGACCCTATGGTCACCTGCAGTCACATGCACATAGCCAGTGCAGATGCACACACATAATTAAACATAGTAAAAATAAAACGTAAAAAGTAGAAAGAGAGAGCTCTTGCCCCAGACATCTACCTGTTGGGCTCCAGGCCACTGCTCAGGAGAAAGGCTGTGCCTTATCATATTCTTTCCACCTGTCTCCTAACTGTATTTCATATCTTATGTGCTTCCTAATAGTTGAATATCTCTTTTAAAAAAATGTTATTTACCAGAAAATCATGGTGGCAAGCTGTTGAAGGGCTCATGTCTCTAAGCTGTCTTCCCAACACTCAGTGTGCCATCGGACACATTCGGATGCCTTTAATATTTGGCAAGAACATGGGGCATCAGAATCCTTCTCTAGAGAATGGTGTCATGGAATAATCAGTCAGAGGTTTGCCAACGCAGACGAGGGCACACCTACCTAATGATCCAGGAGCTTCTCTCAGACGTGTAAGAGACATTGCCAGGATTGTCGCGGCAACATTATGTATAATAATTAGCAAAACGTAAAAAAAATGGGACAACCAAAATGGCTAAGGGAGAGAGACACGTCGGTTGCTCATGCTCACAGGATGGGGCAAGCCTTTATTGAGAAGGACCACACCTCAAATGTTTAAATCCCGCAAGTATAATGCTTTTATTAAAAAGTAAGTTATGTAAGTGCACAGTATAGTAAATTTTATACAGTGACTAGGAAATCTTATGTAAGAATATCTTACATTGCTTAAGGAATTTCCTGCTTCTCCCTCTCTGCTCTCCACCCCCACACACCACTCAGACACACACAAAGGGAGGGGGAGGGGAGAGAATAAGCACCAAGCTGAAGACTGGGAGTTTGGGTTTGGGCGGACAGAGACCGGAAGCTGGGAGATGATGGCTTCTGATGTTTGTTGCTTACGCGACACAGCTTGTTTGGAATTATGTGGCCCTTGACAACTGCGTGGCACACACTTCAAGATACAAAAGTGAGAAGAAGAGGGGGGTCACCAGAGGAGACTGAAGTCAGTAAGTTTCTACCAAAGCTGGGTTTCTTCTGGCCACTCCAAACAAAGCTGAAATTGTCCTTCAGACCTCTGTGAGTACAGTCTCAGTTCCTTCCGTAGTTCTCTGGGAGGTTCTGCGTGCACACACGTGATAGTTTTGTGTTTTTATTTTTAAAACTTCAATCAGTTTTGTCTTAATTTTATATATTACTGGGGTTTGGTGCGCACTAATTGAACCCAGCTTCTTTTCACCGGCTTCATCCACCCACCCCTGTAATCTCTCTTCTACATCTTAGCTTCCTGTTTGTTTAATTTCCGTATAGGTAGGACGTTTCTTCTGTTTCTCATTCCGCTTAACGTCGTGGTTTTTGTTTTTGTTTTCCCTGAAAACTCACTATCCCCATATCTGTTCTGTATCCAGAACTAATGATTGATTCCTCATGCATCAGCAGACTTACTCAACACAGAGATCATCCCAGAATTGTAAGGCAGAGTGCTTCTGCCAAGGGACAGTACTCGGACAGACACGTGTACATCTTAACTTTGTGAACCTGGGACAGCTAGTCAGACTGACGCTGTGGTTGTTTTCAAAGTCTTACAGTTTCTAATCCACAAAGACTCCGAGGGATGCCCACTAAGAAGCTGTTGGTCTGACAGCGTTTGCCGCCACTGCTCTTCTCAGTGCCTAGTGGGGTCTCCAGTGTCTCTGGCGCAGGAAGACAGGTCCGAGCAAGCATTACTTTATGGCAAGTTTAGACTGACTGTATCGGTGCTCAACCTGTCGGGAGTCTGCTTTACCTAAGATCTGGCACGAGGACGGGGCTGGGGTGGGGTTAATGCCTTTTCTCAGAAGCGCTTCCACCTCTGGAATAGTCCATTTTAGGACAGTGCTGGGTGTGTAAGAACCGCTCAGATAGTGTTTGGAAAGGATATAATAATTTTTAGCGATGATCTGGAGCATATTTAAGCCGACATTCCTGATAGATGAACGTGCTGGCCTGGGATCAGGAAAAAAAGACTGAACGATTTTCGAAGGACGGATTATTTCTGTCCTGCTCCACTTGGGGAAGGTGAGTCTGTTCCGCCCGCGGCGGAGAAGCGATGCCCAAGGTGCAGAGTGCGGTGACCAACCAGGATTCGGTCCCCAGAGTGGTTCCCGCTCTGCTCCACGAGACCCAGCGCTCTGAGACCCCATAGGCAGGTGGAGCAAGCCCGGGGCAGCCGGAGCGGAGGGAGTTAGCGATCAATAGTCACTGGGGCGGCTAGAAGACTCCGACCTGGAAAGTGGGCCGGGACTTGGGTCAAACTCTGGGATTCCTGCTCTCCCTTGCCCTTTCCAGGTGGGCCAGCACTTACCGCTCAGCCAGCGAGCACATAGGGTTACAGGGGAGCCCCTCCGACCCATGCGATCCCAGATGCACGCTGGGCGGGTCCACAGTCTGCTCTGGCTGGGGTTCAGTCTCCTGGCCCTGGCTTGATCCAAGTGCCAGCACATCTGGCCCTGTCTCCCAACTCCCCACCAGTCCCCTTCACAGGCCAGTCGGCGTTTACTGGCTTCTGAAGGTCTGGTGTAATGGAGATGGGGAAAGGCTGGCCTTGGAACGGAGGGAGGCCAAGTGGAGGGCTCCCCATGCAGAGGGGAAGGAAGGCCAGTGACCTGAACTGGGACTGACATTCTGGATTCTCTAAAATTGTGTGCGGGAAGGTACCAGCCTCACTGCATCCCTTACTTCTACCATTGTCCTCTCTTTTCCTTTGTAGCCTGAG

SEQ ID NO: 4 - DNA, 4271 bp, synthetic construct
  source 1..4271: mol_type = other DNA
ggggttacatctttgctcttctgtggggtgtttttcctgcttctgcttttcgtggcagcaggtaagttctgcgggtctgtgagtccttgtatcccagagacctgttgccttgccagcagcatcctttgtcattcatctccgtgcccctgactcacccacttgcaggggcatcaactaagccccaaccttgctagtcactctttccaccagataatctggcaggccggtcaaaaagggagcggggagggagggataggaaaagttcattcccaacttcttggaggctcaacccccaaatgtcaccttggaactttggactaacaggtgatacagagaagtcctcaaacactcgagtggactcagttccaatgcttaaacagatgggctactagtgtcatctatttcatataaagggctattttatggtccaaaactaccagagagtacaagtttctaagaaaacccaacttggtagaaatgggctttaaagggaagctgtctttagaaccaagcactgtggaacgaggatacatctgactgcaagtataggcaggaaaacgaccaggttggttctatgttgagacagagactaagggcagaagagtgaacagatacagacacaggggccgtggttctgtggagggtcctggggaagccttgtcctcactatacatttgaaggtcttagcattatgcttctaaatgactgggctagtttggggaaacaccccaaataaacaaactcctaaatgcaatttcacattgaccagagcaaaatattccctaaaaagaataatgactccagttttctgcctttattttattttattttattttattttattttattttattgttttcctttccttccccagatacgtgtgaggacatttttatgcacaatgtgataatttcagagggccagccttttcctttcaactgcacatacccgccagaaacaaacggggcagtaaatctgacatggtacaaaacacctagcaaaagcccagtatctaacaacagacaccttagagttcaccaggaccagacctggatcttgtttcttccattgacactggaggactccggtatctatcagtgtgttataaggtaagtccttcatttaaagtggaactaattcccaagtctccctctccatttctaaagacaactttttaagaaaatgggtattattggcctttattttggagacttgttatatagcctagacaagtttccaactgctttctaaagtactgaaaggacaggtgtgaaccaccacaaccagccgctacacaccacaaccagctgctacacaaatgcagggcagccttgaactggcaggagatgacgtccagtgagcacttttgaaagcagaacagctttagaaataacttttggagcacagattgcaccatcttcttcattgaagttttagcccagaccagcttacagagagaggtgttttatcaatgagattgctgccatttccgaagatggctaatggttttggaaaactggctgatccagacccaactagatcattcaaacacatctgataaggaatcagcttaaggcagacatttgaaagctagcaaatctgggagatgttctagtgtctacatgaatccatgtgccagaggtaatgtttttgagtggcagtggactactgaactatttccatggaagacaattttgcttggagagtactagagtctgctagaaaatacgtgccccaccaaaggcacttcttcctgtccccagtctgtctctgtctgtctgtctgtctgtctctttccctgtctctgtcttgctctgcttgtctctccctagttctctcccttcccttctcttttccttttcagtttgtaaagattttcaagagttcaataaaacccacctgtaaaactcatttggcctgagaattaaaattctctgactttccacataagcttgactgacaacaatgtattatgtactctctctaaaccttgcctttcccacctgtaaattgggtgtgttctgtacgcctttaaactttgttcacctggagcagcttttagtgattaggttccatgctgtcgacagttttaagggatacaggcagtctcacagatcagatgtgtataagtgtaggtgacttctgtaccagaagtgaagtgtaagcactgtatggagtcttcagataaatagttcaagaggttcatggtggccctcagtggctcctgggccgtgctgaatgagacaccagtgtctaccctgatgaataccacttatccttggttagtgcatggtttgagggttggttattctttacttcatatgaggcagagggtttgtgcagtgtctagaaaataataagaaaagatctgaaaatagctgactgaattctgagactgcttaagagttcattggctgcattctgctgtgtttcattgttattttgcttgtttttgttttgttttgttttacaggaatgcccacaactgctaccaaatagctgtgaacctaaccgttttaaaaaaccactggtgtgactcttccatggaggggagtcccgtaaattcaccagatgtgtaccagcaaatattacccataggaaaatcgggcagtctgaattgtcatctctacttcccagaaagttgtgctttggattcaataaaatggtataaggtaaaaccaaacccaaacccaaaccaaacaaaaccaaaccaacaacaataacaaaacaacaatgaaatcctctattgataatgttttctcctcttatataaaacccactttccccaccaaggggaaagctttctggattctcagaaagcccccttcagggcaggtttggtatatagttggtgcacctgagacaggtggtagtaattttgataattactttaaacttatggttgttattgttttataatgttgttgtatcttctctataccccaaattatccatttttcaagttcctgttgtctctctctctctctctctctctctctctctctctctctctctctctctctctctctctctccctccctccctccctctctctttctctctctttctttttcattcaccttggtttgttagaatggctctgaaatctggtactttgaaaattacaaattacacaaattcttgcctaagaaagactcctcagggctatctgtgttctgttactcaccaaaccttgtccccagccatggctgctgctatcaactactgccaccctcctcccgccactccccacctcagtatttgaaaactcagttgtatcttagcctcttctctttgaaatgtgaacagtttcccatgctcctctttcttcatcctgtgtttgctttaacctggccttctctcctcaaggctcagctagacactgcctgtcagctaccttgagtttcctttgtggcagccttcctaacactgtgacccttcaatacagttcctcacattgtggtgacaccccaaccatacaattattttcatcgatacttcataactgtagttttgttactgttagaattgtaaataaatatctgatatgtttctcctgtgggggctgtgacccccaggttgagaaccactgctgtttcatgggtttggattcacctcccccacttcgtctgtggcacgggattattcaaacgtcttactttttcctttgctgaattttggtaggattttgtttcctagggaatgacacaggtccttgaaggtttcattcacactggcaaattatttatcatctcttctcttcttgtctgtctacttctgcagaggagatcgagtttggtcctctttacagggattctcttcctctctctccctaacctggtataagcccctttgcatgagcagcttgtcgttcaagatgaagccttctgtcttctcagacagcctgcctggcactttgtgatcagctcgatcttcttcgcgttcatatcctttgccgccttgtgtggagtgtggacgggagcatccttacacctgtcttgtgcgactttgacagagtgtgaggttctcaaaggtgcttgatcaccacccctagaattgccagatactaaagatgcaaataaataactcacggttccacgaatgtcttgtagttccacttcctgtcttgtagctccacttccagtcttgtagctctacttcctgtcttgtagctccacttcctgtcttgtttcttagttcccaaccctttcaaggagtttgtcagtctcccccccgccccaacccccacagttgctaactctccgagtttg

SEQ ID NO: 5 - DNA, 1068 bp, synthetic construct
  source 1..1068: mol_type = other DNA
atgtggtccttgctgctctgcgggttgtccatcgcccttccactgtctgtcacagcagatggatgcaaggacatttttatgaaaaatgagatactttcagcaagccagccttttgcttttaattgtacattccctcccataacatctggggaagtcagtgtaacatggtataaaaattctagcaaaatcccagtgtccaaaatcatacagtctagaattcaccaggacgagacttggattttgtttctccccatggaatggggggactcaggagtctaccaatgtgttataaagggtagagacagctgtcatagaatacatgtaaacctaactgtttttgaaaaacattggtgtgacacttccataggtggtttaccaaatttatcagatgagtacaagcaaatattacatcttggaaaagatgatagtctcacatgtcatctgcacttcccgaagagttgtgttttgggtccaataaagtggtataaggactgtaacgagattaaaggggagcggttcactgttttggaaaccaggcttttggtgagcaatgtctcggcagaggacagagggaactacgcgtgtcaagccatactgacacactcagggaagcagtacgaggttttaaatggcatcactgtgagcattacagaaagagctggatatggaggaagtgtccctaaaatcatttatccaaaaaatcattcaattgaagtacagcttggtaccactctgattgtggactgcaatgtaacagacaccaaggataatacaaatctacgatgctggagagtcaataacactttggtggatgattactatgatgaatccaaacgaatcagagaaggggtggaaacccatgtctcttttcgggaacataatttgtacacagtaaacatcaccttcttggaagtgaaaatggaagattatggccttcctttcatgtgccacgctggagtgtccacagcatacattatattacagctcccagctccggattttcgagcttacttgataggagggcttatcgccttggtggctgtggctgtgtctgttgtgtacatatac

SEQ ID NO: 6 - DNA, 2758 bp, synthetic construct
  source 1..2758: mol_type = other DNA
aacagctttaagatcgacatcatgctttggtacagaagcgccttccacactgcccaggctccagatgatgagaagctgtatgatgcctatgtcttataccccaagtacccgagaggaagccagggccatgatgtagacacactggtactgaagatcttgcccgaggtgctggagaaacagtgtggatataagttatttatatttggcagggatgaattccctggacaagctgtggccagcgtcattgatgaaaacattaagctgtgtaggaggctgatggtctttgtggcaccagagtcgtctagcttcggctttctaaagaacttgtcagaagaacaaattgccgtctacaatgccctcatccagcatggcatgaaggtcattctgatcgaactggagaaagtcaaagactacagcaccatgcccgagtccattcagtatatccgacagaagcacggggccatccaatgggatggggacttcacagagcagtcacagtgtgccaagaccaagttctggaagaaagtgagataccatatgccacccaggaggtatccagcatcttccccagtccagctgctgggacacataccctgcaattgcaaggcaggcaaatgcaatgctgccacagggctcataactccctgagagtggttagtgtgtgttggctcacactacatcctctctgaattgtctactcatgtagctggctcttttgtgcttgtgagcgaccttgtccttgcccttgtagctttttgtttatttgtattgtttgctggatgctttcagtcatagctgatccttattactcctgtttgcttcatggctcctgaaaacccatatactccccaagcatggggtggctctaacgttgggcctccgtgtcagtgggatactgaggatgacagatcaggtgtccaggatgcttgatcctggtttgagatgggaggctgggcagtctgaggataccaagtagatagccttagggaagatcagcaaggacagtagtgcaatgtctgtgtttgtccagggtccacaagattctagtcctcaggcagtgtagactgcatttgaatctccttcattttcctattgcatttagttaactaaccattgtgagaacaggacagaggactgggagttgggggtggggggaggtgacagaggatgggagtgtgtgtgtaagaggtgacagttacatgatcaatcctgggaaatcagactcaggaaaccagcattagggtgcagatctgagttttttgtgcagcccttaagcttatatacagcgttcgagccaatcccaagcccctaaaccctcggccaatcatatcttgccacatcatcactatgcctcaatgaaagccctgcctgatgaggtaactcaaaaggaattggggtgggaagcatcatcccctgtcagaacttccgtgaagatggaggacgtagccactaccccacctcagtttctttttgttgtctcaggtttgcaccagagccatcttaaatctagtgacatgtgcagcaaatcatgactcttcaaggagctgcgggagcctgtggtgcctcgctctccatcccactttttccttcacagggttgacttccacgtcccccaaaccatggtattagcacgatgcccctagaagccgcttagatgtcgccaggaccaatggtcttcatttccctctttcccagcccctgtgaatcttactattctaggaaattcaaatgagagacacggtgcattcacggtttctcattttatgactggcttctttcatacaccacggatttctcaagtgctctcgagattcatccattgattagccttttgttttattattatcactactgtctttctcttctttttcttcctctttaaatttctccccctcctcacgagcatccggagaggagcatagactctctgcatgcctacagcgtgatgagctcaccgagggtcccttcaatcacaagcttgtatgttttgcttttcggtttctctttgcaccatgggaattgtaactccttacagaggatttgaaggttcgggtacaaaggcgtcactgactggttccttcttctgtcttcctcaccctgaatgggctttgcttctgggtgattcctggaaattgtcagcagtgatggagaaacctagggacagggctgactaatggggcctggcctttcttctttgaagcccaggacacagtgcctgcctagtgggtcctggacacaccaggtggttccactctgctcagtggtaacattttttagctgagtcatcaacatcgggtcctttagtgaggaggtgcgggcatctctccccagttagatttggactcttacttccctgctcatttccctttgaaagattcaggagactatgtcagaagcagggagccagcaccctgtgtcttctccacgtggctccttgtcacacctgtgaccaccaattttagaaaatgtgtttccttgttctgaccacacttatgcggacattagttgaggctgttgtcagaaaggaaaattaactcatttgaagtaaacattttctggaaatctctgacttctgtcaggccaatggtgctgaaaaatttaaaactgaattcacttttttggccagaaagctttcctcttgccaacatgtcttaattttccttttgcttaaacttattggagatgtattatgaatgctaagatgtgagtgtgttatatcagtacattgacattttaaataaagtatattttaataaaa

SEQ ID NO: 7 - DNA, 80 bp, synthetic construct
  source 1..80: mol_type = other DNA
tacttctaccattgtcctctcttttcctttgtagcctgagatgtggtccttgctgctctgcgggttgtccatcgcccttc

SEQ ID NO: 8 - DNA, 80 bp, synthetic construct
  source 1..80: mol_type = other DNA
cagtacattgacattttaaataaagtatattttaataaaaatcgataccgtcgacctcgactgtgccttctagttgccag

SEQ ID NO: 9 - RNA, 4063 nt, synthetic construct
  source 1..4063: mol_type = other RNA
ggcggacagagaccggaagctgggagatgatggcttctgatgtttgttgcttacgcgacacagcttgtttggaattatgtggcccttgacaactgcgtggcacacacttcaagatacaaaagtgagaagaagaggggggtcaccagaggagactgaagtcagtaagtttctaccaaagctgggtttcttctggccactccaaacaaagctgaaattgtccttcagacctctcctgagatgtggtccttgctgctctgcgggttgtccatcgcccttccactgtctgtcacagcagatggatgcaaggacatttttatgaaaaatgagatactttcagcaagccagccttttgcttttaattgtacattccctcccataacatctggggaagtcagtgtaacatggtataaaaattctagcaaaatcccagtgtccaaaatcatacagtctagaattcaccaggacgagacttggattttgtttctccccatggaatggggggactcaggagtctaccaatgtgttataaagggtagagacagctgtcatagaatacatgtaaacctaactgtttttgaaaaacattggtgtgacacttccataggtggtttaccaaatttatcagatgagtacaagcaaatattacatcttggaaaagatgatagtctcacatgtcatctgcacttcccgaagagttgtgttttgggtccaataaagtggtataaggactgtaacgagattaaaggggagcggttcactgttttggaaaccaggcttttggtgagcaatgtctcggcagaggacagagggaactacgcgtgtcaagccatactgacacactcagggaagcagtacgaggttttaaatggcatcactgtgagcattacagaaagagctggatatggaggaagtgtccctaaaatcatttatccaaaaaatcattcaattgaagtacagcttggtaccactctgattgtggactgcaatgtaacagacaccaaggataatacaaatctacgatgctggagagtcaataacactttggtggatgattactatgatgaatccaaacgaatcagagaaggggtggaaacccatgtctcttttcgggaacataatttgtacacagtaaacatcaccttcttggaagtgaaaatggaagattatggccttcctttcatgtgccacgctggagtgtccacagcatacattatattacagctcccagctccggattttcgagcttacttgataggagggcttatcgccttggtggctgtggctgtgtctgttgtgtacatatacaacagctttaagatcgacatcatgctttggtacagaagcgccttccacactgcccaggctccagatgatgagaagctgtatgatgcctatgtcttataccccaagtacccgagaggaagccagggccatgatgtagacacactggtactgaagatcttgcccgaggtgctggagaaacagtgtggatataagttatttatatttggcagggatgaattccctggacaagctgtggccagcgtcattgatgaaaacattaagctgtgtaggaggctgatggtctttgtggcaccagagtcgtctagcttcggctttctaaagaacttgtcagaagaacaaattgccgtctacaatgccctcatccagcatggcatgaaggtcattctgatcgaactggagaaagtcaaagactacagcaccatgcccgagtccattcagtatatccgacagaagcacggggccatccaatgggatggggacttcacagagcagtcacagtgtgccaagaccaagttctggaagaaagtgagataccatatgccacccaggaggtatccagcatcttccccagtccagctgctgggacacataccctgcaattgcaaggcaggcaaatgcaatgctgccacagggctcataactccctgagagtggttagtgtgtgttggctcacactacatcctctctgaattgtctactcatgtagctggctcttttgtgcttgtgagcgaccttgtccttgcccttgtagctttttgtttatttgtattgtttgctggatgctttcagtcatagctgatccttattactcctgtttgcttcatggctcctgaaaacccatatactccccaagcatggggtggctctaacgttgggcctccgtgtcagtgggatactgaggatgacagatcaggtgtccaggatgcttgatcctggtttgagatgggaggctgggcagtctgaggataccaagtagatagccttagggaagatcagcaaggacagtagtgcaatgtctgtgtttgtccagggtccacaagattctagtcctcaggcagtgtagactgcatttgaatctccttcattttcctattgcatttagttaactaaccattgtgagaacaggacagaggactgggagttgggggtggggggaggtgacagaggatgggagtgtgtgtgtaagaggtgacagttacatgatcaatcctgggaaatcagactcaggaaaccagcattagggtgcagatctgagttttttgtgcagcccttaagcttatatacagcgttcgagccaatcccaagcccctaaaccctcggccaatcatatcttgccacatcatcactatgcctcaatgaaagccctgcctgatgaggtaactcaaaaggaattggggtgggaagcatcatcccctgtcagaacttccgtgaagatggaggacgtagccactaccccacctcagtttctttttgttgtctcaggtttgcaccagagccatcttaaatctagtgacatgtgcagcaaatcatgactcttcaaggagctgcgggagcctgtggtgcctcgctctccatcccactttttccttcacagggttgacttccacgtcccccaaaccatggtattagcacgatgcccctagaagccgcttagatgtcgccaggaccaatggtcttcatttccctctttcccagcccctgtgaatcttactattctaggaaattcaaatgagagacacggtgcattcacggtttctcattttatgactggcttctttcatacaccacggatttctcaagtgctctcgagattcatccattgattagccttttgttttattattatcactactgtctttctcttctttttcttcctctttaaatttctccccctcctcacgagcatccggagaggagcatagactctctgcatgcctacagcgtgatgagctcaccgagggtcccttcaatcacaagcttgtatgttttgcttttcggtttctctttgcaccatgggaattgtaactccttacagaggatttgaaggttcgggtacaaaggcgtcactgactggttccttcttctgtcttcctcaccctgaatgggctttgcttctgggtgattcctggaaattgtcagcagtgatggagaaacctagggacagggctgactaatggggcctggcctttcttctttgaagcccaggacacagtgcctgcctagtgggtcctggacacaccaggtggttccactctgctcagtggtaacattttttagctgagtcatcaacatcgggtcctttagtgaggaggtgcgggcatctctccccagttagatttggactcttacttccctgctcatttccctttgaaagattcaggagactatgtcagaagcagggagccagcaccctgtgtcttctccacgtggctccttgtcacacctgtgaccaccaattttagaaaatgtgtttccttgttctgaccacacttatgcggacattagttgaggctgttgtcagaaaggaaaattaactcatttgaagtaaacattttctggaaatctctgacttctgtcaggccaatggtgctgaaaaatttaaaactgaattcacttttttggccagaaagctttcctcttgccaacatgtcttaattttccttttgcttaaacttattggagatgtattatgaatgctaagatgtgagtgtgttatatcagtacattgacattttaaataaagtatattttaataaaa

SEQ ID NO: 10 - protein, 571 aa, synthetic construct
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNSFKIDIMLWYRSAFHTAQAPDDEKLYDAYVLYPKYPRGSQGHDVDTLVLKILPEVLEKQCGYKLFIFGRDEFPGQAVASVIDENIKLCRRLMVFVAPESSSFGFLKNLSEEQIAVYNALIQHGMKVILIELEKVKDYSTMPESIQYIRQKHGAIQWDGDFTEQSQCAKTKFWKKVRYHMPPRRYPASSPVQLLGHIPCNCKAGKCNAATGLITP

SEQ ID NO: 11 - DNA, 80 bp, synthetic construct
  source 1..80: mol_type = other DNA
agggggaggattgggaagacaatagcaggcatgctggggagatatcgaattccgaagttcctattctctagaaagtatag

SEQ ID NO: 12 - DNA, 80 bp, synthetic construct
  source 1..80: mol_type = other DNA
ataggaacttcatcagtcaggtacataattaggtggatccggggttacatctttgctcttctgtggggtgtttttcctgc

SEQ ID NO: 13 - DNA, 25 bp, synthetic construct
  source 1..25: mol_type = other DNA
ctgaggctctgtggtgctggttatc

SEQ ID NO: 14 - DNA, 27 bp, synthetic construct
  source 1..27: mol_type = other DNA
tgtccttgcatccatctgctgtgacag

SEQ ID NO: 15 - DNA, 20 bp, synthetic construct
  source 1..20: mol_type = other DNA
gctcgactagagcttgcgga

SEQ ID NO: 16 - DNA, 25 bp, synthetic construct
  source 1..25: mol_type = other DNA
gtcaagcaagatgcggcttagaagg

SEQ ID NO: 17 - DNA, 26 bp, synthetic construct
  source 1..26: mol_type = other DNA
ctaaccatcttctttctgcagatgac

SEQ ID NO: 18 - DNA, 25 bp, synthetic construct
  source 1..25: mol_type = other DNA
gctagttttccagacggctgtccat

SEQ ID NO: 19 - DNA, 25 bp, synthetic construct
  source 1..25: mol_type = other DNA
ctggttctcctaatccctttgtttg

SEQ ID NO: 20 - DNA, 30 bp, synthetic construct
  source 1..30: mol_type = other DNA
ctttcagtatttacattctcttcaatacac

SEQ ID NO: 21 - DNA, 21 bp, synthetic construct
  source 1..21: mol_type = other DNA
ggatcggccattgaacaagat

SEQ ID NO: 22 - DNA, 22 bp, synthetic construct
  source 1..22: mol_type = other DNA
cagaagaactcgtcaagaaggc

SEQ ID NO: 23 - DNA, 25 bp, synthetic construct
  source 1..25: mol_type = other DNA
ggcgtttactggcttctgaaggtct

SEQ ID NO: 24 - DNA, 25 bp, synthetic construct
  source 1..25: mol_type = other DNA
aaagagtgactagcaaggttggggc

SEQ ID NO: 25 - DNA, 25 bp, synthetic construct
  source 1..25: mol_type = other DNA
agctttcctcttgccaacatgtctt

SEQ ID NO: 26 - DNA, 25 bp, synthetic construct
  source 1..25: mol_type = other DNA
aaagagtgactagcaaggttggggc

SEQ ID NO: 27 - DNA, 25 bp, synthetic construct
  source 1..25: mol_type = other DNA
gacaagcgttagtaggcacatatac

SEQ ID NO: 28 - DNA, 24 bp, synthetic construct
  source 1..24: mol_type = other DNA
gctccaatttcccacaacattagt

SEQ ID NO: 29 - protein, 160 aa, Mus musculus
MNKEKELRAASPSLRHVQDLSSRVWILQNNILTAVPRKEQTVPVTITLLPCQYLDTLETNRGDPTYMGVQRPMSCLFCTKDGEQPVLQLGEGNIMEMYNKKEPVKASLFYHKKSGTTSTFESAAFPGWFIAVCSKGSCPLILTQELGEIFITDFEMIVVH

SEQ ID NO: 30 - protein, 158 aa, Homo sapiens
MEKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 31 - DNA, 3247 bp, synthetic construct
  source 1..3247: mol_type = other DNA
atggggtattatactagtttctttctggggcctcaaaagttggaaactgccattgtattccaggcgtgggtcaagggtcaaatgtactgatggatgcatgtcttctcaatggttcatgttgttactgagtctgcactgaggaagtatctacggtccaccagagttgtcagtacagtggtagctttctttcttcatgtgacaggacctctgctggcaatgcttagatttacatctcatggtcttgaatattaattccccatctcaacactctccagattgtatggtctagctaatatgttagctcaatatctgccaagtgcattaggactgtggaaagcaaacagtacagaaattggtatgggtgaaagatttgtcttcatactgtcaaaacattcttattattaattgccctgctggaggaatatagttatcggtgtcttgttgtggtgaattaaataaaaattaccaccatagactcatagggagtggcactattaggagatgtagccttgttggagtaggtgtgtccatattgaaggaaatatatcactggggacaggctttgagctctcaggccaggcccagtgtctcactctcttcctgctctctctgccaatctagatgtagaactccagcaccaggtctgcctggatgctgccttgttttccaccataatgacaatcaaataaacctctgaattgtaagctagccccaattaaatattttcctttgaagagttgttttggtcatggcgtcttttcatagcaatagaaaccctaactaagacatttgtacaaccttggaaatttctcaggctctacagcagtgaacaaagtttcccaaattagatctgtataccacccttgggctcaaagctaccaaattcttaagagttattttcagtagatgaaatctgttcctagctatccaagataagctgtctctgattctccagcttggcttcattctgtctttctctttctcctttggcttggtgccaacaaagcccatttgtatagttttcagagattagaggaaaaatggcttcagggctctgaacctgtttcctcccagccccaagcatgcccagagccctgaccacttgcttttggtgttgatttgattgttacagagcatgggccttatattttaaacacagaaggcatagcaataaaattttttgagatagaatctgtctacattgccctgaatctcctggaactcaccatgtagaccaggctgagctcaaactcacagagatccgcttgcctctgccttcagagtgttgagatttaaggcatgcaccaccacgccaggctggtatagcaatttttaatgttcttttctttcagatcttgattctgtaggttggagtagagcctgggcttttgtatttctgaccagatgtcaaatgatgccaatacaacatgcataatctacactttagaatcaattgttagcacagttataaatgtttaaagttttactttgaatatatattatttgaatgcaaatgtcttccttttgaatattgaactattggcttgttggggttttctttagagcagggcacttgtttaggagaagactacagggaattcagtagacactagttacttttataggtaactgtggctgggacccactaggggccctctaagaagcctgagaattgcctgtcagaagactaggaggccgggaccttatccaccagtcccttaactcattggtagtatttgtccctcagatggaacccactaaggtatgaaagtgacatgttttcagatctttcagaggcacactgtaactcctatacaggaaaaggcatcaaaatcattgcctcagtttcctttcagaaaaatctaacgatgggcactctgacaactgatagaccatctgacaactaggcctaagcatgacaaacatggcaccatcaaatgctgaccttcctgctcccctctcttgcaaaaccatcggattcccttatttggccacttccttgttgtgcctgactccttcctgggggtaactacggagatccagctatcaaattttcctattttgctacattgactaacaagtccaaccagaacctacctactcattctagcccactctaccatgatcttcctcctttttccctttaaaaataattcttacagatccatttactgctgcttctgcccatttcagatacagccaccttgtgtctctgtcttgcttaataaaattgctgtttgggtgtgatctgtgcctaatagagtgtcgaaagggagcaccccaaatgtgagtggttcatcagacttcctcttcctataacaagataatgacttaaatctgctgaaatgtggacaggcagtccaggagatgtctttgctttggtcagaacttgcggtggagaagcaacgtgaatattggctgggagaaggtggaatatccactggagtcagcctgaggtgacttgtatcagtttaaaaataatataggacttagagaatcgtcttcttatacacacttgcacttcctgtaggttcctactaatagaagcaatattctagtacttcatcttacgtcacctagtccagtcatccatcctggggaaatcccatgaagtttctttcctccatcaggttaaaatatgtgacctatttcctgattactgctctcacaggtttagcaagccctccctttccaatgtttcctaagtcacaagcatactacaattgccatttattccaacagatgcttaacaagccctccagcacatggaaccagcaattatctagggtgtccagcagtcctaggtgcactgaggcataacctcctcttttcgtgttgtggataaaaggcttggaccttagtagagcttgtcagagaagctgagcacagtagtgggtgtagttctgtagtgtgcagacacattcctattcaatcaggttagaaacgcagcctgtggctttcagccactgcccagaatctcttgctgcttggctctgaccttggtgctctctgtccccttgtacctcaggcctacatgggaactagagtagctgcttctcttggacaataagatgaatctaaggatacaatgggaccttgtgacgcttggtttagagaatctgcctggggtcacagttaaggcgttcaccagctcatctgacctgggtcaaactgtgacttaggactaactctgtgtctttcctagggtcaatctgcagattggcagctcagaaacaacatcaccata

SEQ ID NO: 32 - DNA, 3602 bp, synthetic construct
  source 1..3602: mol_type = other DNA
ctctaccttcagaggtatgggattacaaatgggacgcacgctggttaaggtgttttgttttgttttgttttgttttgttttgttttgttttgttttcaacttgaggcaagcagaggtcatctgagaagaggggaacttcagttgtgtagaattttcttgattaatgactaatgtgggagagtctaatctataatgggcagatcctaccctgtgctaataattcagtggtataagaaagtaaaccttaaaatgagcaagccagtaagcagcactcctccaaaggtctctgcttcaatttttgcctctgggttttcatctgcttgaattcttgccttggcttctgtagcatctgaatgataataccctcatatactgacatgcttagtccctagttgatgaactctttggaaggattgcaaagtggggccttgttgtaggatatgtgttgctttgagatttcaaatgtccataccagtcccagaattgttctttctgccttctgcttgaggatcaagatgtaaagctcttagttcttgctccagtgctatgtatgcctgcttcacaccatgataacaatgtaataaccctctaaaactgtaagcaagaacccagttaaatgctggtttttaaaaatgtcaccttagacacaatgtctcttcatagtaataaaacggtaaccagggcagtatccctcaaaagagggctttgaccagaatatgtaaaccaaatgaatcttttcctctccaaattgcttttggctatctttataaaagcaataggtagcaaattaatacaactgctatgcctgcacagcccttccataggtactaaggatttaaactctcgttttcatacttgagcagcaactatactatcctttttttccatctcctcagccccaagtctcttagcttttagaactaacaagatttccaaagaagtcttaaacttcttggtagtctcttccaaaaccagattcttcaagtcacaaatatggaatgactaagacaaacaggactccattaataaaaattgtattctatatttcatagtctatattgcacaatgcagacacataactgacttcaaatctccttcctgagatgtgtttctgtgggcatcttttgcctttttcttttaactcatcaaccataaaatgacttcactctcttatatggagaggctcttcattttcaaaggattatcttttcctatatctttttaaaattaaagtttcttttaaaattcctgtccaaaggaaagacaggggtaaaaaactggaggagagactgaaggaagggtcaactggggactgctccacttggaaattcatcctgtctacagacaccaaacatgacacagttgctgttgctgttgccaagaggcacttgatgacaggaacctggcacagttgatccctgggaggctctgctagcacctgaccaacgcagatgcagatacttggagccaaccatcagattgagctttgggacccaggtgtgggagctgatggaaggacggaaggagcagaaagggattgcaaccccataagaagaacaatgtcttttggccagactacccactgctcccagggactagaccaccaaacaaagagggatccatggctccagatacatgcatatagcaaagaatggccttgtttgacattaatgggaggggaggcccttggtcttgtggaggtttgatgccccagtgaaggaggatgctggaatgttggggcaggagtgagtgggtgggtggaggaacaccctcataaaggcaaaagggagggtccattgggggggtattttgactacctcactccaaatttgtatctctttgatttctttttgctgcctaatttctctggctagaatttgagtactatactgaatagttacagagagggtgagcagccttgtcatgtccctgattttattgggattgtctcaagtttctctccatttagttcgatgttggctactggtttgctctatattacttcttttttaaaattatttttattttttattaggtattttcttcatttacatttccagtgctatcccaaaagtcccccataccctcccaccccactcccctacccatccactcccacttcttggccctggcgttcccctacactgaggcatataaagtttgcaagaccaatattacttctattatgcttagaaatggactttaaattcttgatctctcccatacttttaacatgaaggggtattatacattgtcaaaggcttcttcagcatctaatgagatggtcatggagtctttttttagtttatataatggattaaattgatggatttctgtatattgaaccatccctgcattcctgggaagaagcctagttgatcatggtaaatgatcattttgatgtgttcttggattcagtttgcaagaattttgttgagtatttttccatcagtattcataagggaagttggtctgaagttctctttctttattgggtctttgtgtggtttaggtataagtgtaactgtggcttcacagaacatattgggtagtgttccttctgtttctattttgtgggatagtttgaaaaagtattagtcttaggtcttctttgaagatctgatagaattctgaactaaaaccatctggtcctgggttttttcttgatagagaaaaattttaatgactgcttctatttttttaggggttagggaactgttttaatggtttatctgatactgattaaactttggaacctaatatctgtctagaaaatcaactatttcacccagattttccagttttattgagtacaggcttttgtagtaggatctgatgatttttttaaatttcttcagtttctgttgttatgtttcccttttcatttctgattttgttaattaggaagccaactctaagatggcgccggtttccggtgtgccgttgtcataaacaacaccactgcgcaggcataagcccgagtctggtgcccacccctcccaagcgggcacatgctaattaggtgacagcagttcaacctatcccaggaggacacgaagcctttccctgtgctggggtgtatataagcagatctccctgggttcccggggtccccgtagcagcatcaaggcattcctgcaataaagactgttgagaataatcctaccgtgttgtgtcttccgtgctggccaaggtgggcgcaatagtctctgtgctctctggttagtctgggtaagggtttatttatcttgttcattttctcaaagaaccagctcctggttttgttgattctttgtgtagttatcctcatttctacttggttgatttcaaccctgagtttgattatttcctgccatctacttctcttgggtgtacttgtttatttttgttctagagctttcagatatgctgtaagctatctatgtattctctctccagtttctttatggaggcactcagagctatgagttttcctcttagtactgctttcattgtgtcctataagtttgggtatattgtgccttaatttttggtaaattataaaaagtctttaatttctttatttacttctttcctaacaaagttatcattgagttaatgat

SEQ ID NO: 33 - DNA, 2173 bp, synthetic construct
  source 1..2173: mol_type = other DNA
atggaaaaaggtaaagatcctcgtggaagggcgaaaaattgacaagggggtgatattctgtgctctcattcttactaatttacattttgactttctcaacagcattgaaaattgacacacctcagcaggggagcattcaggatatcaatcatcgggtgtgggttcttcaggaccagacgctcatagcagtcccgaggaaggaccgtatgtctccaggtgagtagccacggtctgtgaaaggcagctcctttggccagtgctgttgtgtgtttggtgctcagatgttattccacagtgggcttgttaaccgggcatatctacagagaggagcatctcacagacaataatgaatagagggtgagcataaggttgttctgggactatggtcatgaagtcagagaagagagagaaggcttaagggagactgaagggtcagcctatgaaaaacagtggaagacgtattctgagaaacttcaaggagtaagagtaggacaaaagagggtaatctatgggaggaccagtttttgaatccatgaaagactgacttttctaatggaaagtgttccccaaacataggatgggtgtcccggttaggtggtgggtggtgaggtgaccattctatgagtgcactgtgagtgtcagttagcagattcccttaaaactctggctcagaggtaccaccctggaaatggcagctgaacaccacttttcacatcgtgttcccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggtgagtgtggaagacaggtcctgccatttatttgatggaaactcagatttcaggatcttttaaaagacaatgtacttattatgctcatgcatttttatttttggtttccttggggttggtatatgttatgaaagactaagaaaaattgcagggacattttgaaacaaacctactaaaatgacttattattttaatttacaaaaatattaataatttggcaaggatgatgtctaacttcaatagaacacacacacagagtaaaatcagcaaggacattttagcagttaattatcacaaagtagacgaacttattaaaacatctttttggcaaaaaatacattgcaactttaaatgaacaaacataattctctgtaataaacacttcaataatcgcaattttccattattaaaaaaagataattatagcaggcctctgtgtactacaggaactctcttgacccttttcagtaaacaagcataagcagacactcaacttgatgacaagatataaaacagtgtttttcagccaggggtgatattgctccagggacatttgtcaatgtctggagatgttttggttgtgacaacctgggatgggggtgtgatacttgcatttatcgggtagatgacagggatgctactaaacatcctatattattatacaggacagaccctgaaaacaaagaatcatcttgcctaaaatgtcaatagtgctgaaggtgcagaaacctgacagaaaataactgttttaatttgaaatttacagtaactgttacataaatattctttaagtttctgtttctgaacaacaaataatggcccaatcaatgctttgtattctcaaaaactttcatgtcaacacatttttcagagcctcacattaaaaaccattaaaatttgtagcctgagtttcaaccataatcttaaaagaatttaatttatatgattttcaatcccataatatgaaacagaggtctccaagctgcttcaatgagtagggaatgctatccttggacgtggaatatcagtgtgtaaagaatgtgaaacattcaaacagttatgtttcttgctggtgtctccttcgcacccttcaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttttaa

SEQ ID NO: 34 - DNA, 80 bp, synthetic construct
  source 1..80: mol_type = other DNA
agccaacactactgactttgggttaactatgctgttttaaggtcagtcttgtaatgggtgctgcttcccaagcagagaac

SEQ ID NO: 35 - RNA, 877 nt, synthetic construct
  source 1..877: mol_type = other RNA
taacctcctcttttcgtgttgtggataaaaggcttggaccttagtagagcttgtcagagaagctgagcacagtagtgggtgtagttctgtagtgtgcagacacattcctattcaatcagggtcaatctgcagattggcagctcagaaacaacatcaccataatggaaaaagcattgaaaattgacacacctcagcaggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttttaaggtttttagacacattgctctgtggcactctctcaagatttcttggattctaacaagaagcaatcaaagacacccctaacaaaatggaagactgaaaagaaagctgagccctccctgggctgtttttccttggtggtgaatcagatgcagaacatcttaccatgttttcatccaaagcatttactgttggtttttacaaggagtgaattttttaaaataaaatcatttatctcatttgc

SEQ ID NO: 36 - DNA, 80 bp, synthetic construct
  source 1..80: mol_type = other DNA
agctggtgtagctagccagcttgctcatagagaactctgtgaagttcctattctctagaaagtataggaacttcaggtct

SEQ ID NO: 37 - DNA, 80 bp, synthetic construct
  source 1..80: mol_type = other DNA
cccttcgaagttcctattctctagaaagtataggaacttcctctaccttcagaggtatgggattacaaatgggacgcacg

SEQ ID NO: 38 - DNA, 1887 bp, synthetic construct
  source 1..1887: mol_type = other DNA
tctgtaggttggagtagagcctgggcttttgtatttctgaccagatgtcaaatgatgccaatacaacatgcataatctacactttagaatcaattgttagcacagttataaatgtttaaagttttactttgaatatatattatttgaatgcaaatgtcttccttttgaatattgaactattggcttgttggggttttctttagagcagggcacttgtttaggagaagactacagggaattcagtagacactagttacttttataggtaactgtggctgggacccactaggggccctctaagaagcctgagaattgcctgtcagaagactaggaggccgggaccttatccaccagtcccttaactcattggtagtatttgtccctcagatggaacccactaaggtatgaaagtgacatgttttcagatctttcagaggcacactgtaactcctatacaggaaaaggcatcaaaatcattgcctcagtttcctttcagaaaaatctaacgatgggcactctgacaactgatagaccatctgacaactaggcctaagcatgacaaacatggcaccatcaaatgctgaccttcctgctcccctctcttgcaaaaccatcggattcccttatttggccacttccttgttgtgcctgactccttcctgggggtaactacggagatccagctatcaaattttcctattttgctacattgactaacaagtccaaccagaacctacctactcattctagcccactctaccatgatcttcctcctttttccctttaaaaataattcttacagatccatttactgctgcttctgcccatttcagatacagccaccttgtgtctctgtcttgcttaataaaattgctgtttgggtgtgatctgtgcctaatagagtgtcgaaagggagcaccccaaatgtgagtggttcatcagacttcctcttcctataacaagataatgacttaaatctgctgaaatgtggacaggcagtccaggagatgtctttgctttggtcagaacttgcggtggagaagcaacgtgaatattggctgggagaaggtggaatatccactggagtcagcctgaggtgacttgtatcagtttaaaaataatataggacttagagaatcgtcttcttatacacacttgcacttcctgtaggttcctactaatagaagcaatattctagtacttcatcttacgtcacctagtccagtcatccatcctggggaaatcccatgaagtttctttcctccatcaggttaaaatatgtgacctatttcctgattactgctctcacaggtttagcaagccctccctttccaatgtttcctaagtcacaagcatactacaattgccatttattccaacagatgcttaacaagccctccagcacatggaaccagcaattatctagggtgtccagcagtcctaggtgcactgaggcataacctcctcttttcgtgttgtggataaaaggcttggaccttagtagagcttgtcagagaagctgagcacagtagtgggtgtagttctgtagtgtgcagacacattcctattcaatcaggttagaaacgcagcctgtggctttcagccactgcccagaatctcttgctgcttggctctgaccttggtgctctctgtccccttgtacctcaggcctacatgggaactagagtagctgcttctcttggacaataagatgaatctaaggatacaatgggaccttgtgacgcttggtttagagaatctgcctggggtcacagttaaggcgttcaccagctcatctgacctgggtcaaactgtgacttaggactaactctgtgtctttcctagggtcaatctgcagattggcagctcagaaacaacatcaccata

SEQ ID NO: 39 - DNA, 980 bp, synthetic construct
  source 1..980: mol_type = other DNA
ggtcagtcttgtaatgggtgctgcttcccaagcagagaacctcacctttgcttccaccattctagttctccaagccattcttttttttacatgaatactgttatcctgcttcatccttgagaggaagtggctttgttcatgttaacctaaaagttagactcatctgggaagcaaaaatttcatttgagaaaatatctccattatattggcttgtcagcaagtctaaatgatctaaatgctgctattttcttgattagtgggtgatacaggagggccaagaccattgtggctggtgctacacctgggtaggtggttccagggggtataagacggcaagctgagtaaaccacacagataaatcagtattcctctgtggtctctgcttccaatctagcctccagattcctgtctagagttcttgcactgacttccatcagtgattcccaactgttaactggtacttggaagcttaaattgaaacaaaacatttccttgctaagttgtttttggtaaatgtttcatcccagaaacaaagaagcaaacagacatacccaattttcaaagatagaaaacagattagaaggtgatgcgtagcaaagcttaatcctagaataaaagaaagaatctgttactggccttttgactcaaggtgaattggtgccctccctccaacatatttatggattgatctaaatgctggtgtaggctatgggggaaatgatagtggtatcctagacttctttgtcatggtcatgtagttaaatcatgacatcatcacaaaactaatgaagagacctctagctatatacttcctggtaatatgtaagctaatatataaatggtgatcatctgtgttcacactttaggtttttagacacattgctctgtggcactctctcaagatttcttggattctaacaagaagcaatcaaagacacccctaacaaaatggaagactgaaaagaaagctgagccctccc

SEQ ID NO: 40 - DNA, 23 bp, synthetic construct
  source 1..23: mol_type = other DNA
tcctgaacatgtctaagcgaagg

SEQ ID NO: 41 - DNA, 23 bp, synthetic construct
  source 1..23: mol_type = other DNA
aggatcttagtagtcgtgtgtgg

SEQ ID NO: 42 - DNA, 23 bp, synthetic construct
  source 1..23: mol_type = other DNA
accttcgcttagacatgttcagg

SEQ ID NO: 43 - DNA, 23 bp, synthetic construct
  source 1..23: mol_type = other DNA
ctttccttgggactgcagtgagg

SEQ ID NO: 44 - DNA, 23 bp, synthetic construct
  source 1..23: mol_type = other DNA
accataatgaataaggagaaagg

SEQ ID NO: 45 - DNA, 23 bp, synthetic construct
  source 1..23: mol_type = other DNA
cactgacttcgagatgattgtgg

SEQ ID NO: 46 - DNA, 23 bp, synthetic construct
  source 1..23: mol_type = other DNA
ggctgcagactcaaatgtagagg

SEQ ID NO: 47 - DNA, 23 bp, synthetic construct
  source 1..23: mol_type = other DNA
gttcttgggtcagaatgagtggg

SEQ ID NO: 48 - DNA, 23 bp, synthetic construct
  source 1..23: mol_type = other DNA
cattaaggtcagtcttgtaatgg

SEQ ID NO: 49 - DNA, 23 bp, synthetic construct
  source 1..23: mol_type = other DNA
gagatgattgtggtacattaagg

SEQ ID NO: 50 - DNA, 20 bp, synthetic construct
  source 1..20: mol_type = other DNA
aggatcttagtagtcgtgtg

SEQ ID NO: 51 - DNA, 24 bp, synthetic construct
  source 1..24: mol_type = other DNA
taggaggatcttagtagtcgtgtg

SEQ ID NO: 52 - DNA, 20 bp, synthetic construct
  source 1..20: mol_type = other DNA
cacacgactactaagatcct

SEQ ID NO: 53 - DNA, 24 bp, synthetic construct
  source 1..24: mol_type = other DNA
aaaccacacgactactaagatcct

SEQ ID NO: 54 - DNA, 20 bp, synthetic construct
  source 1..20: mol_type = other DNA
gttcttgggtcagaatgagt

SEQ ID NO: 55 - DNA, 24 bp, synthetic construct
  source 1..24: mol_type = other DNA
tagggttcttgggtcagaatgagt

SEQ ID NO: 56 - DNA, 20 bp, synthetic construct
  source 1..20: mol_type = other DNA
actcattctgacccaagaac

SEQ ID NO: 57 - DNA, 24 bp, synthetic construct
  source 1..24: mol_type = other DNA
aaacactcattctgacccaagaac

SEQ ID NO: 58 - DNA, 132 bp, synthetic construct
  source 1..132: mol_type = other DNA
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 59 - DNA, 25 bp, synthetic construct
  source 1..25: mol_type = other DNA
ccctgaccacttgcttttggtgttg

SEQ ID NO: 60 - DNA, 25 bp, synthetic construct
  source 1..25: mol_type = other DNA
tgaaaagggtcaagagagttcctgt

SEQ ID NO: 61 - DNA, 25 bp, synthetic construct
  source 1..25: mol_type = other DNA
atctatgggaggaccagtttttgaa

SEQ ID NO: 62 - DNA, 27 bp, synthetic construct
  source 1..27: mol_type = other DNA
tcattgtattcaaaaatgcaaatgaga

SEQ ID NO: 63 - DNA, 25 bp, synthetic construct
  source 1..25: mol_type = other DNA
ctttcacagaccgtggctactcacc

SEQ ID NO: 64 - DNA, 26 bp, synthetic construct
  source 1..26: mol_type = other DNA
tccttggacgtggaatatcagtgtgt

SEQ ID NO: 65 - DNA, 26 bp, synthetic construct
  source 1..26: mol_type = other DNA
actctcgttttcatacttgagcagca

SEQ ID NO: 66 - DNA, 28 bp, synthetic construct
  source 1..28: mol_type = other DNA
gaagagcctctccatataagagagtgaa

SEQ ID NO: 67 - DNA, 25 bp, synthetic construct
  source 1..25: mol_type = other DNA
gcttaagggagactgaagggtcagc

SEQ ID NO: 68 - DNA, 25 bp, synthetic construct
  source 1..25: mol_type = other DNA
ccgactttagcacacatcaggcaga

SEQ ID NO: 69 - DNA, 228 bp, synthetic construct
  source 1..228: mol_type = other DNA
atcgataccgtcgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 70 - DNA, 25 bp, synthetic construct
  source 1..25: mol_type = other DNA
ggactgtaacgagattaaaggggag

SEQ ID NO: 71 - DNA, 25 bp, synthetic construct
  source 1..25: mol_type = other DNA
tgacaagttctttagaaagccgaag

SEQ ID NO: 72 - DNA, 25 bp, synthetic construct
  source 1..25: mol_type = other DNA
gtgaacaccaaggaagttgagtatg

SEQ ID NO: 73 - DNA, 25 bp, synthetic construct
  source 1..25: mol_type = other DNA
ttatatccacactgtttctccagca

SEQ ID NO: 74 - DNA, 22 bp, synthetic construct
  source 1..22: mol_type = other DNA
tcaccatcttccaggagcgaga

SEQ ID NO: 75 - DNA, 21 bp, synthetic construct
  source 1..21: mol_type = other DNA
gaaggccatgccagtgagctt

SEQ ID NO: 76 - DNA, 22 bp, synthetic construct
  source 1..22: mol_type = other DNA
gacccttgagaaagacagaggg

SEQ ID NO: 77 - DNA, 22 bp, synthetic construct
  source 1..22: mol_type = other DNA
agtcagtagtgttggctttccc

SEQ ID NO: 78 - DNA, 22 bp, synthetic construct
  source 1..22: mol_type = other DNA
agcatcaccttcgcttagacat

SEQ ID NO: 79 - DNA, 22 bp, synthetic construct
  source 1..22: mol_type = other DNA
ttcttgggtcagaatgagtggg

SEQ ID NO: 80 - protein, 561 aa, Rattus norvegicus
MGMPPLLFCWVSFVLPLFVAAGNCTDVYMHHEMISEGQPFPFNCTYPPVTNGAVNLTWHRTPSKSPISINRHVRIHQDQSWILFLPLALEDSGIYQCVIKDAHSCYRIAINLTVFRKHWCDSSNEESSINSSDEYQQWLPIGKSGSLTCHLYFPESCVLDSIKWYKGCEEIKVSKKFCPTGTKLLVNNIDVEDSGSYACSARLTHLGRIFTVRNYIAVNTKEVGSGGRIPNITYPKNNSIEVQLGSTLIVDCNITDTKENTNLRCWRVNNTLVDDYYNDSKRIQEGIETNLSLRNHILYTVNITFLEVKMEDYGHPFTCHAGVSAAYIILKRPAPDFRAYLIGGLMAFLLLAVSILYIYNTFKVDIVLWYRSTFHTAQAPDDEKLYDAYVLYPKYPRESQGHDVDTLVLKILPEVLEKQCGYKLFIFGRDEFPGQAVASVIDENIKLCRRLMVLVAPETSSFSFLKNLTEEQIAVYNALVQDGMKVILIELERVKDYSTMPESIQYIRQKHGAIQWDGDFTEQAQCAKTKFWKKVRYHMPPRRYPASPPVQLLGHTPRIPG

SEQ ID NO: 81 - protein, 160 aa, Rattus norvegicus
MNKDKELSAATPWFRHIQDLSSRVWVLQDNILTAVPRKEQTVPVTITLLPCQYLDTLEKNKGDPMYLGVKKPQSCLSCTKNGEQPVLQLREGNILDMYHQKERVKASLFYHKKSGTTSTFESAAFPGWFIAVCSKGSCPLVLTQELGKTFITDFEMTVVH